SEQUENCE LISTING (9 sequences)

SEQ ID NO: 1 - DNA, 4089 bp, Homo sapiens
atgtctgcggagagcggccctgggacgagattgagaaatctgccagtaatgggggatggactagaaacttcccaaatgtctacaacacaggcccaggcccaaccccagccagccaacgcagccagcaccaaccccccgcccccagagacctccaaccctaacaagcccaagaggcagaccaaccaactgcaatacctgctcagagtggtgctcaagacactatggaaacaccagtttgcatggcctttccagcagcctgtggatgccgtcaagctgaacctccctgattactataagatcattaaaacgcctatggatatgggaacaataaagaagcgcttggaaaacaactattactggaatgctcaggaatgtatccaggacttcaacactatgtttacaaattgttacatctacaacaagcctggagatgacatagtcttaatggcagaagctctggaaaagctcttcttgcaaaaaataaatgagctacccacagaagaaaccgagatcatgatagtccaggcaaaaggaagaggacgtgggaggaaagaaacagggacagcaaaacctggcgtttccacggtaccaaacacaactcaagcatcgactcctccgcagacccagacccctcagccgaatcctcctcctgtgcaggccacgcctcaccccttccctgccgtcaccccggacctcatcgtccagacccctgtcatgacagtggtgcctccccagccactgcagacgcccccgccagtgcccccccagccacaacccccacccgctccagctccccagcccgtacagagccacccacccatcatcgcggccaccccacagcctgtgaagacaaagaagggagtgaagaggaaagcagacaccaccacccccaccaccattgaccccattcacgagccaccctcgctgcccccggagcccaagaccaccaagctgggccagcggcgggagagcagccggcctgtgaaacctccaaagaaggacgtgcccgactctcagcagcacccagcaccagagaagagcagcaaggtctcggagcagctcaagtgctgcagcggcatcctcaaggagatgtttgccaagaagcacgccgcctacgcctggcccttctacaagcctgtggacgtggaggcactgggcctacacgactactgtgacatcatcaagcaccccatggacatgagcacaatcaagtctaaactggaggcccgtgagtaccgtgatgctcaggagtttggtgctgacgtccgattgatgttctccaactgctataagtacaaccctcctgaccatgaggtggtggccatggcccgcaagctccaggatgtgttcgaaatgcgctttgccaagatgccggacgagcctgaggagccagtggtggccgtgtcctccccggcagtgccccctcccaccaaggttgtggccccgccctcatccagcgacagcagcagcgatagctcctcggacagtgacagttcgactgatgactctgaggaggagcgagcccagcggctggctgagctccaggagcagctcaaagccgtgcacgagcagcttgcagccctctctcagccccagcagaacaaaccaaagaaaaaggagaaagacaagaaggaaaagaaaaaagaaaagcacaaaaggaaagaggaagtggaagagaataaaaaaagcaaagccaaggaacctcctcctaaaaagacgaagaaaaataatagcagcaacagcaatgtgagcaagaaggagccagcgcccatgaagagcaagccccctcccacgtatgagtcggaggaagaggacaagtgcaagcctatgtcctatgaggagaagcggcagctcagcttggacatcaacaagctccccggcgagaagctgggccgcgtggtgcacatcatccagtcacgggagccctccctgaagaattccaaccccgacgagattgaaatcgactttgagaccctgaagccgtccacactgcgtgagctggagcgctatgtcacctcctgtttgcggaagaaaaggaaacctcaagctgagaaagttgatgtgattgccggctcctccaagatgaagggcttctcgtcctcagagtcggagagctccagtgagtccagctcctctgacagcgaagactccgaaacagagatggctccgaagtcaaaaaagaaggggcaccccgggagggagcagaagaagcaccatcatcaccaccatcagcagatgcagcaggccccggctcctgtgccccagcagccgcccccgcctccccagcagcccccaccgcctccacctccgcagcagcaacagcagccgccacccccgcctcccccaccctccatgccgcagcaggcagccccggcgatgaagtcctcgcccccacccttcattgccacccaggtgcccgtcctggagccccagctcccaggcagcgtctttgaccccatcggccacttcacccagcccatcctgcacctgccgcagcctgagctgccccctcacctgccccagccgcctgagcacagcactccaccccatctcaaccagcacgcagtggtctctcctccagctttgcacaacgcactaccccagcagccatcacggcccagcaaccgagccgctgccctgcctcccaagcccgcccggcccccagccgtgtcaccagccttgacccaaacacccctgctcccacagccccccatggcccaacccccccaagtgctgctggaggatgaagagccacctgccccacccctcacctccatgcagatgcagctgtacctgcagcagctgcagaaggtgcagccccctacgccgctactcccttccgtgaaggtgcagtcccagcccccaccccccctgccgcccccaccccacccctctgtgcagcagcagctgcagcagcagccgccaccacccccaccaccccagccccagcctccaccccagcagcagcatcagccccctccacggcccgtgcacttgcagcccatgcagttttccacccacatccaacagcccccgccaccccagggccagcagcccccccatccgcccccaggccagcagccacccccgccgcagcctgccaagcctcagcaagtcatccagcaccaccattcaccccggcaccacaagtcggacccctactcaaccggtcacctccgcgaagccccctccccgcttatgatacattccccccagatgtcacagttccagagcctgacccaccagtctccaccccagcaaaacgtccagcctaagaaacaggagctgcgtgctgcctccgtggtccagccccagcccctcgtggtggtgaaggaggagaagatccactcacccatcatccgcagcgagcccttcagcccctcgctgcggccggagccccccaagcacccggagagcatcaaggcccccgtccacctgccccagcggccggaaatgaagcctgtggatgtcgggaggcctgtgatccggcccccagagcagaacgcaccgccaccaggggcccctgacaaggacaaacagaaacaggagccgaagactccagttgcgcccaaaaaggacctgaaaatcaagaacatgggctcctgggccagcctagtgcagaagcatccgaccaccccctcctccacagccaagtcatccagcgacagcttcgagcagttccgccgcgccgctcgggagaaagaggagcgtgagaaggccctgaaggctcaggccgagcacgctgagaaggagaaggagcggctgcggcaggagcgcatgaggagccgagaggacgaggatgcgctggagcaggcccggcgggcccatgaggaggcacgtcggcgccaggagcagcagcagcagcagcgccaggagcaacagcagcagcagcaacagcaagcagctgcggtggctgccgccgccaccccacaggcccagagctcccagccccagtccatgctggaccagcagagggagttggcccggaagcgggagcaggagcgaagacgccgggaagccatggcagctaccattgacatgaatttccagagtgatctattgtcaatatttgaagaaaatcttttctga

SEQ ID NO: 2 - protein, 1362 aa, Homo sapiens
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKYQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 3 - DNA, 6000 bp, Mus musculus
ggaggaggaagcgactgcctctggatctgcggtgcgcgccgggccgcccaacaagatagctgtgttccagttttgttcaaaactactagtgaagaagtcctcctctcctctcattcccatatctgctttccgtctggacacaacattgggtctaagaaaatcagctcaccaggctgtgaccactgaatcacattggagttctctgtaagtgcctggtgaagaatgtgatgggatcactagcatgtctacggagagcggccctgggacaagattgagaaatctgccagtaatgggggatggactagaaacctcccaaatgtctacaacgcaggcccaggcccaaccccagccagcaaatgcagccagcaccaatcctccacccccagagacctccaaccctaacaagcccaagagacagacaaaccaactgcaatatctgctcagagtggtgctcaagacactatggaaacaccagtttgcgtggcctttccagcagcccgtggatgccgtcaagctgaacctccctgattactataagattattaaaacacccatggatatgggaacaataaagaagcgcttggaaaacaactattactggaatgctcaggaatgtatccaggacttcaacactatgtttacaaattgttacatctataacaagcctggagatgacatcgtcttaatggcagaagctctggagaagctcttcttgcaaaaaatcaatgaactgcctacagaagaaactgagatcatgatagtccaggcaaaaggaagaggacgagggaggaaagaaacaggggcagcaaagcctggtgtatccacggtaccaaacacaactcaagcatcaacttctccgcagacccagacgcctcagcagaaccctcctccacctgtgcaggccacaactcacccctttcctgctgtcaccccagacctcattgcccagcctcctgtcatgacaatggtgccccctcagccacttcagactccttcaccggtacccccccagccaccacccccacctgctccagttccacagcctgtgcagagtcacccgcccatcattgcgaccaccccccagcctgtgaagacaaagaaaggggtgaagaggaaagcagataccaccacccctaccaccatcgaccccattcatgagccaccctcactggccccagagcccaagaccgccaagctgggtcctcggcgggagagcagcagacctgtgaagcctccaaagaaggatgtaccggactcacagcagcacccagggccagagaagagcagcaagatctctgagcagctaaagtgctgcagtggcatcctcaaggagatgtttgccaagaaacatgctgcctatgcctggcctttctacaagcctgtggatgtggaggcactgggtctgcacgactactgtgacatcatcaaacatcccatggacatgagcacaatcaagtctaaactagagtcccgagagtacagagatgcccaggaatttggtgctgatgtccgattgatgttctccaactgctacaagtacaacccccctgaccatgaagtggtagccatggctcgaaaactccaggatgtgtttgaaatgcgctttgccaagatgcctgatgagcctgaagagccagttgttacagtgtcctctcctgcagtgccaccccctacaaaggtggtagccccaccctcatctagtgacagcagcagcgacagttcttccgacagcgacagttccactgacgactctgaggaagagcgagcccagcggctggctgaactccaggaacagctcaaggccgtgcatgagcagcttgcagccctctcacagccccagcagaacaaaccaaagaaaaaggagaaggacaagaaggaaaagaaaaaggaaaagcacaaaaagaaagaagaagtggaggaaaataaaaaaagcaaaaccaaggaacttcctcccaaaaagacaaagaaaaataacagcagcaacagcaatgtgagcaagaaggaaccagtacccacgaagaccaagccgcctcccacatatgaatcagaagaggaggataagtgtaagcccatgtcttatgaggagaagcggcagctaagtctagatatcaacaaacttcctggtgagaagctaggccgtgtagtacacataattcagtcaagggaaccatcacttaaaaactccaaccccgatgagattgagattgactttgagaccctgaagccatctacactacgagagttggagcgatatgtcacctcctgtttgcggaagaaaaggaaacctcaagctgaaaaagttgacgtgattgctggttcttccaagatgaagggattctcatcctctgagtcggagagcaccagcgaatccagctcctctgacagtgaagactctgaaacagagatggctcccaagtcaaaaaagaaggggcacactgggagggaccagaaaaagcatcatcaccatcaccatccacagatgcagccagccccagctcctgtgccccagcagccgcccccacctccacagcagcctcctccacccccacctccgcagcagcagcagcagcaacctccacccccaccacctccgccctccatgccacagcagactgccccagcgatgaagtcctcgcccccacccttcatcactgcccaggtccccgtcctggaaccacagctgccaggcagtgtctttgaccctattagccacttcacccagcccatcttgcacctgccgcagccggagctgcctcctcacctgccccagccacctgagcacagcactccaccccatctcaaccagcatgctgtggtctctcctccagctttgcacaatgcgctgccccaacagccatctcggcccagtaaccgagctgctgctctgcccccaaagcctacccgacccccagctgtgtcccctgccctggcccagccccccctgctcccacaaccaccaatggctcagcccccccaagtgctgctggaggatgaagagccacctgccccacccctcacctccatgcagatgcagttgtacctacagcagctgcagaaggtgcagcctcccacaccactactcccttccgtgaaggtgcagtcccaacccccaccgcctttgccgcctccaccccacccttctgtgcagcagcagcagctccagccacagccaccgccacccccacctcctcagccacagccaccaccccagcaacaacaccagcctccaccacgaccagttcacttgccatccatgcccttttctgctcatattcagcagcccccaccacccccaggacagcaacctactcacccacccccaggacagcagcccccaccaccacagcctgccaagccccagcaagtcatccagcatcacccttccccccggcaccacaagtcagacccctactcagctggtcatcttcgtgaggctccctctccgcttatgatacattcccctcagatgccacagttccagagcctgacccatcagtctcctccccagcaaaacgtccagcctaagaagcaggtaaagggcagggctgagccacagccaccagggccagtcatgggccaaggccagggatgcccacctgcctcaccggctgccgtgcctatgctgtcccaggagctgcgaccaccttcagtcgtccagccccagcccctggtggtagtaaaggaggagaagattcactcaccaatcattcgcagcgagcctttcagcacctcacttcgaccagagccccccaagcacccagagaacatcaaggccccagtccacctgccccagcggcctgagatgaagcctgtagacatagggaggcctgtgatccggcctccagagcagagtgcacccccaccaggggcccctgacaaggacaaacagaagcaggagccaaaaacaccagtggcacccaaaaaggacctgaaaattaagaacatgggctcctgggccagcctggtacagaagcatccgaccaccccatcctccacagccaagtcctcaagtgacagctttgagcatttccgccgtgctgctcgggagaaggaggagagggaaaaggccctgaaggctcaggctgagcatgcagagaaggagaaggagcggctgaggcaggagcgcatgaggagccgagaggatgaagatgcgctggaacaggcacgtcgagcccatgaggaggcacgacggcgccaggagcagcagcagcagcagcagcagcagagacaggagcagcagcagcagcagcagcaggcagctgctgtggccgctgcctctgccccccaggcccagagctcccagccccagtctatgctggaccagcagagggagttggcccggaaacgagagcaggagcggaggcgcagggaggcaatggcagctacaattgacatgaatttccagagtgatcttttgtcaatatttgaagaaaatcttttttgagagcacccaggtgacttctgactttttctggcaaaaatgttgactctccatccatagtgttaggggcagccgtggaagcagcagcagccagggatgtctctgggcctggctctcctgcatgctgtgcccggggcaggcctgacgggcgggcagctgaggatcgcagagcccatctgccttacagccagccggacagacgtcctactacatgccaccccctcagaggacgtcagttcagggcacgtctcaatatgagtcaagtgccagctggacctgaggcctgcttccccgcgtgcggggcagaggcccttctctgcgccaaatgtctgactacacagtatacacaggacattgttgctgccgccaccgagactggttttctgtccctgagaacatgacgtttgtgacgtcctacccattgggagtccttcacaccccagccatcgctgcccactcccaggaggccagggcaagcctgtgtggactggagcagcaaggcactggcccaccccttgctggcactgactttgccttgaacagaccctctcccctcccccacaagcctttaattgagagccgctctttataagtgttcgcttgtgcaaaagggaatagtgccgtggaagtgtgtgtccatggcagtttggacaggggtgactgttcacacgtatggacaggcctcgcctggggagtaaggccaccaaccaaagtcagttccttcccacctgtgtttctgattttgttttgttttatttttttcctatatatatattttttttgttgttgttgaattctattttatttttaactctcttctcctcagacacagtggcactgcttatctccaaatggtgtgatcgtctcctcagtgagcggcggctcccactgcgctgtgggtagtgtgtgactgtggctgtactgtatagtgaacatagttggcatatctttgtttgaagtttgttggtgattccaccaaactggtgtaaaaaacaaaaacaaaaaaacccacaaaccacccaaaactcacaaaaaaaatcctgcttgtattcagtttcaaactttattagtctcacttttaattataaaaccagaaagctgcaatttcttttccctctaccccttatttgttggcttctttgtttttttaatgtcaaatctgtttgtttttaccaagaaagggagagcctgaggaggtctgcaggctttggtgggctgcgaaccgaggcggaggcccccacccagtacctggtgagaccagtgtgctgcgccttccttcctattagtatttttgtttcgttttgtttttaaattggagcccctggtgaggttacatgtgccatgagaacccactctacaccatagtgctggtgcctcagtgttggccaaactctggagtcactgactggtttgactttcatgtggtaaatatgcatttggtctgtactgatcatggaataaacacatctctcttttttaaaaaaaaaaaaaaaaaaaaaaaaaaataaaacgaaaaaaaaaaaaaaaaaaaaaaaaaaaaaaaaaaaaaaaaaaaaaaa

SEQ ID NO: 4 - protein, 1400 aa, Mus musculus
MSTESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGAAKPGVSTVPNTTQASTSPQTQTPQQNPPPPVQATTHPFPAVTPDLIAQPPVMTMVPPQPLQTPSPVPPQPPPPPAPVPQPVQSHPPIIATTPQPVKTKKGVKRKADTTTPTTIDPIHEPPSLAPEPKTAKLGPRRESSRPVKPPKKDVPDSQQHPGPEKSSKISEQLKCCSGILKEMFAKKHAAYAWPFYKPVDVEALGLHDYCDIIKHPMDMSTIKSKLESREYRDAQEFGADVRLMFSNCYKYNPPDHEVVAMARKLQDVFEMRFAKMPDEPEEPVVTVSSPAVPPPTKVVAPPSSSDSSSDSSSDSDSSTDDSEEERAQRLAELQEQLKAVHEQLAALSQPQQNKPKKKEKDKKEKKKEKHKKKEEVEENKKSKTKELPPKKTKKNNSSNSNVSKKEPVPTKTKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESTSESSSSDSEDSETEMAPKSKKKGHTGRDQKKHHHHHHPQMQPAPAPVPQQPPPPPQQPPPPPPPQQQQQQPPPPPPPPSMPQQTAPAMKSSPPPFITAQVPVLEPQLPGSVFDPIGHFTQPILHLPQPELPPHLPQPPEHSTPPHLNQHAVVSPPALHNALPQQPSRPSNRAAALPPKPTRPPAVSPALAQPPLLPQPPMVQPPQVLLEDEEPPAPPLTSMQMQLYLQQLQKVQPPTPLLPSVKYQSQPPPPLPPPPHPSVQQQQLQPQPPPPPPPQPQPPPQQQHQPPPRPVHLPSMPFSAHIQQPPPPPGQQPTHPPPGQQPPPPQPAKPQQVIQHHPSPRHHKSDPYSAGHLREAPSPLMIHSPQMPQFQSLTHQSPPQQNVQPKKQVKGRAEPQPPGPVMGQGQGCPPASPAAVPMLSQELRPPSVVQPQPLVVVKEEKIHSPIIRSEPFSTSLRPEPPKHPENIKAPVHLPQRPEMKPVDIGRPVIRPPEQSAPPPGAPDKDKQKQEPKTPVAPKKDLKIKNMGSWASLVQKHPTTPSSTAKSSSDSFEHFRRAAREKEEREKALKAQAEHAEKEKERLRQERMRSREDEDALEQARRAHEEARRRQEQQQQQQQQRQEQQQQQQQAAAVAAASAPQAQSSQPQSMLDQQRELARKREQERRRREAMAATIDMNFQSDLLSIFEENLF

SEQ ID NO: 5 - DNA, 12 bp, Artificial Sequence
  Description of Artificial Sequence Synthetic oligonucleotide
aaaacaaaaaaa

SEQ ID NO: 6 - DNA, 12 bp, Artificial Sequence
  Description of Artificial Sequence Synthetic oligonucleotide
accnnnnnnggt

SEQ ID NO: 7 - DNA, 19 bp, Artificial Sequence
  Synthetic oligonucleotide
gacactatggaaacaccag

SEQ ID NO: 8 - DNA, 19 bp, Artificial Sequence
  Synthetic oligonucleotide
gcgggagcaggagcgaaga

SEQ ID NO: 9 - DNA, 20 bp, Artificial Sequence
  Synthetic oligonucleotide
gcaagctgaccctgaagttc

(19) United States
(12) Patent Application Publication
(10) Pub. No.: US 2006/0223055 A1
(43) Pub. Date: Oct. 5, 2006

(54) Methods and compositions for treatment of viral lnfection

(76) Inventors: Peter M. Howley, Wellesley, MA (US); Jianxin You, Brookline, MA (US)

Correspondence Address: FOLEY HOAG, LLP;PATENT GROUP, WORLD TRADE CENTER WEST, 155 SEAPORT BLVD., BOSTON, MA, 02210-2600, US

(21) Appl. No.: 11/322,164

(22) Filed: Dec. 29, 2005

Related U.S. Application Data
(63) Continuation-in-part of application No. PCT/US04/21477, filed on Jul. 1, 2004.
(60) Provisional application No. 60/484,417, filed on Jul. 1, 2003.
(60) Provisional application No. 60/484,792, filed on Jul. 3, 2003.

(51) Int. Cl. C12Q 1/70; C07H 21/04; C07K 14/03; C07K 16/08; C12N 15/86

(52) U.S. Cl. 435/5; 435/69.3; 435/325; 435/456; 530/350; 530/388.3; 536/23.72

(57) ABSTRACT

The present invention relates to methods and compositions for the treatment and/or prevention of diseases or disorders associated with viral infection. Accordingly, the nucleotide and amino acid sequences for a human Brd4 protein are provided. Also provided are complexes comprising Brd4 and an E2 protein or a functional equivalent of an E2 protein.

### RELATED APPLICATIONS

This application is a continuation-in-part of PCT application number PCT/US04/21477, filed on Jul. 1, 2004, which claims the benefit of priority to U.S. Provisional Patent Application No. 60/484,417, filed on Jul. 1, 2003, and U.S. Provisional Patent Application No. 60/484,792, filed Jul. 3, 2003, which applications are hereby incorporated by reference in their entirety.

### GOVERNMENT SUPPORT

This invention was made with government support under Grant number CA077385 awarded by the National Institutes of Health. The government has certain rights in this invention.

### BACKGROUND

Illnesses resulting from viral infections remain a major problem to be addressed by modern medicine. To date, there is no suitably effective treatment for infection with any known virus. Recently there has been progress towards treatment of several viral diseases, however, these treatments are largely directed at specific viruses or classes of viruses. For example, protease inhibitors targeting the virally-encoded human immunodeficiency virus (HIV) protease have been effective against some strains of HIV, and have been responsible, when used in combination with other anti-viral agents, for a decline in HIV-related deaths in the United States. However, the protease inhibitors are directed to specific viruses or classes of viruses, and are not useful for the treatment of viruses outside of those classes. In addition, there is evidence that strains resistant to these new agents are evolving.

It is noted that the virus-specific agents currently being used in developed countries are very expensive, being beyond the means of a great number of infected individuals throughout the world. In addition, the dosage regimens are complex and demand careful attention by physicians and the infected individuals.

Because viruses co-opt the host's own normal intracellular metabolic processes for their reproductive needs, a major difficulty in the design of antiviral agents is to make agents that target the virus without toxicity to the host organism.

There is a need in the art for antiviral agents that are effective against a broad spectrum of viruses, relatively non-toxic, inexpensive to produce, and simple to administer.

### SUMMARY

The present disclosure provides methods and compositions for treatment and/or prevention of viral infections.

In one aspect, the invention provides isolated Brd4 polypeptides, comprising: (a) an amino acid sequence set forth in SEQ ID NO: 2; (b) an amino acid sequence having at least 95% identity with the amino acid sequence set forth in SEQ ID NO: 2; or (c) an amino acid sequence encoded by a polynucleotide that hybridizes under stringent conditions to the complementary strand of a polynucleotide having SEQ ID NO: 1 and wherein said polypeptide has at least one biological activity of a Brd4 protein.

In certain embodiments, the Brd4 polypeptides are capable of interacting with an E2 protein or a functional equivalent of an E2 protein. In an exemplary embodiment, the Brd4 polypeptides are capable of interacting with the transactivation domain of an E2 protein or a functional equivalent of an E2 protein.

In one embodiment, the invention provides an isolated polypeptide comprising at least 5, 10, 20, 25, 30, 40, 50, or more, consecutive amino acid residues of SEQ ID NO: 2 wherein said polypeptide is capable of disrupting an interaction between a Brd4 protein and an E2 protein or a functional equivalent of an E2 protein. In exemplary embodiments, the invention provides isolated polypeptides comprising at least 5, 10, 20, 25, 30, 40, 50, or more, consecutive amino acid residues of a region of SEQ ID NO: 2 having amino acids 1047-1362 or a region of SEQ ID NO: 2 having amino acids 1224-1362. In one embodiment, a polypeptide comprising amino acid residues 1047-1362 of SEQ ID NO: 2 is provided. In another embodiment, a polypeptide comprising amino acid residues 1224-1362 of SEQ ID NO: 2 is provided. In still other embodiments, the invention provides peptidomimetics based on the sequence set forth in SEQ ID NO: 2 or a fragment thereof.

In another embodiment, the invention provides an isolated monoclonal antibody that binds to a polypeptide comprising SEQ ID NO: 2 and does not bind to a polypeptide comprising SEQ ID NO: 4. In another embodiment, the antibody binds specifically to a polypeptide comprising SEQ ID NO: 2. In yet another embodiment, the invention provides an anti-human Brd4 antibody that does not substantially cross-react (e.g., less than 50%, 40%, 30%, 25%, 20%, 15%, 10%, 5%, 2%, 1%, 0.5%, 0.1%, or less cross-reactivity) with a protein which is less than 95% identical to SEQ ID NO: 2. In various embodiments, such antibodies may be single chain and/or humanized antibodies. In other embodiments, the antibodies of the invention may be formulated in a pharmaceutically acceptable carrier. In one embodiment, the invention provides an antibody that interacts with a portion of a Brd4 protein that interacts with an E2 protein or a functional equivalent of an E2 protein. In another embodiment, the invention provides an antibody that interacts with a region of human Brd4 that comprises amino acid residues 1047-1362 or residues 1224-1362 of SEQ ID NO: 2.

In another aspect, the invention provides an isolated nucleic acid comprising (a) the nucleotide sequence of SEQ ID NO: 1, (b) a nucleotide sequence at least 90% identical to SEQ ID NO: 1, (c) a nucleotide sequence that hybridizes under stringent conditions to SEQ ID NO: 1, or (d) the complement of the nucleotide sequence of (a), (b) or (c).

In yet another aspect, the invention provides an isolated nucleic acid comprising a nucleotide sequence encoding a fragment of SEQ ID NO: 2 wherein said fragment comprises at least 5 consecutive amino acid residues and wherein said fragment is capable of disrupting an interaction between a Brd4 protein and an E2 protein or a functional equivalent of an E2 protein.

In various embodiments, the nucleic acids described herein may further comprise a transcriptional regulatory sequence operably linked to said nucleotide sequence so as to render said nucleic acid suitable for use in an expression vector. Additionally, the nucleotide sequences described herein may be contained on a vector, such as, for example, an expression vector.

In another aspect, the invention provides a host cell comprising a nucleic acid encoding a polypeptide comprising (a) an amino acid sequence set forth in SEQ ID NO: 2; (b) an amino acid sequence having at least 95% identity with the amino acid sequence set forth in SEQ ID NO: 2; or (c) an amino acid sequence encoded by a polynucleotide that hybridizes under stringent conditions to the complementary strand of a polynucleotide having SEQ ID NO: 1 and wherein said polypeptide has at least one biological activity of a Brd4 protein.

In yet another aspect, the invention provides an isolated complex comprising (a) Brd4 and an E2 polypeptide or a functional equivalent of an E2 polypeptide; (b) Brd4 and a fragment of an E2 polypeptide or a functional equivalent of an E2 polypeptide; (c) a fragment of Brd4 and an E2 polypeptide or a functional equivalent of an E2 polypeptide; or (d) a fragment of Brd4 and a fragment of an E2 polypeptide or a functional equivalent of an E2 polypeptide. In one exemplary embodiment, the invention provides a complex comprising (a) an amino acid sequence set forth in SEQ ID NO: 2; (b) an amino acid sequence having at least 95% identity with the amino acid sequence set forth in SEQ ID NO: 2; or (c) an amino acid sequence encoded by a polynucleotide that hybridizes under stringent conditions to the complementary strand of a polynucleotide having SEQ ID NO: 1 and wherein said polypeptide has at least one biological activity of a Brd4 protein. In another exemplary embodiment, the invention provides a complex comprising an E2 protein from a papilloma virus, including a human papilloma virus (HPV) or a non-human animal papillomavirus (such as, for example, a bovine papilloma virus (BPV), a canine papillomavirus, a feline papillomavirus, a monkey papillomavirus, an equine papillomavirus, etc.), or a functional equivalent of an E2 protein from a herpes virus. In another exemplary embodiment, the invention provides a complex comprising a latency-associated nuclear antigen (LANA) protein from a Kaposi sarcoma-associated herpesvirus (KSHV).

In another embodiment, the invention provides an isolated antibody that has a higher binding affinity for a complex of claim 24 than for the individual polypeptides of said complex. In an exemplary embodiment, the invention provides an antiobdy that disrupts, or inhibits the formation of, a complex comprising a Brd4 protein and an E2 protein or a functional equivalent of an E2 protein.

In another aspect, the invention provides a fusion polypeptide, comprising an amino acid sequence having: (a) the amino acid sequence set forth in SEQ ID NO: 2; (b) an amino acid sequence having at least 95% identity with the amino acid sequence set forth in SEQ ID NO: 2; (c) an amino acid sequence encoded by a polynucleotide that hybridizes under stringent conditions to the complementary strand of a polynucleotide having SEQ ID NO: 1 and wherein said polypeptide has at least one biological activity of a Brd4 protein; or (d) an amino acid sequence having at least five consecutive amino acid residues of SEQ ID NO: 2 wherein said polypeptide is capable of interacting with an E2 protein or a functional equivalent of an E2 protein; fused to a polypeptide selected from the group consisting of: (e) an E2 polypeptide; (f) a functional equivalent of an E2 polypeptide; or (g) a fragment of (e) or (f) that is capable of interacting with a polypeptide of (a), (b), (c), or (d).

In another aspect, the invention provides a method for identifying a compound that disrupts a Brd4 protein complex, comprising:

(i) providing a reaction mixture comprising (a) Brd4 and an E2 polypeptide or a functional equivalent of an E2 polypeptide; (b) Brd4 and a fragment of an E2 polypeptide or a functional equivalent of an E2 polypeptide; (c) a fragment of Brd4 and an E2 polypeptide or a functional equivalent of an E2 polypeptide; or (d) a fragment of Brd4 and a fragment of an E2 polypeptide or a functional equivalent of an E2 polypeptide;

(ii) contacting the reaction mixture with a test agent; and

(iii) determining the effect of the test agent on the formation or stability of a complex comprising (a), (b), (c), or (d), wherein a decrease in the formation or stability of said complex is indicative of a compound that disrupts a Brd4 protein complex.

In an exemplary embodiment, the reaction mixture is a cell or cell population which may optionally be infected with a virus or otherwise contain at least a portion of a viral genome.

In another aspect, the invention provides a method for identifying modulators of a Brd4 protein complex, comprising:

(i) forming a reaction comprising a complex, wherein said complex comprises: (a) Brd4 and an E2 polypeptide or a functional equivalent of an E2 polypeptide; (b) Brd4 and a fragment of an E2 polypeptide or a functional equivalent of an E2 polypeptide; (c) a fragment of Brd4 and an E2 polypeptide or a functional equivalent of an E2 polypeptide; or (d) a fragment of Brd4 and a fragment of an E2 polypeptide or a functional equivalent of an E2 polypeptide;

(ii) contacting the reaction mixture with a test agent; and

(iii) determining the effect of the test agent on one or more of the following activities: (a) a change in the level of said complex, (b) a change in the activity of said complex, (c) a change in the stability of said complex, (d) a change in the conformation of said complex, (e) a change in the activity of at least one polypeptide of said complex, (f) a change in the conformation of at least one polypeptide of said complex, (g) where the reaction mixture is a whole cell, a change in the intracellular localization of the complex or a component thereof, (h) where the reaction mixture is a whole cell, a change in the transcription level of a gene dependent on the complex, and (i) where the reaction mixture is a whole cell, a change in second messenger levels in the cell.

In another aspect, the invention provides a method for identifying a compound that inhibits viral infectivity or proliferation comprising:

(i) providing a reaction mixture comprising (a) Brd4 and an E2 polypeptide or a functional equivalent of an E2 polypeptide; (b) Brd4 and a fragment of an E2 polypeptide or a functional equivalent of an E2 polypeptide; (c) a fragment of Brd4 and an E2 polypeptide or a functional equivalent of an E2 polypeptide; or (d) a fragment of Brd4 and a fragment of an E2 polypeptide or a functional equivalent of an E2 polypeptide;

(ii) contacting the reaction mixture with a test agent; and

(iii) determining the effect of the test agent on the formation or stability of a complex comprising (a), (b), (c), or (d), wherein a decrease in the formation or stability of said complex is indicative of a compound that inhibits viral infectivity or proliferation.

In another aspect, the invention provides a method for treating a subject suffering from a viral related disease or disorder, comprising administering to an animal having said condition a therapeutically effective amount of a polypeptide comprising at least five consecutive amino acids from a region of SEQ ID NO: 2 having amino acids 1224-1362, wherein said polypeptide is capable of binding to an E2 polypeptide or a functional equivalent of an E2 polypeptide. In certain embodiment, the subject may be suffering from a disease or disorder related to an infection of a papillomavirus, herpes virus, Epstein Barr virus, or a Kaposi sarcoma-associated virus. In various embodiments, the methods and compositions described herein may be used to treat any organism which is susceptible to a viral infection, including, for example, plants and animals. In an exemplary embodiment, the methods and compositions described herein may be used to treat a human. In other embodiments, the methods and compositions described herein may be used to treat a livestock animal, such as, for example, a cow, pig, goat or sheep.

In another aspect, the invention provides a method for inhibiting Brd4 dependent growth or infectivity of a virus, comprising contacting a virus infected cell with a polypeptide comprising at least five consecutive amino acids from a region of SEQ ID NO: 2 having amino acids 1224-1362, wherein said polypeptide is capable of binding to an E2 polypeptide or a functional equivalent of an E2 polypeptide.

### BRIEF DESCRIPTION OF THE DRAWINGS

FIG. 1 shows a schematic of the cloning of Human Brd4 (HBrd4).

FIG. 2 shows an alignment between the amino acid sequences for human Brd4 (SEQ ID NO: 2) and mouse Brd4 (SEQ ID NO: 4).

FIG. 3 shows the results of experiments carried out to map the E2 binding domain on human Brd4 protein.

FIG. 4. Binding of HPV16 E2 transactivation domain mutants to Brd4 C-terminal domain. The bound ³⁵S-labelled Brd4-CTD was quantified with a Phosphoimager, setting E2 (wt) binding as 100%.

FIG. 5. Model of the HPV16 E2 transactivation domain (PDB: 1DTO) was visualized with the Swiss-PdbViewer program (16). Shown are the two opposite surfaces of the TA domain. Amino acids important for Brd4 binding (FIG. 4) and transcriptional activation, but not E1 binding are indicated in red. Residues important for E1 binding, but neither Brd4 binding nor the transactivation function are shown in blue. In purple are amino acids for which mutants defective for all E2 functions as summarized in Table 2.

### DETAILED DESCRIPTION

General

The papillomavirus E2 protein is a multifunctional viral gene product that has been implicated in viral DNA replication, viral transcription, and regulation of cellular transformation. In addition, E2 protein has been shown to play a critical role in plasmid maintenance by linking the viral genomes to the cellular mitotic chromosomes to ensure their accurate segregation into daughter cells. To identify cellular factors that may play important roles in E2 virus-host cell interactions, we employed a proteomic tandem affinity purification (TAP) approach to systematically analyze cellular proteins that associate with E2 in vivo. Mass spec analysis of the proteins co-purified with E2 has identified the Brd4 protein as a factor that associates with the viral E2 protein.

Using co-immunoprecipitation, we showed that endogenous Brd4 interacts with both human and bovine papillomavirus E2 protein, suggesting a conserved role involving Brd4 in papillomavirus E2 function. Brd4 interacts specifically with the N-terminal transactivation domain of E2, and the E2 binding region on Brd4 has been mapped to its C-terminal region. Immunofluorescent analysis revealed the co-localization of E2 and Brd4 on mitotic chromosomes in human cells. Expression of a truncated C-terminal domain of Brd4 inhibits the interaction of endogenous Brd4 with E2 and also prevents the co-localization of the viral protein and its cellular partner. Co-transfection of this dominant-negative truncation mutant of Brd4 with BPV-1 genome into C127 cells significantly inhibited the transformation efficiency. Taken together, our studies indicate that the cellular protein Brd4 is an important therapeutic target for papillomavirus infections.

Polypeptides

The present invention makes available in a variety of embodiments soluble, purified and/or isolated forms of human Brd4 polypeptides and complexes comprising human Brd4 polypeptides.

In one aspect, the present invention contemplates an isolated polypeptide comprising (a) the amino acid sequence set forth in SEQ ID NO: 2 or an amino acid sequence having residues 1047-1362, 1134-1362, or 1224-1362 of SEQ ID NO: 2, (b) an amino acid sequence of (a) with 1 to about 20 conservative amino acid substitutions, deletions, or additions, (c) an amino acid sequence that is at least 95% identical to an amino acid sequence of (a), or (d) a functional fragment of a polypeptide having an amino acid sequence set forth in (a), (b) or (c). In an exemplary embodiment, the invention contemplates polypeptides having at least 3, 5, 7, 10, 15, 20, 25, 30, 40, 45, 50, 75, 100, 150, 200, 300, 500, or more consecutive amino acids from SEQ ID NO: 2 or a region of SEQ ID NO: 2 having amino acid residues 1047-1362, 1134-1362, or 1224-1362 of SEQ ID NO: 2.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 85 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis and mass-spectrometry analysis.

In another aspect, the present invention contemplates a complex comprising (a) human Brd4 and an E2 polypeptide or a functional equivalent of an E2 polypeptide; (b) human Brd4 and a fragment of an E2 polypeptide or a functional equivalent of an E2 polypeptide; (c) a fragment of human Brd4 and an E2 polypeptide or a functional equivalent of an E2 polypeptide; or (d) a fragment of human Brd4 and a fragment of an E2 polypeptide or a functional equivalent of an E2 polypeptide. In one embodiment, the complex comprises a fragment of Brd4 having residues 1047-1362, 1134-1362, or 1224-1362 of SEQ ID NO: 2. In another embodiment, the complex comprises a fragment having at least five consecutive amino acid residues from SEQ ID NO: 2 or a region of SEQ ID NO: 2 having amino acid residues 1047-1362, 1134-1362, or 1224-1362 of SEQ ID NO: 2.

In certain embodiments, a polypeptide of the invention comprises one or more post-translational or chemical modifications modifications. Exemplary modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

In another aspect, the present invention contemplates a polypeptide of the invention contained within a syringe (or other device for, e.g., introducing the polypeptide into a subject) or bound to a solid support. Exemplary solid supports include the following: particles, strands, precipitates, gels, sheets, tubing, spheres, containers, capillaries, pads, slices, films, plates and slides.

In certain embodiments, a polypeptide of the invention is a fusion protein containing a domain which increases its solubility and/or facilitates its purification, identification, detection, and/or structural characterization. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, a polypeptide of the invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope of the invention to include linker sequences between a polypeptide of the invention and the fusion domain in order to facilitate construction of the fusion protein or to optimize protein expression or structural constraints of the fusion protein. In another embodiment, the polypeptide may be constructed so as to contain protease cleavage sites between the fusion polypeptide and polypeptide of the invention in order to remove the tag after protein expression or thereafter. Examples of suitable endoproteases, include, for example, Factor Xa and TEV proteases.

In another embodiment, a polypeptide of the invention may be modified so that its rate of traversing the cellular membrane is increased. For example, the polypeptide may be fused to a second peptide which promotes “transcytosis,” e.g., uptake of the peptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell_{—}55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is coupled. Thus, polypeptides may be fused to a peptide consisting of about amino acids 42-58 of Drosophila antennapedia or shorter fragments for transcytosis (Derossi et al. (1996) J Biol Chem 271:18188-18193; Derossi et al. (1994) J Biol Chem 269:10444-10450; and Perez et al. (1992) J Cell Sci 102:717-722). The transcytosis polypeptide may also be a non-naturally-occurring membrane-translocating sequence (MTS), such as the peptide sequences disclosed in U.S. Pat. No. 6,248,558.

In still another embodiment, the polypeptides of the invention are labeled to facilitate their detection, purification, and/or structural characterization. Exemplary labels include, for example, radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). In an exemplary embodiment, a polypeptide of the invention is fused to a heterologous polypeptide sequence which produces a detectable fluorescent signal, including, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

In other embodiments, the invention provides for polypeptides of the invention immobilized onto a solid surface, including, plates, microtiter plates, slides, beads, particles, spheres, films, strands, precipitates, gels, sheets, tubing, containers, capillaries, pads, slices, etc. The polypeptides of the invention may be immobilized onto a “chip” as part of an array. An array, having a plurality of addresses, may comprise one or more polypeptides of the invention in one or more of those addresses. In one embodiment, the chip comprises one or more polypeptides of the invention as part of an array of mammalian and/or viral polypeptide sequences.

In still other embodiments, the invention comprises the polypeptide sequences of the invention in computer readable format. The invention also encompasses a database comprising the polypeptide sequences of the invention.

In other embodiments, the invention relates to the polypeptides of the invention contained within a vessels useful for manipulation of the polypeptide sample. For example, the polypeptides of the invention may be contained within a microtiter plate to facilitate detection, screening or purification of the polypeptide. The polypeptides may also be contained within a syringe as a container suitable for administering the polypeptide to a subject in order to generate antibodies or as part of a vaccination regimen. The polypeptides may also be contained within an NMR tube in order to enable characterization by nuclear magnetic resonance techniques.

It is also possible to modify the structure of the subject proteins for such purposes as enhancing therapeutic or prophylactic efficacy, or stability (e.g., ex vivo shelf life, resistance to proteolytic degradation in vivo, etc.). Such modified polypeptides may be produced, for instance, by amino acid substitution, deletion, or addition, which substitutions may consist in whole or part by conservative amino acid substitutions.

For instance, it is reasonable to expect that an isolated conservative amino acid substitution, such as replacement of a leucine with an isoleucine or valine, an aspartate with a glutamate, a threonine with a serine, will not have a major affect on the biological activity of the resulting molecule. Whether a change in the amino acid sequence of a polypeptide results in a functional homolog may be readily determined by assessing the ability of the variant polypeptide to produce a response similar to that of the wild-type protein. Polypeptides in which more than one replacement has taken place may readily be tested in the same manner.

Polypeptides containing modified amino acids are also included. Examples of modified amino acids include analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) or (L) stereoisomers. D- and L-α-Amino acids are represented by the following Fischer projections and wedge-and-dash drawings. In the majority of cases, D- and L-amino acids have R- and S-absolute configurations, respectively.

Peptides and Peptidomimetics

In certain embodiments, the invention provides modified peptides that retain the ability to form a complex with a Brd4 protein, an E2 protein, or a functional equivalent of an E2 protein. Such modifications include N-terminal acetylation, glycosylation, biotinylation, etc.

Peptides with an N-Terminal D-Amino Acid. The presence of an N-terminal D-amino acid increases the serum stability of a peptide which otherwise contains L-amino acids, because exopeptidases acting on the N-terminal residue cannot utilize a D-amino acid as a substrate (Powell, et al. (1993), cited above). Thus, the amino acid sequences of the peptides with N-terminal D-amino acids are usually identical to the sequences of the L-amino acid peptides except that the N-terminal residue is a D-amino acid.

Peptides with a C-Terminal D-Amino Acid. The presence of a C-terminal D-amino acid also stabilizes a peptide, which otherwise contains L-amino acids, because serum exopeptidases acting on the C-terminal residue cannot utilize a D-amino acid as a substrate (Powell, et al. (1993), cited above). Thus, the amino acid sequences of the these peptides are usually identical to the sequences of the L-amino acid peptides except that the C-terminal residue is a D-amino acid.

Cyclic Peptides. Cyclic peptides have no free N- or C-termini. Thus, they are not susceptible to proteolysis by exopeptidases, although they are of course susceptible to endopeptidases, which do not cleave at peptide termini. The amino acid sequences of the cyclic peptides may be identical to the sequences of the L-amino acid peptides except that the topology is circular, rather than linear.

Peptides with Substitution of Natural Amino Acids by Unnatural Amino Acids. Substitution of unnatural amino acids for natural amino acids can also confer resistance to proteolysis. Such a substitution can, for example, confer resistance to proteolysis by exopeptidases acting on the N-terminus. For example, a serine residue can be substituted by a beta-amino acid isoserine. Such substitutions have been described (Coller, et al. (1993), J. Biol. Chem., 268:20741-20743) and these substitutions do not affect biological activity. Furthermore, the synthesis of peptides with unnatural amino acids is routine and known in the art (see, for example, Coller, et al. (1993)).

Peptides with N-Terminal or C-Terminal Chemical Groups. An effective approach to confer resistance to peptidases acting on the N-terminal or C-terminal residues of a peptide is to add chemical groups at the peptide termini, such that the modified peptide is no longer a substrate for the peptidase. One such chemical modification is glycosylation of the peptides at either or both termini. Certain chemical modifications, in particular N-terminal glycosylation, have been shown to increase the stability of peptides in human serum (Powell et al. (1993), Pharma. Res., 10: 1268-1273). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

Reverse-D Peptides. In another embodiment of this invention the peptides are reverse-D peptides. The term “reverse-D peptide” refers to peptides containing D-amino acids, arranged in a reverse sequence relative to a peptide containing L-amino acids. Thus, the C-terminal residue of an L-amino acid peptide becomes N-terminal for the D-amino acid peptide, and so forth. Reverse-D peptides retain the same tertiary conformation, and therefore the same activity, as the L-amino acid peptides, but are more stable to enzymatic degradation in vitro and in vivo, and thus have greater therapeutic efficacy than the original peptide (Brady and Dodson (1994), Nature, 368: 692-693; Jameson et al. (1994), Nature, 368: 744-746).

A “reversed” or “retro” peptide sequence as disclosed herein refers to that part of an overall sequence of covalently-bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman, M. and Chorev, M. Accounts of Chem. Res. 1979, 12, 423.

The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed peptide compounds of the invention can be formed by utilizing an appropriate amino acid mimetic moiety to link the two adjacent portions of the sequences depicted above utilizing a reversed peptide (reversed amide) bond. In case (a) above, a central residue of a diketo compound may conveniently be utilized to link structures with two amide bonds to achieve a peptidomimetic structure. In case (b) above, a central residue of a diamino compound will likewise be useful to link structures with two amide bonds to form a peptidomimetic structure.

The reversed direction of bonding in such compounds will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is preferably (D), and the configuration of the non-reversed portion is preferably (L). Opposite or mixed configurations are acceptable when appropriate to optimize a binding activity. The peptides of this invention, including the analogs and other modified variants, may generally be prepared following known techniques. Preferably, synthetic production of the peptide of the invention may be according to the solid phase synthetic method. For example, the solid phase synthesis is well understood and is a common method for preparation of peptides, as are a variety of modifications of that technique (Merrifield (1964), J. Am. Chem. Soc., 85: 2149; Stewart and Young (1984), Solid Phase Peptide Synthesis, Pierce Chemical Company, Rockford, Ill.; Bodansky and Bodanszky (1984), The Practice of Peptide Synthesis, Springer-Verlag, New York; Atherton and Sheppard (1989), Solid Phase Peptide Synthesis: A Practical Approach, IRL Press, New York).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described herein include compounds which otherwise correspond thereto, and which have the same general properties thereof (e.g. the ability to bind to opioid receptors), wherein one or more simple variations of substituents are made which do not adversely affect the efficacy of the compound in binding to an E2 polypeptide. In general, the compounds of the present invention may be prepared by the methods illustrated in the general reaction schemes as, for example, described below, or by modifications thereof, using readily available starting materials, reagents and conventional synthesis procedures. Thus, the contemplated equivalents include small molecule inhibitors that are capable of disrupting an interaction between a Brd4 polypeptide and an E2 polypeptide or a functional equivalent thereof. In these reactions, it is also possible to make use of variants which are in themselves known, but are not mentioned here.

Alternatively, peptides of this invention may be prepared in recombinant systems using polynucleotide sequences encoding the peptides. It is understood that a peptide of this invention may contain more than one of the above described modifications within the same peptide. Also included in this invention are pharmaceutically acceptable salt complexes of the peptides of this invention.

The invention also provides for reduction of the subject proteins to generate mimetics, e.g. peptide or non-peptide agents, which are able to mimic binding of the authentic protein to another cellular partner. Such mutagenic techniques as described below, as well as the thioredoxin system, are also particularly useful for mapping the determinants of a protein which participates in a protein-protein interaction with another protein. To illustrate, the critical residues of a protein which are involved in molecular recognition of a substrate protein may be determined and used to generate peptidomimetics that may bind to the substrate protein. The peptidomimetic may then be used as an inhibitor of the wild-type protein by binding to the substrate and covering up the critical residues needed for interaction with the wild-type protein, thereby preventing interaction of the protein and the substrate. By employing, for example, scanning mutagenesis to map the amino acid residues of a protein which are involved in binding a substrate polypeptide, peptidomimetic compounds may be generated which mimic those residues in binding to the substrate. For instance, non-hydrolyzable peptide analogs of such residues may be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), β-turn dipeptide cores (Nagai et al., (1985) Tetrahedron Lett 26:647; and Sato et al., (1986) J Chem Soc Perkin Trans 1:1231), and β-aminoalcohols (Gordon et al., (1985) Biochem Biophys Res Commun 126:419; and Dann et al., (1986) Biochem Biophys Res Commun 134:71).

A peptide mimetic is a molecule that mimics the biological activity of a peptide but is no longer peptidic in chemical nature. By strict definition, a peptidomimetic is a molecule that no longer contains any peptide bonds (that is, amide bonds between amino acids). However, the term peptide mimetic is sometimes used to describe molecules that are no longer completely peptidic in nature, such as pseudo-peptides, semi-peptides and peptoids. Examples of some peptidomimetics by the broader definition (where part of a peptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the peptide on which the peptidomimetic is based. As a result of this similar active-site geometry, the peptidomimetic has effects on biological systems which are similar to the biological activity of the peptide.

The present invention encompasses peptidomimetic compositions which are analogs that mimic the activity of biologically active peptides according to the invention, i.e., the peptidomimetics are capable of disrupting an interaction between a Brd4 polypeptide and an E2 polypeptide or a functional equivalent of an E2 polypeptide. In certain embodiments, the peptidomimetic of the invention may be substantially similar in three-dimensional shape and/or biological activity to the peptides as described herein.

Thus peptides described above have utility in the development of such small chemical compounds with similar biological activities and therefore with similar therapeutic utilities. The techniques of developing peptidomimetics are conventional. Thus, peptide bonds can be replaced by non-peptide bonds that allow the peptidomimetic to adopt a similar structure, and therefore biological activity, to the original peptide. Further modifications can also be made by replacing chemical groups of the amino acids with other chemical groups of similar structure. The development of peptidomimetics can be aided by determining the tertiary structure of the original peptide, either free or bound to a binding partner, by NMR spectroscopy, crystallography and/or computer-aided molecular modelling. These techniques aid in the development of novel compositions of higher potency and/or greater bioavailability and/or greater stability than the original peptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98, all incorporated herein by reference). Once a potential peptidomimetic compound is identified, it may be synthesized and assayed using the assays described herein to assess its activity.

Thus, through use of the methods described herein, the present invention provides compounds exhibiting enhanced therapeutic activity in comparison to the peptides described herein. The peptidomimetic compounds obtained by the above methods, having the biological activity of the above named peptides and similar three dimensional structure, are encompassed by this invention. It will be readily apparent to one skilled in the art that a peptidomimetic can be generated from any of the modified peptides described above or from a peptide bearing more than one of the modifications described above. It will furthermore be apparent that the peptidomimetics of this invention can be further used for the development of even more potent non-peptidic compounds, in addition to their utility as therapeutic compounds.

Specific examples of peptidomimetics derived from the peptides described in the previous section are presented below. These examples are illustrative and not limiting in terms of the other or additional modifications.

Peptides with a Reduced Isostere Pseudopeptide Bond [Ψ(CH₂NH)]. Proteses act on peptide bonds. It therefore follows that substitution of peptide bonds by pseudopeptide bonds confers resistance to proteolysis. A number of pseudopeptide bonds have been described that in general do not affect peptide structure and biological activity. The reduced isostere pseudopeptide bond is a suitable pseudopeptide bond that is known to enhance stability to enzymatic cleavage with no or little loss of biological activity (Couder, et al. (1993), Int. J. Peptide Protein Res., 41:181-184). Thus, the amino acid sequences of these peptides may be identical to the sequences of the L-amino acid peptides described herein except that one or more of the peptide bonds are replaced by an isostere pseudopeptide bond. Preferably the most N-terminal peptide bond is substituted, since such a substitution would confer resistance to proteolysis by exopeptidases acting on the N-terminus. The synthesis of peptides with one or more reduced isostere pseudopeptide bonds is known in the art (Couder, et al. (1993), cited above).

Peptides with a Retro-Inverso Pseudopeptide Bond [Ψ(NHCO)]. To confer resistance to proteolysis, peptide bonds may also be substituted by retro-inverso pseudopeptide bonds (Dalpozzo, et al. (1993), Int. J. Peptide Protein Res., 41:561-566, incorporated herein by reference). According to this modification, the amino acid sequences of the peptides may be identical to the sequences of the L-amino acid peptides described herein except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus. The synthesis of peptides with one or more reduced retro-inverso pseudopeptide bonds is known in the art (Dalpozzo, et al. (1993), cited above).

Peptoid Derivatives. Peptoid derivatives of peptides represent another form of modified peptides that retain the important structural determinants for biological activity, yet eliminate the peptide bonds, thereby conferring resistance to proteolysis (Simon, et al., 1992, Proc. Natl. Acad. Sci. USA, 89:9367-9371). Peptoids are oligomers of N-substituted glycines. A number of N-alkyl groups have been described, each corresponding to the side chain of a natural amino acid (Simon, et al. (1992), cited above).

In various embodiments, all or a portion of the amino acids may be replaced with the corresponding N-substituted glycine. For example, the N-terminal residue may be the only one that is replaced, or a few amino acids may be replaced by the corresponding N-substituted glycines.

Moreover, as is apparent from the present disclosure, mimetopes of the subject Brd4 peptides can be provided. Such peptidomimetics can have such attributes as being non-hydrolyzable (e.g., increased stability against proteases or other physiological conditions which degrade the corresponding peptide), increased specificity and/or potency for inhibition of PV replication, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biologyy, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). Also, see generally, Session III: Analytic and synthetic methods, in in Peptides: Chemistry and Biology, G. R. Marshall ed., ESCOM Publisher: Leiden, Netherlands, 1988)

In addition to a variety of sidechain replacements which can be carried out to generate the subject Brd4 peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

Additionally, peptidomimietics based on more substantial modifications of the backbone of the Brd4 peptide can be used. Peptidomimetics which fall in this category include (i) retro-inverso analogs, and (ii) N-alkyl glycine analogs (so-called peptoids).

Furthermore, the methods of combinatorial chemistry are being brought to bearon the development of new peptidomimetics (see e.g., Gierasch et al., Org. Lett. 5: 621-4 (2003)). For example, one embodiment of a so-called “peptide morphing” strategy focuses on the random generation of a library of peptide analogs that comprise a wide range of peptide bond substitutes.

In an exemplary embodiment, the peptidomimetic can be derived as a retro-inverso analog of the peptide.

Such retro-inverso analogs can be made according to the methods known in the art, such as that described by the Sisto et al. U.S. Pat. No. 4,522,752. For example, the illustrated retro-inverso analog can be generated as follows. The geminal diamine corresponding to the N-terminal tryptophan is synthesized by treating a protected tryptophan analog with ammonia under HOBT-DCC coupling conditions to yield the N-Boc amide, and then effecting a Hofmann-type rearrangement with I,I-bis-(trifluoroacetoxy)iodobenzene (TIB), as described in Radhakrishna et al. (1979) J. Org. Chem. 44:1746. The product amine salt is then coupled to a side-chain protected (e.g., as the benzyl ester) N-Fmoc D-lys residue under standard conditions to yield the pseudodipeptide. The Fmoc (fluorenylmethoxycarbonyl) group is removed with piperidine in dimethylformamide, and the resulting amine is trimethylsilylated with bistrimethylsilylacetamide (BSA) before condensation with suitably alkylated, side-chain protected derivative of Meldrum's acid, as described in U.S. Pat. No. 5,061,811 to Pinori et al., to yield the retro-inverso tripeptide analog WKH. The pseudotripeptide is then coupled with with an L-methionine analog under standard conditions to give the protected tetrapeptide analog. The protecting groups are removed to release the product, and the steps repeated to enlogate the tetrapeptide to the full length peptidomimetic. It will be understood that a mixed peptide, e.g. including some normal peptide linkages, will be generated. As a general guide, sites which are most susceptible to proteolysis are typically altered, with less susceptible amide linkages being optional for mimetic switching. The final product, or intermediates thereof, can be purified by HPLC.

In another illustrative embodiment, the peptidomimetic can be derived as a retro-enatio analog of the peptide. Retro-enantio analogs such as this can be synthesized commercially available D-amino acids (or analogs thereof) and standard solid- or solution-phase peptide-synthesis techniques. For example, in a preferred solid-phase synthesis method, a suitably amino-protected (t-butyloxycarbonyl, Boc) D-trp residue (or analog thereof) is covalently bound to a solid support such as chloromethyl resin. The resin is washed with dichloromethane (DCM), and the BOC protecting group removed by treatment with TFA in DCM. The resin is washed and neutralized, and the next Boc-protected D-amino acid (D-lys) is introduced by coupling with diisopropylcarbodiimide. The resin is again washed, and the cycle repeated for each of the remaining amino acids in turn (D-his, D-met, etc). When synthesis of the protected retro-enantio peptide is complete, the protecting groups are removed and the peptide cleaved from the solid support by treatment with hydrofluoric acid/anisole/dimethyl sulfide/thioanisole. The final product is purified by HPLC to yield the pure retro-enantio analog.

In still another illustrative embodiment, trans-olefin derivatives can be made for the subject polypeptide. The trans olefin analog of a Brd4 peptide can be synthesized according to the method of Y. K. Shue et al. (1987) Tetrahedron Letters 28:3225. Referring to the illustrated example, Boc-amino L-Ile is converted to the corresponding α-amino aldehyde, which is treated with a vinylcuprate to yield a diastereomeric mixture of alcohols, which are carried on together. The allylic alcohol is acetylated with acetic anhydride in pyridine, and the olefin is cleaved with osmium tetroxide/sodium periodate to yield the aldehyde, which is condensed with the Wittig reagent derived from a protected tyrosine precursor, to yield the allylic acetate. The allylic acetate is selectively hydrolyzed with sodium carbonate in methanol, and the allylic alcohol is treated with triphenylphosphine and carbon tetrabromide to yield the allylic bromide. This compound is reduced with zinc in acetic acid to give the transposed trans olefin as a mixture of diastereomers at the newly-formed center. The diastereomers are separated and the pseudodipeptide is obtained by selective transfer hydrogenolysis to unveil the free carboxylic acid. The pseudodipeptide is then coupled at the C-terminus, according to the above example, with a suitably protected tyrosine residue, and at the N-terminus with a protected alanine residue, by standard techniques, to yield the protected tetrapeptide isostere. The terapeptide is then further condensed with the olefinic tripeptide analog derived by similar means to build up the full peptide. The protecting groups are then removed with strong acid to yield the desired peptide analog, which can be further purified by HPLC.

Other pseudodipeptides can be made by the method set forth above merely by substitution of the appropriate starting Boc amino acid and Wittig reagent. Variations in the procedure may be necessary according to the nature of the reagents used, but any such variations will be purely routine and will be obvious to one of skill in the art.

It is further possible to couple the pseudodipeptides synthesized by the above method to other pseudodipeptides, to make peptide analogs with several olefinic functionalities in place of amide functionalities. For example, pseudodipeptides corresponding to Met-Arg or Tyr-Lys, etc. could be made and then coupled together by standard techniques to yield an analog of the Brd4 peptide which has alternating olefinic bonds between residues.

Still another class of peptidomimetic derivatives include the phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the the subject Brd4 peptidomimetics. To illustrate, the Brd4 peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

The subject Brd4 peptidomimetics can be optimized by, e.g., combinatorial synthesis techniques combined with such high throughput screening as described herein.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of inhibiting an interaction between a Brd4 polypeptide and an E2 protein or a functional equivalent thereof. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling. the predicted mimetope structures can then be produced by, for example, chemical synthesis, recombinant DNA technology, or by isolating a mimetope from a natural source (e.g., plants, animals, bacteria and fungi).

Nucleic Acids

One aspect of the invention pertains to isolated nucleic acids of the invention. For example, the present invention contemplates an isolated nucleic acid comprising (a) the nucleotide sequence of SEQ ID NO: 1, (b) a nucleotide sequence at least 90% identical to SEQ ID NO: 1, (c) a nucleotide sequence that hybridizes under stringent conditions to SEQ ID NO: 1, or (d) the complement of the nucleotide sequence of (a), (b) or (c). In certain embodiments, nucleic acids of the invention may be labeled, with for example, a radioactive, chemiluminescent or fluorescent label.

In another aspect, the present invention contemplates an isolated nucleic acid that selectively hybridizes under stringent conditions to at least ten nucleotides of SEQ ID NO: 1, or the complement thereof, which nucleic acid can specifically detect or amplify SEQ ID NO: 1, or the complement thereof. In yet another aspect, the present invention contemplates such an isolated nucleic acid comprising a nucleotide sequence encoding a fragment of SEQ ID NO: 2 at least 5 residues in length. The present invention further contemplates a method of hybridizing an oligonucleotide with a nucleic acid of the invention comprising: (a) providing a single-stranded oligonucleotide at least eight nucleotides in length, the oligonucleotide being complementary to a portion of a nucleic acid of the invention; and (b) contacting the oligonucleotide with a sample comprising a nucleic acid of the acid under conditions that permit hybridization of the oligonucleotide with the nucleic acid of the invention.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature.

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10× Denhardt (0.2% Ficoll, 0.2% Polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Isolated nucleic acids which differ from the nucleic acids of the invention due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (from less than 1% up to about 3 or 5% or possibly more of the nucleotides) of the nucleic acids encoding a particular protein of the invention may exist among individuals of a given species due to natural allelic variation. Any and all such nucleotide variations and resulting amino acid polymorphisms are within the scope of this invention.

Bias in codon choice within genes in a single species appears related to the level of expression of the protein encoded by that gene. Accordingly, the invention encompasses nucleic acid sequences which have been optimized for improved expression in a host cell by altering the frequency of codon usage in the nucleic acid sequence to approach the frequency of preferred codon usage of the host cell. Due to codon degeneracy, it is possible to optimize the nucleotide sequence without affecting the amino acid sequence of an encoded polypeptide. Accordingly, the instant invention relates to any nucleotide sequence that encodes all or a substantial portion of the amino acid sequence set forth in SEQ ID NO: 2 or other polypeptides of the invention.

The present invention pertains to nucleic acids encoding human Brd4 proteins and amino acid sequences evolutionarily related to a polypeptide of the invention, wherein “evolutionarily related to”, refers to proteins having different amino acid sequences which have arisen naturally (e.g. by allelic variance or by differential splicing), as well as mutational variants of the proteins of the invention which are derived, for example, by combinatorial mutagenesis.

Fragments of the polynucleotides of the invention encoding a biologically active portion of the subject polypeptides are also within the scope of the invention. Exemplary fragments are presented in the figures and the Examples. As used herein, a fragment of a nucleic acid of the invention encoding an active portion of a polypeptide of the invention refers to a nucleotide sequence having fewer nucleotides than the nucleotide sequence encoding the full length amino acid sequence of, for example, SEQ ID NO: 2, and which encodes a polypeptide which retains at least a portion of a biological activity of the full-length protein as defined herein, or alternatively, which is functional as a modulator of the biological activity of the full-length protein. For example, such fragments include a polypeptide containing a domain or short peptide fragment of the full-length protein from which the polypeptide is derived that mediates the interaction of the protein with another molecule (e.g., polypeptide, DNA, RNA, etc.). In another embodiment, the present invention contemplates an isolated nucleic acid that encodes a polypeptide having a biological activity of a human Brd4 protein. In an exemplary embodiment, the invention contemplates an isolated nucleic acid that encodes a fragment of human Brd4 that is capable of interacting with a viral E2 protein or a functional equivalent of a viral E2 protein. In another embodiment, the invention contemplates an isolated nucleic acid that encodes a fragment of human Brd4 that is capable of preventing, disrupting, and/or inhibiting an interaction between a human Brd4 protein and a viral E2 protein or a functional equivalent of a viral E2 protein.

Nucleic acids within the scope of the invention may also contain linker sequences, modified restriction endonuclease sites and other sequences useful for molecular cloning, expression or purification of such recombinant polypeptides.

A nucleic acid encoding a polypeptide of the invention may be obtained from mRNA or genomic DNA from any organism in accordance with protocols described herein, as well as those generally known to those skilled in the art. A cDNA encoding a polypeptide of the invention, for example, may be obtained by isolating total mRNA from an organism, e.g. a bacteria, virus, mammal, etc. Double stranded cDNAs may then be prepared from the total mRNA, and subsequently inserted into a suitable plasmid or bacteriophage vector using any one of a number of known techniques. A gene encoding a polypeptide of the invention may also be cloned using established polymerase chain reaction techniques in accordance with the nucleotide sequence information provided by the invention. In one aspect, the present invention contemplates a method for amplification of a nucleic acid of the invention, or a fragment thereof, comprising: (a) providing a pair of single stranded oligonucleotides, each of which is at least eight nucleotides in length, complementary to sequences of a nucleic acid of the invention, and wherein the sequences to which the oligonucleotides are complementary are at least ten nucleotides apart; and (b) contacting the oligonucleotides with a sample comprising a nucleic acid comprising the nucleic acid of the invention under conditions which permit amplification of the region located between the pair of oligonucleotides, thereby amplifying the nucleic acid.

Another aspect of the invention relates to the use of nucleic acids of the invention in “antisense therapy”. As used herein, antisense therapy refers to administration or in situ generation of oligonucleotide probes or their derivatives which specifically hybridize or otherwise bind under cellular conditions with the cellular mRNA and/or genomic DNA encoding one of the polypeptides of the invention so as to inhibit expression of that polypeptide, e.g. by inhibiting transcription and/or translation. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention may be delivered, for example, as an expression plasmid which, when transcribed in the cell, produces RNA which is complementary to at least a unique portion of the mRNA which encodes a polypeptide of the invention. Alternatively, the antisense construct may be an oligonucleotide probe which is generated ex vivo and which, when introduced into the cell causes inhibition of expression by hybridizing with the mRNA and/or genomic sequences encoding a polypeptide of the invention. Such oligonucleotide probes may be modified oligonucleotides which are resistant to endogenous nucleases, e.g. exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by van der Krol et al., (1988) Biotechniques 6:958-976; and Stein et al., (1988) Cancer Res 48:2659-2668.

In a further aspect, the invention provides double stranded small interfering RNAs (siRNAs), and methods for administering the same. siRNAs decrease or block gene expression. While not wishing to be bound by theory, it is generally thought that siRNAs inhibit gene expression by mediating sequence specific mRNA degradation. RNA interference (RNAi) is the process of sequence-specific, post-transcriptional gene silencing, particularly in animals and plants, initiated by double-stranded RNA (dsRNA) that is homologous in sequence to the silenced gene (Elbashir et al. Nature 2001; 411(6836): 494-8). Accordingly, it is understood that siRNAs and long dsRNAs having substantial sequence identity to all or a portion of SEQ ID NO: 1 may be used to inhibit the expression of a nucleic acid of the invention, and particularly when the polynucleotide is expressed in a mammalian or plant cell.

The nucleic acids of the invention may be used as diagnostic reagents to detect the presence or absence of the target DNA or RNA sequences to which they specifically bind, such as for determining the level of expression of a nucleic acid of the invention. In one aspect, the present invention contemplates a method for detecting the presence of a nucleic acid of the invention or a portion thereof in a sample, the method comprising: (a) providing an oligonucleotide at least eight nucleotides in length, the oligonucleotide being complementary to a portion of a nucleic acid of the invention; (b) contacting the oligonucleotide with a sample comprising at least one nucleic acid under conditions that permit hybridization of the oligonucleotide with a nucleic acid comprising a nucleotide sequence complementary thereto; and (c) detecting hybridization of the oligonucleotide to a nucleic acid in the sample, thereby detecting the presence of a nucleic acid of the invention or a portion thereof in the sample. In another aspect, the present invention contemplates a method for detecting the presence of a nucleic acid of the invention or a portion thereof in a sample, the method comprising: (a) providing a pair of single stranded oligonucleotides, each of which is at least eight nucleotides in length, complementary to sequences of a nucleic acid of the invention, and wherein the sequences to which the oligonucleotides are complementary are at least ten nucleotides apart; and (b) contacting the oligonucleotides with a sample comprising at least one nucleic acid under hybridization conditions; (c) amplifying the nucleotide sequence between the two oligonucleotide primers; and (d) detecting the presence of the amplified sequence, thereby detecting the presence of a nucleic acid comprising the nucleic acid of the invention or a portion thereof in the sample.

In another aspect of the invention, a nucleic acid of the invention is provided in an expression vector comprising a nucleotide sequence encoding a polypeptide of the invention and operably linked to at least one regulatory sequence. It should be understood that the design of the expression vector may depend on such factors as the choice of the host cell to be transformed and/or the type of protein desired to be expressed. The vector's copy number, the ability to control that copy number and the expression of any other protein encoded by the vector, such as antibiotic markers, should be considered.

The subject nucleic acids may be used to cause expression and over-expression of a polypeptide of the invention in cells propagated in culture, e.g. to produce proteins or polypeptides, including fusion proteins or polypeptides.

This invention pertains to a host cell transfected with a recombinant gene in order to express a polypeptide of the invention. The host cell may be any prokaryotic or eukaryotic cell. For example, a polypeptide of the present invention may be expressed in bacterial cells, such as E. coli, insect cells (baculovirus), yeast, or mammalian cells. In those instances when the host cell is human, it may or may not be in a live subject. Other suitable host cells are known to those skilled in the art. Additionally, the host cell may be supplemented with tRNA molecules not typically found in the host so as to optimize expression of the polypeptide. Other methods suitable for maximizing expression of the polypeptide will be known to those in the art.

The present invention further pertains to methods of producing the polypeptides of the invention. For example, a host cell transfected with an expression vector encoding a polypeptide of the invention may be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated.

A cell culture includes host cells, media and other byproducts. Suitable media for cell culture are well known in the art. The polypeptide may be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of a polypeptide of the invention.

Thus, a nucleotide sequence encoding all or a selected portion of polypeptide of the invention, may be used to produce a recombinant form of the protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant polypeptides of the invention by microbial means or tissue-culture technology in accord with the subject invention.

Expression vehicles for production of a recombinant protein include plasmids and other vectors. For instance, suitable vectors for the expression of a polypeptide of the invention include plasmids of the types: pBR322-derived plasmids, pEMBL-derived plasmids, pEX-derived plasmids, pBTac-derived plasmids and pUC-derived plasmids for expression in prokaryotic cells, such as E. coli. A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83). In certain embodiments, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells.

When expression of a carboxy terminal fragment of a polypeptide is desired, i.e. a truncation mutant, it may be necessary to add a start codon (ATG) to the oligonucleotide fragment containing the desired sequence to be expressed. It is well known in the art that a methionine at the N-terminal position may be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, may be achieved either in vivo by expressing such recombinant polypeptides in a host which produces MAP (e.g., E. coli or CM89 or S. cerevisiae), or in vitro by use of purified MAP (e.g., procedure of Miller et al.).

Coding sequences for a polypeptide of interest may be incorporated as a part of a fusion gene including a nucleotide sequence encoding a different polypeptide. The present invention contemplates an isolated nucleic acid comprising a nucleic acid of the invention and at least one heterologous sequence encoding a heterologous peptide linked in frame to the nucleotide sequence of the nucleic acid of the invention so as to encode a fusion protein comprising the heterologous polypeptide. The heterologous polypeptide may be fused to (a) the C-terminus of the polypeptide encoded by the nucleic acid of the invention, (b) the N-terminus of the polypeptide, or (c) the C-terminus and the N-terminus of the polypeptide. In certain instances, the heterologous sequence encodes a polypeptide permitting the detection, isolation, solubilization and/or stabilization of the polypeptide to which it is fused. In still other embodiments, the heterologous sequence encodes a polypeptide selected from the group consisting of a polyHis tag, myc, HA, GST, protein A, protein G, calmodulin-binding peptide, thioredoxin, maltose-binding protein, poly arginine, poly His-Asp, FLAG, a portion of an immunoglobulin protein, and a transcytosis peptide.

Fusion expression systems can be useful when it is desirable to produce an immunogenic fragment of a polypeptide of the invention. For example, the VP6 capsid protein of rotavirus may be used as an immunologic carrier protein for portions of polypeptide, either in the monomeric form or in the form of a viral particle. The nucleic acid sequences corresponding to the portion of a polypeptide of the invention to which antibodies are to be raised may be incorporated into a fusion gene construct which includes coding sequences for a late vaccinia virus structural protein to produce a set of recombinant viruses expressing fusion proteins comprising a portion of the protein as part of the virion. The Hepatitis B surface antigen may also be utilized in this role as well. Similarly, chimeric constructs coding for fusion proteins containing a portion of a polypeptide of the invention and the poliovirus capsid protein may be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

Fusion proteins may facilitate the expression and/or purification of proteins. For example, a polypeptide of the present invention may be generated as a glutathione-S-transferase (GST) fusion protein. Such GST fusion proteins may be used to simplify purification of a polypeptide of the invention, such as through the use of glutathione-derivatized matrices (see, for example, Current Protocols in Molecular Biology, eds. Ausubel et al., (N.Y.: John Wiley & Sons, 1991)). In another embodiment, a fusion gene coding for a purification leader sequence, such as a poly-(His)/enterokinase cleavage site sequence at the N-terminus of the desired portion of the recombinant protein, may allow purification of the expressed fusion protein by affinity chromatography using a Ni²⁺ metal resin. The purification leader sequence may then be subsequently removed by treatment with enterokinase to provide the purified protein (e.g., see Hochuli et al., (1987) J. Chromatography 411: 177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992).

The present invention further contemplates a transgenic non-human animal having cells which harbor a transgene comprising a nucleic acid of the invention.

In other embodiments, the invention provides for nucleic acids of the invention immobilized onto a solid surface, including, plates, microtiter plates, slides, beads, particles, spheres, films, strands, precipitates, gels, sheets, tubing, containers, capillaries, pads, slices, etc. The nucleic acids of the invention may be immobilized onto a chip as part of an array. The array may comprise one or more polynucleotides of the invention as described herein. In one embodiment, the chip comprises one or more polynucleotides of the invention as part of an array of mammalian polynucleotide sequences.

In still other embodiments, the invention comprises the sequence of a nucleic acid of the invention in computer readable format. The invention also encompasses a database comprising the sequence of a nucleic acid of the invention.

Antibodies

Another aspect of the invention pertains to antibodies specifically reactive with a polypeptide of the invention. For example, by using peptides based on a polypeptide of the invention, e.g., having an amino acid sequence of SEQ ID NO: 2 or an immunogenic fragment thereof, antisera or monoclonal antibodies may be made using standard methods. An exemplary immunogenic fragment may contain five, eight, ten or more consecutive amino acid residues of SEQ ID NO: 2.

An antibody may be monoclonal or polyclonal. The antibody may be a member of any immunoglobulin class, including any of the human classes: IgG, IgM, IgA, IgD, and IgE. The antibodies may be bispecific or chimeric molecules, as well as trimeric antibodies, humanized antibodies, human antibodies, and single chain antibodies. All of these modified forms of antibodies as well as fragments of antibodies are intended to be included in the term “antibody”.

In other embodiments, the invention provides antibodies that bind to complexes containing Brd4, such as complexes comprising Brd4 and an E2 protein or a functional equivalent of an E2 protein. In one embodiment, the present invention provides an isolated antibody that has a higher binding affinity for a Brd4/E2 complex than for the individual complex polypeptides. In another embodiment, the present invention provides an isolated antibody that binds to an interaction site on Brd4, E2 or a functional equivalent of an E2 protein. In still other embodiments, the isolated antibodies of the invention disrupt or stabilize a Brd4/E2 complex. In yet another embodiment, the present invention provides an isolated antibody that binds to a Brd4 polypeptide comprising the amino acid sequence of residues 1047-1362, 1134-1362, and/or 1224-1362 of Brd4. Another aspect of the invention pertains to antibodies specifically reactive with a Brd4 polypeptide.

Antibody fragments may also be used. Examples of antibody fragments include, but are not limited to, Fab, Fab′, F(ab′)₂, single chain (scFv), scFv, Fv, dsFv diabody, and Fd fragments.

In one aspect, the present invention contemplates a purified antibody that binds specifically to a polypeptide of the invention and which does not substantially cross-react with a protein which is less than about 80%, or less than about 90%, identical to a polypeptide of the invention. In another aspect, the present invention contemplates an array comprising a substrate having a plurality of address, wherein at least one of the addresses has disposed thereon a purified antibody that binds specifically to a polypeptide of the invention.

Antibodies directed against the polypeptides of the invention can be used to isolate a polypeptide of the invention by standard techniques, such as affinity chromatography or immunoprecipitation. The antibodies may also be used to facilitate the purification of a Brd4 polypeptide from cells obtained from a patient sample or from a cell culture. In addition, such antibodies are useful to detect the presence of a polypeptide of the invention in cells or tissues to determine the pattern of expression of the polypeptide among various tissues in an organism and/or over the course of normal development. Further, such antibodies can be used to detect protein in situ, in vitro, or in a cell lysate or supernatant, in order to evaluate the abundance and pattern of expression. Also, such antibodies can be used to assess abnormal tissue distribution or abnormal expression during development or progression of a biological condition.

Further, the antibodies directed against the polypeptides of the invention can be used to assess expression in disease states, including active stages of the disease or pre-disease states to asses an individual's predisposition toward a disease or disorder. When a disorder is caused by an inappropriate tissue distribution, developmental expression, level of expression of the protein, or expressed/processed form, the antibody can be prepared against the normal protein. If a disorder is characterized by a specific mutation in the protein, antibodies specific for this mutant protein can be used to assay for the presence of the specific mutant protein.

The antibodies directed against the polypeptides of the invention can also be used to assess subcellular localization of Brd4 in the various tissues in an organism. The diagnostic uses can be applied, not only in diagnostic applications, but also in monitoring a treatment modality. Accordingly, where a treatment is ultimately aimed at correcting expression level, aberrant tissue distribution, or developmental expression, antibodies directed against the protein or relevant fragments can be used to monitor therapeutic efficacy. For example, antibodies may be used to monitor the effect of modulators of Brd4 complexes, e.g., when administered to a subject. In particular, antibodies to Brd4 complexes can be used to monitor the level of Brd4 complexes.

Additionally, antibodies directed against the polypeptides of the invention are useful in pharmacogenomic analysis. For example, antibodies prepared against polymorphic proteins can be used to identify individuals that require modified treatment modalities. The antibodies are also useful as diagnostic tools, for example, as an immunological marker for aberrant protein which may analyzed by a variety of techniques, including, electrophoretic mobility, isoelectric point, proteolytic digest, and other assays known to those in the art.

Antibodies directed against the polypeptides of the invention can be used to selectively block the action of the polypeptides of the invention. Antibodies against a polypeptide of the invention may be employed to treat diseases or disorders related to a viral infection. For example, the present invention contemplates a method for treating a subject suffering from a disease or disorder related to a viral infection, comprising administering to an animal having the condition a therapeutically effective amount of a purified antibody that binds specifically to a polypeptide of the invention.

The invention also encompasses kits comprising antibodies directed against the polypeptides of the invention for use in detecting the presence of a protein in a biological sample. The kit may comprise one or more of the following: antibodies, such as a labeled or labelable antibody; a compound or agent for detecting protein in a biological sample; means for determining the amount of protein in the sample; means for comparing the amount of protein in the sample with a standard; and instructions for use. Such a kit can be supplied to detect a single protein or epitope or can be configured to detect one of a multitude of epitopes, such as in an antibody detection array.

In other embodiments, the antibodies of the invention, or variants thereof, are modified to make them less immunogenic when administered to a subject. For example, if the subject is human, the antibody may be “humanized”; where the complimentarity determining region(s) of the hybridoma-derived antibody has been transplanted into a human monoclonal antibody, for example as described in Jones, P. et al. (1986), Nature 321, 522-525 or Tempest et al. (1991) Biotechnology 9, 266-273. Also, transgenic mice, or other mammals, may be used to express humanized antibodies. Such humanization may be partial or complete.

The use of a nucleic acid of the invention in genetic immunization may employ a suitable delivery method such as direct injection of plasmid DNA into muscles (Wolff et al., Hum Mol Genet 1992, 1:363, Manthorpe et al., Hum. Gene Ther. 1963:4, 419), delivery of DNA complexed with specific protein carriers (Wu et al., J Biol. Chem. 1989: 264, 16985), coprecipitation of DNA with calcium phosphate (Benvenisty & Reshef, PNAS USA, 1986:83, 9551), encapsulation of DNA in various forms of liposomes (Kaneda et al., Science 1989:243, 375), particle bombardment (Tang et al., Nature 1992, 356:152, Eisenbraun et al., DNA Cell Biol 1993, 12:791) and in vivo infection using cloned retroviral vectors (Seeger et al., PNAS USA 1984:81, 5849).

Methods of Producing, Identifying, and Isolating Brd4 Complexes

In another aspect the invention provides methods of producing, identifying, and isolating a Brd4 complex. Brd4 complexes may be produced by a variety of methods. For example, Brd4 complexes may be naturally-occurring, for instance in a cell infected with a virus (such as, for example, a papillomavirus, herpes virus, epstein barr virus, etc.), or produced in a host cell comprising nucleic acids encoding Brd4 and/or E2 or a functional equivalent thereof, or produced in vitro in a solution comprising Brd4 polypeptides and at an E2 protein or functional equivalent thereof.

The term “Brd4 complex polypeptide” refers to an individual polypeptide that may be present in a Brd4 complex, including Brd4 and polypeptides that may interact with Brd4 either directly or indirectly. In exemplary embodiments, a Brd4 complex polypeptide refers to Brd4, E2, and functional equivalents of E2. In other embodiments, a Brd4 complex polypeptide refers to a fusion protein comprising all or a portion of one or more Brd4 complex polypeptides such as Brd4 and/or E2 (or a functional equivalent thereof).

The term “complex”, as applied to two moieties, refers to an association between at least two moieties (e.g. chemical or biochemical) that have an affinity for one another. Examples of complexes include associations between antigen/antibodies, lectin/avidin, target polynucleotide/probe oligonucleotide, antibody/anti-antibody, receptor/ligand, enzyme/ligand and the like. “Member of a complex” refers to one moiety of the complex, such as an antigen or ligand. “Protein complex” or “polypeptide complex” refers to a complex comprising at least one polypeptide. In certain exemplary embodiments, a complex refers to a “Bdr4 complex” comprising Brd4 and at least one other molecule. In exemplary embodiments, a Brd4 complex comprises (a) Brd4 and an E2 polypeptide or a functional equivalent of an E2 polypeptide; (b) Brd4 and a fragment of an E2 polypeptide or a functional equivalent of an E2 polypeptide; (c) a fragment of Brd4 and an E2 polypeptide or a functional equivalent of an E2 polypeptide; or (d) a fragment of Brd4 and a fragment of an E2 polypeptide or a functional equivalent of an E2 polypeptide. In one embodiment, the complex comprises (a) an amino acid sequence set forth in SEQ ID NO: 2; (b) an amino acid sequence having at least 95% identity with the amino acid sequence set forth in SEQ ID NO: 2; or (c) an amino acid sequence encoded by a polynucleotide that hybridizes under stringent conditions to the complementary strand of a polynucleotide having SEQ ID NO: 1 and wherein said polypeptide has at least one biological activity of a Brd4 protein. In another embodiment, the complex comprises a fragment of Brd4 having residues 1047-1362, 1134-1362, or 1224-1362 of SEQ ID NO: 2. In another embodiment, the complex comprises a fragment having at least five consecutive amino acid residues from SEQ ID NO: 2 or a region of SEQ ID NO: 2 having amino acid residues 1047-1362, 1134-1362, or 1224-1362 of SEQ ID NO: 2.

The term “binding” or “interacting”, as applied to two molecules, refers to an association, which may be a stable association, between the two molecules, e.g., between a polypeptide of the invention and a binding partner, due to, for example, electrostatic, hydrophobic, ionic and/or hydrogen-bond interactions under physiological conditions.

The term “E2 polypeptide” is known in the art and refers to a viral protein that participates in viral replication, viral transcription, and/or regulation of cellular transformation. In an exemplary embodiment, an E2 protein is capable of interacting with a Brd4 protein. E2 polypeptides typically are composed of two well-conserved functional domains. The E2 carboxy-terminus generally includes a DNA binding domain that binds as a dimer to the ACCN₆GGT recognition sequence (Andropy et al., Nature, 1987, 325, 70). The E2 amino-terminus typically features a transcriptional activation domain that regulates viral gene expression and interacts with components of the host cell apparatus. The E2 amino-terminus also interacts with the E1 protein. These amino-terminal and the carboxy-terminal domains are connected by a hinge region that is dispensable for both replication and transcriptional activation. In exemplary embodiments, E2 proteins in accordance with the invention include, for example, E2 proteins from papillomaviruses, Epstein Barr viruses, and Herpes viruses. Exemplary E2 proteins include, for example, the E2 proteins from bovine papilloma virus 1 (BPV1), human papilloma viruses (HPV) 16, 6b, 11, 18, 31, 1A, and 57 (see e.g., Sakai et al., J. Virology 70: 1602-1611 (1996) for sequences of a variety of exemplary E2 proteins).

The term “functional equivalent of an E2 polypeptide” refers to a viral protein that may share little sequence identity (e.g., less than 80%, 70%, 50%, 40%, 30%, 20%, 10%, or less) or structural similarity to an E2 protein but carries out at least one biological activity similar to that of an E2 protein. For example, a functional equivalent of an E2 protein may participate in viral replication, viral transcription, and/or regulation of cellular transformation. In an exemplary embodiment, a functional equivalent of an E2 protein is capable of interacting with a Brd4 protein. An example of a functional equivalent of an E2 polypeptide is the latency-associated nuclear antigen (LANA) protein from Kaposi sarcoma-associated herpesvirus (KSHV).

A variety of materials may be used as the source of potential Brd4 and/or E2 polypeptides (or functional equivalents thereof). In one embodiment, a cellular extract or extracellular fluid may be used. The choice of starting material for the extract may be based upon the cell or tissue type or type of fluid that would be expected to contain Brd4 complex polypeptides. Micro-organisms or other organisms are grown in a medium that is appropriate for that organism and can be grown in specific conditions to promote the expression of proteins that may interact with the target protein. Exemplary starting material that may be used to make a suitable extract are: 1) one or more types of tissue derived from an animal, especially a human, 2) cells grown in tissue culture that were derived from an animal, especially a human, 3) micro-organisms grown in suspension or non-suspension cultures, 4) virus-infected cells, 5) purified organelles (including, but not restricted to nuclei, mitochondria, membranes, Golgi, endoplasmic reticulum, lysosomes, or peroxisomes) prepared by differential centrifugation or another procedure from animal, especially human, cells, 6) serum or other bodily fluids including, but not limited to, blood, urine, semen, synovial fluid, cerebrospinal fluid, amniotic fluid, lymphatic fluid or interstitial fluid. In other embodiments, a total cell extract may not be the optimal source of Brd4 complex polypeptides.

In an alternative embodiment, a Brd4 complex polypeptide (e.g., Brd4, E2, or a functional equivalent of an E2 polypeptide) is expressed, optionally in a heterologous cell, and purified and then mixed with a potential Brd4 complex polypeptide or mixture of polypeptides to identify Brd4 complex formation. The potential Brd4 complex polypeptide may be a single purified or semi-purified protein, or a mixture of proteins, including a mixture of purified or semi-purified proteins, a cell lysate, a clarified cell lysate, a semi-purified cell lysate, etc.

Typically, it will be desirable to immobilize a Brd4 complex polypeptide or Brd4 complex to facilitate separation of Brd4 complexes from uncomplexed forms of the interacting proteins, as well as to accommodate automation of the assay. The Brd4 complex or Brd4 complex polypeptide, or ligand, may be immobilized onto a solid support (e.g., column matrix, microtiter plate, slide, etc.). In certain embodiments, the ligand may be purified. In certain instances, a fusion protein may be provided which adds a domain that permits the ligand to be bound to a support.

In various in vitro embodiments, the set of proteins engaged in a protein-protein interaction comprises a cell extract, a clarified cell extract, or a reconstituted protein mixture of at least semi-purified proteins. By semi-purified, it is meant that the proteins utilized in the reconstituted mixture have been previously separated from other cellular or viral proteins. For instance, in contrast to cell lysates, the proteins involved in a protein-protein interaction are present in the mixture to at least about 50% purity relative to all other proteins in the mixture, and more preferably are present in greater, even 90-95%, purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure activity resulting from the given protein-protein interaction.

The present invention contemplates a method for identifying a Brd4 complex or Brd4 complex polypeptide, the method comprising: (a) exposing a sample to a solid substrate coupled to a Brd4 complex or Brd4 complex polypeptide under conditions which promote protein-protein interactions; (b) washing the solid substrate so as to remove any polypeptides interacting non-specifically with the polypeptide or fragment; (c) eluting the polypeptides which specifically interact with the Brd4 complex or Brd4 complex polypeptide; and (d) identifying the interacting protein. The interacting protein may be identified by a number of methods, including mass spectrometry, gel electrophoresis, activity assay, or protein sequencing.

In another aspect, the present invention contemplates a method for identifying a protein capable of interacting with Brd4, a Brd4 complex polypeptide, or Brd4 complex, or fragments thereof, the method comprising: (a) subjecting a sample to protein-affinity chromatography on multiple columns, the columns having a Brd4 complex or Brd4 complex polypeptide coupled to the column matrix in varying concentrations, and eluting bound components of the extract from the columns; (b) separating the components to isolate a polypeptide capable of interacting with the Brd4 polypeptide, complex or fragment; and (c) analyzing the interacting protein by mass spectrometry to identify the interacting protein. In certain instances, the foregoing method will use polyacrylamide gel electrophoresis to separate and/or analyze the interacting polypeptides.

In another aspect, the present invention contemplates a method for identifying a Brd4 complex or Brd4 complex polypeptide the method comprising: (a) subjecting a cellular extract or extracellular fluid to protein-affinity chromatography on multiple columns, the columns having a Brd4 complex or Brd4 complex polypeptide coupled to the column matrix in varying concentrations, and eluting bound components of the extract from the columns; (b) gel-separating the components to isolate an interacting protein; wherein the interacting protein is observed to vary in amount in direct relation to the concentration of coupled polypeptide or fragment; (c) digesting the interacting protein to give corresponding peptides; (d) analyzing the peptides by MALDI-TOF mass spectrometry or post source decay to determine the peptide masses; and (e) performing correlative database searches with the peptide, or peptide fragment, masses, whereby the interacting protein is identified based on the masses of the peptides or peptide fragments. The foregoing method may include the further step of including the identifies of any interacting proteins into a relational database.

In another embodiment, proteins that interact with a Brd4 complex or Brd4 complex polypeptide may be identified using affinity chromatography. In one aspect, for affinity chromatography using a solid support, a Brd4 complex polypeptide may be attached by a variety of means known to those of skill in the art. For example, the polypeptide may be coupled directly (through a covalent linkage) to commercially available pre-activated resins as described in Formosa et al., Methods in Enzymology 1991, 208, 24-45; Sopta et al, J. Biol. Chem. 1985, 260, 10353-60; Archambault et al., Proc. Natl. Acad. Sci. USA 1997, 94, 14300-5. Alternatively, the polypeptide may be tethered to the solid support through high affinity binding interactions. If the polypeptide is expressed fused to a tag, such as GST, the fusion tag can be used to anchor the polypeptide to the matrix support, for example Sepharose beads containing immobilized glutathione. Solid supports that take advantage of these tags are commercially available.

In other embodiments, Brd4 complexes may be isolated using immunoprecipitation. The cells expressing a Brd4 complex polypeptide are lysed under conditions which maintain protein-protein interactions, and Brd4 complexes are isolated. In certain embodiments, it may be desirable to use a tagged version of a Brd4 complex polypeptide in order to facilitate isolation of complexes from the reaction mixture. Suitable tags for immunoprecipitation experiments include HA, myc, FLAG, HIS, GST, protein A, protein G, etc. Immunoprecipitation from a cell lysate or other protein mixture may be carried out using an antibody specific for a Brd4 complex or Brd4 complex polypeptide or using an antibody which recognizes a tag to which a Brd4 complex polypeptide is fused (e.g., anti-HA, anti-myc, anti-FLAG, etc.). Antibodies specific for a variety of tags are known to the skilled artisan and are commercially available from a number of sources. In the case where an complex polypeptide is fused to a His, GST, or protein A/G tag, immunoprecipitation may be carried out using the appropriate affinity resin (e.g., beads functionalized with Ni, glutathione, Fc region of IgG, etc.). Test compounds which modulate a protein-protein interaction involving a Brd4 complex polypeptide may be identified by carrying out the immunoprecipitation reaction in the presence and absence of the test agent and comparing the level and/or activity of the Brd4 complex between the two reactions.

Complex formation between a Brd4 complex polypeptide and a binding partner may be detected by a variety of methods. Modulation of the formation of Brd4 complexes may be quantitated using, for example, detectably labeled proteins such as radiolabeled, fluorescently labeled, or enzymatically labeled polypeptides or binding partners, by immunoassay, or by chromatographic detection. Methods of isolating and identifying Brd4 complexes described in above may be incorporated into the detection methods.

Typically, it will be desirable to immobilize a Brd4 complex polypeptide or its binding partner to facilitate separation of Brd4 complexes from uncomplexed forms of one or both of the proteins, as well as to accommodate automation of the assay. Binding of a Brd4 complex polypeptide to a binding partner may be accomplished in any vessel suitable for containing the reactants. Examples include microtitre plates, test tubes, and micro-centrifuge tubes. In one embodiment, a fusion protein may be provided which adds a domain that allows the protein to be bound to a matrix. For example, glutathione-S-transferase/polypeptide (GST/polypeptide) fusion proteins may be adsorbed onto glutathione sepharose beads (Sigma Chemical, St. Louis, Mo.) or glutathione derivatized microtitre plates, which are then combined with the binding partner, e.g. an ³⁵S-labeled binding partner, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintillant), or in the supernatant after the complexes are subsequently dissociated. Alternatively, the complexes may be dissociated from the matrix, separated by SDS-PAGE, and the level of Brd4 complex polypeptide or binding partner found in the bead fraction quantitated from the gel using standard electrophoretic techniques such as described in the appended examples.

For processes that rely on immunodetection for quantitating one of the Brd4 complex polypeptides trapped in the Brd4 complex, antibodies against the Brd4 complex polypeptide, such as anti-Brd4 or anti-E2 antibodies, may be used. Alternatively, the Brd4 complex polypeptide to be detected in the Brd4 complex may be “epitope-tagged” in the form of a fusion protein that includes, in addition to the polypeptide sequence, a second polypeptide for which antibodies are readily available (e.g. from commercial sources). For instance, the GST fusion proteins described above may also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, NJ).

In certain in vitro embodiments of the present assay, the protein or the set of proteins engaged in a protein-protein, protein-substrate, or protein-nucleic acid interaction comprises a reconstituted protein mixture of at least semi-purified proteins. By semi-purified, it is meant that the proteins utilized in the reconstituted mixture have been previously separated from other cellular or viral proteins. For instance, in contrast to cell lysates, the proteins involved in a protein-substrate, protein-protein or nucleic acid-protein interaction are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure activity resulting from the given protein-substrate, protein-protein interaction, or nucleic acid-protein interaction.

In one embodiment, the use of reconstituted protein mixtures allows more careful control of the protein-substrate, protein-protein, or nucleic acid-protein interaction conditions. Moreover, the system may be derived to favor discovery of modulators of particular intermediate states of the protein-protein interaction. For instance, a reconstituted protein assay may be carried out both in the presence and absence of a candidate agent, thereby allowing detection of a modulator of a given protein-substrate, protein-protein, or nucleic acid-protein interaction.

Assaying biological activity resulting from a given protein-substrate, protein-protein or nucleic acid-protein interaction, in the presence and absence of a candidate modulator, may be accomplished in any vessel suitable for containing the reactants. Examples include microtitre plates, test tubes, and micro-centrifuge tubes.

In still further embodiments, the Brd4 complex of interest is generated in whole cells, taking advantage of cell culture techniques to support the subject assay. For example, the Brd4 complex of can be constituted in a prokaryotic or eukaryotic cell culture system. Advantages to generating the Brd4 complex in an intact cell includes the ability to screen for modulators of the level or activity of the Brd4 complex which are functional in an environment more closely approximating that which therapeutic use of the modulator would require, including the ability of the agent to gain entry into the cell. Furthermore, certain of the in vivo embodiments of the assay are amenable to high through-put analysis of candidate agents.

The Brd4 complexes and Brd4 complex polypeptides can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein. Moreover, in the whole cell embodiments of the subject assay, the reporter gene construct can provide, upon expression, a selectable marker. Such embodiments of the subject assay are particularly amenable to high through-put analysis in that proliferation of the cell can provide a simple measure of the protein-protein interaction.

The amount of transcription from the reporter gene may be measured using any method known to those of skill in the art to be suitable. For example, specific mRNA expression may be detected using Northern blots or specific protein product may be identified by a characteristic stain, western blots or an intrinsic activity. In certain embodiments, the product of the reporter gene is detected by an intrinsic activity associated with that product. For instance, the reporter gene may encode a gene product that, by enzymatic activity, gives rise to a detection signal based on color, fluorescence, or luminescence.

Identification of Compounds that Modulate Brd4 Complexes

Modulators of Brd4 complexes and Brd4 complex polypeptides, may be identified and developed as set forth below and otherwise using techniques and methods known to those of skill in the art. The modulators of the invention may be employed, for instance, to inhibit and treat virus-mediated diseases or disorders. The modulators of the invention may also serve as modulators of virus-mediated diseases or disorders via action on a Brd4 complex polypeptide. The modulators of the invention may elicit a change in any of the activities selected from the group consisting of (a) a change in the level of a Brd4 complex, (b) a change in the activity of a Brd4 complex, (c) a change in the stability of a Brd4 complex, (d) a change in the conformation of a Brd4 complex, (e) a change in the activity of at least one polypeptide contained within a Brd4 complex, (f) a change in the conformation of at least one polypeptide contained within a Brd4 complex, (g) where the reaction mixture is a whole cell, a change in the intracellular localization of a Brd4 complex or a Brd4 complex polypeptide thereof, (h) where the reaction mixture is a whole cell, a change the transcription level of a gene dependent on a Brd4 complex, and (i) where the reaction mixture is a whole cell, a change in second messenger levels in the cell. A number of methods for identifying a molecule which modulates a Brd4 complex or a Brd4 complex polypeptide are known in the art. For example, in one such method, a Brd4 complex or a Brd4 complex polypeptide is contacted with a test compound, and the activity of the Brd4 complex or Brd4 complex polypeptide in the presence of the test compound is determined, wherein a change in the activity of the Brd4 complex or Brd4 complex polypeptide is indicative that the test compound modulates the activity of Brd4 complex or Brd4 complex polypeptide.

Compounds to be tested for their ability to act as modulators of Brd4 complexes or Brd4 complex polypeptides can be produced, for example, by bacteria, yeast or other organisms (e.g. natural products), produced chemically (e.g. small molecules, including peptidomimetics), or produced recombinantly. Compounds for use with the above-described methods may be selected from the group of compounds consisting of lipids, carbohydrates, polypeptides, peptidomimetics, peptide-nucleic acids (PNAs), small molecules, natural products, aptamers and polynucleotides. In certain embodiments, the compound is a polynucleotide. In some embodiments, said polynucleotide is an antisense nucleic acid. In other embodiments, said polynucleotide is an siRNA. In certain embodiments, the compound comprises a Brd4 complex polypeptide or polynucleotide encoding a Brd4 complex polypeptide as described above. In certain embodiments, the compound may be a member of a library of compounds.

A variety of assay formats will suffice and, in light of the present disclosure, those not expressly described herein will nevertheless be comprehended by one of ordinary skill in the art. Assay formats for Brd4 complex formation or enzymatic activity of a Brd4 complex complex or Brd4 complex polypeptides can be generated in many different forms, and include assays based on cell-free systems, e.g. purified proteins or cell lysates, as well as cell-based assays which utilize intact cells. Simple binding assays can also be used to detect agents which, by disrupting the formation of Brd4 complexes, or the binding of a Brd4 complex or Brd4 complex polypeptide to a substrate, can serve as a modulator. Another example of an assay for a modulator of a Brd4 complex polypeptide is a competitive assay that combines a Brd4 complex polypeptide and a potential modulator with Brd4 complex polypeptides, recombinant molecules that comprise a Brd4 complex, Brd4 complex, natural substrates or ligands, or substrate or ligand mimetics, under appropriate conditions for a competitive inhibition assay. Brd4 complex polypeptides can be labeled, such as by radioactivity or a colorimetric compound, such that the number of molecules of a Brd4 complex polypeptide bound to a binding molecule or converted to product can be determined accurately to assess the effectiveness of the potential modulator.

Assays may employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof. Assays may also employ any of the methods for isolating, preparing and detecting Brd4 complexes as described above.

In many drug screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target. Accordingly, potential modifiers, e.g., modulators of Brd4 complexes may be detected in a cell-free assay generated by constitution of a functional Brd4 complex in a cell lysate. In an alternate format, the assay can be derived as a reconstituted protein mixture which, as described below, offers a number of benefits over lysate-based assays.

In certain embodiments, methods for identifying a compound that modulates an virus mediated disease or disorder are provided, comprising: (i) contacting a Brd4 complex with a test compound; and (ii) assessing the extent of said virus mediated disease or disorder, wherein a modulation in the extent of said virus mediated disease or disorder in the presence of said test compound indicates that the test compound may be a candidate therapeutic for said virus mediated disease or disorder. For example, the extent of a virus mediated cancer could be evaluated by medical diagnostic techniques known to one of skill in the art, such as, for example, biopsy, early antigen serum titer, serum lactate dehydrogenase levels, immunophenotyping, and the like.

In another embodiment, the activity of a Brd4 complex may be determined by examining the level of Brd4 complex that is formed or present in a sample.

In another embodiment, the activity of a Brd4 complex or Brd4 complex polypeptide may be determined by assaying for the level of expression of RNA and/or protein molecules. Transcription levels may be determined, for example, using Northern blots, hybridization to an oligonucleotide array or by assaying for the level of a resulting protein product. Translation levels may be determined, for example, using Western blotting or by identifying a detectable signal produced by a protein product (e.g., fluorescence, luminescence, enzymatic activity, etc.). Depending on the particular situation, it may be desirable to detect the level of transcription and/or translation of a single gene or of multiple genes.

In other embodiments, the biological activity of a Brd4 complex or Brd4 complex polypeptide can be assessed by monitoring changes in the phenotype of the targeted cell. For example, the detection means can include a reporter gene construct which includes a transcriptional regulatory element that is dependent in some form on the level of a Brd4 complex or Brd4 complex polypeptide. The Brd4 complex can be provided as a fusion protein with a domain that binds to a DNA element of the reporter gene construct. The added domain of the fusion protein can be one which, through its DNA-binding ability, increases or decreases transcription of the reporter gene. Which ever the case may be, its presence in the fusion protein renders it responsive to a Brd4 complex or Brd4 complex polypeptide. Accordingly, the level of expression of the reporter gene will vary with the level of expression of a Brd4 complex or Brd4 complex polypeptide.

Moreover, in the whole cell embodiments of the subject assay, the reporter gene construct can provide, upon expression, a selectable marker. A reporter gene includes any gene that expresses a detectable gene product, which may be RNA or protein. Preferred reporter genes are those that are readily detectable. The reporter gene may also be included in the construct in the form of a fusion gene with a gene that includes desired transcriptional regulatory sequences or exhibits other desirable properties. For instance, the product of the reporter gene can be an enzyme which confers resistance to antibiotic or other drug, or an enzyme which complements a deficiency in the host cell (i.e. thymidine kinase or dihydrofolate reductase). To illustrate, the aminoglycoside phosphotransferase encoded by the bacterial transposon gene Tn5 neo can be placed under transcriptional control of a promoter element responsive to the level of a Brd4 complex or Brd4 complex polypeptide present in the cell. Such embodiments of the subject assay are particularly amenable to high through-put analysis in that proliferation of the cell can provide a simple measure of inhibition of the Brd4 complex or Brd4 complex polypeptide.

Exemplary Uses

The methods and compositions described herein may be used for the treatment or prevention of diseases or disorders associated with a variety of viral infections. The methods and compositions described herein may be used to treat or prevent viral infections (or diseases or disorders associated therewith) in any type of organism that is subject to infection by a virus, including, for example, animals (e.g., mammals, birds, rodents, amphibians, etc.), plants, and bacteria. Accordingly, the methods and compositions of the invention have utility in wide ranging fields such as, for example, agriculture, livestock, crops, medical treatments, combating bio-terrorism, etc.

Examples of disease causing viruses that may be treated in accord with the compositions and methods described herein include: Papovaviridae (papilloma viruses, polyoma viruses); Herpesviridae (herpes simplex virus (HSV) 1 and 2, varicella zoster virus, cytomegalovirus (CMV), herpes viruses'); Retroviridae (e.g., human immunodeficiency viruses, such as HIV-1 (also referred to as HTLV-III, LAV or HTLV-III/LAV, See Ratner, L. et al., Nature, Vol. 313, Pp. 227-284 (1985); Wain Hobson, S. et al, Cell, Vol. 40: Pp. 9-17 (1985)); HIV-2 (See Guyader et al., Nature, Vol. 328, Pp. 662-669 (1987); European Patent Publication No. 0 269 520; Chakraborti et al., Nature, Vol. 328, Pp. 543-547 (1987); and European Patent Application No. 0 655 501); and other isolates, such as HIV-LP (International Publication No. WO 94/00562 entitled “A Novel Human Immunodeficiency Virus”); Picornaviridae (e.g., polio viruses, hepatitis A virus, (Gust, I. D., et al., Intervirology, Vol. 20, Pp. 1-7 (1983); entero viruses, human coxsackie viruses, rhinoviruses, echoviruses);Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reovi ruses, orbiviurses and rotavi ruses); Birnaviridae; Hepadnaviridae (Hepatitis B virus); Parvoviridae (parvoviruses); Adenoviridae (most adenoviruses); Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Genomic information for over 900 viral species is available from TIGR and/or NCBI, including, for example, information about deltaviruses, retroid viruses, satellites, dsDNA viruses, dsRNA viruses, ssDNA viruses, ssRNA negative-strand viruses, ssRNA positive-strand viruses, unclassified bacteriophages, and other unclassified viruses.

In another embodiment, the methods and compositions described herein may be used for combating viral based biological warfare agents. Examples of viral based biological warfare agents, include, for example, filoviruses (e.g., ebola or Marburg), arenaviruses (e.g., Lassa and Machupo), hantavirus, smallpox (variola major), hemorrhagic fever virus, Nipah virus, and alphaviruses (e.g., Venezuelan equine encephalitis, eastern equine encephalitis, western equine encephalitis).

In another embodiment, the methods and compositions described herein may be used for promoting food freshness and/or combating or preventing food contamination. Examples of viral contaminants that may lead to foodborne illnesses include, for example, hepatitis A, norwalk-like viruses, rotavirus, astroviruses, calciviruses, adenoviruses, and parvoviruses.

In other embodiments, it may be desirable to administer or formulate the compositions of the invention in conjunction with other therapeutic agents. Exemplary therapeutic agents include, for example, anti-inflammatory agents, immunosuppressive agents, and/or anti-infective agents (such as for example, antibiotic, antiviral, and/or antifungal compounds, etc.). Exemplary anti-inflammatory drugs include, for example, steroidal (such as, for example, cortisol, aldosterone, prednisone, methylprednisone, triamcinolone, dexamethasone, deoxycorticosterone, and fluorocortisol) and non-steroidal anti-inflammatory drugs (such as, for example, ibuprofen, naproxen, and piroxicam). Exemplary immunosuppressive drugs include, for example, prednisone, azathioprine (Imuran), cyclosporine (Sandimmune, Neoral), rapamycin, antithymocyte globulin, daclizumab, OKT3 and ALG, mycophenolate mofetil (Cellcept) and tacrolimus (Prograf, FK506). Exemplary antibiotics include, for example, sulfa drugs (e.g., sulfanilamide), folic acid analogs (e.g., trimethoprim), beta-lactams (e.g., penacillin, cephalosporins), aminoglycosides (e.g., stretomycin, kanamycin, neomycin, gentamycin), tetracyclines (e.g., chlorotetracycline, oxytetracycline, and doxycycline), macrolides (e.g., erythromycin, azithromycin, and clarithromycin), lincosamides (e.g., clindamycin), streptogramins (e.g., quinupristin and dalfopristin), fluoroquinolones (e.g., ciprofloxacin, levofloxacin, and moxifloxacin), polypeptides (e.g., polymixins), rifampin, mupirocin, cycloserine, aminocyclitol (e.g., spectinomycin), glycopeptides (e.g., vancomycin), and oxazolidinones (e.g., linezolid). Exemplary antiviral agents include, for example, vidarabine, acyclovir, gancyclovir, valganciclovir, nucleoside-analog reverse transcriptase inhibitors (e.g., ZAT, ddI, ddC, D4T, 3TC), non-nucleoside reverse transcriptase inhibitors (e.g., nevirapine, delavirdine), protease inhibitors (e.g., saquinavir, ritonavir, indinavir, nelfinavir), ribavirin, amantadine, rimantadine, relenza, tamiflu, pleconaril, and interferons. Exemplary antifungal drugs include, for example, polyene antifungals (e.g., amphotericin and nystatin), imidazole antifungals (ketoconazole and miconazole), triazole antifungals (e.g., fluconazole and itraconazole), flucytosine, griseofulvin, and terbinafine.

In exemplary embodiments, the subject method is used to treat a subject who is infected with a human papillomavirus (HPV), particularly a high risk HPV such as HPV-16, HPV-18, HPV-31 and HPV-33. In other preferred embodiments, treatment of low risk HPV conditions, e.g., particular topical treatment of cutaneous or mucosal low risk HPV lesions, is also contemplated.

The subject method can be used to inhibit pathological progression of papillomavirus infection, such as preventing or reversing the formation of warts, e.g. Plantar warts (verruca plantaris), common warts (verruca plana), Butcher's common warts, flat warts, genital warts (condyloma acuminatum), or epidermodysplasia verruciformis; as well as treating papillomavirus-infected cells which have become, or are at risk of becoming, transformed and/or immortalized, e.g. cancerous, e.g. a laryngeal papilloma, a focal epithelial, a cervical carcinoma, or as an adjunct to chemotherapy, radiation, surgical or other therapies for eliminating residual infected or pre-cancerous cells.

In vitro and ex vivo uses are also contemplated herein. For example, an inhibitor of Brd4 complex formation, such as a portion of a Brd4 protein or an E2 protein or functional equivalent thereof, may be added to ex vivo or in vitro cells and tissues to, e.g., protect the cells from viral contamination or from spreading of a viral contamination. Cells and tissues treated in this manner may be used, e.g., for administering to a subject, such as in a graft transplant, or for analysis, such as forensic analysis. For example, a biopsy obtained from a subject may be treated as described to prevent contamination or spreading of a viral infection. Inhibitors of Brd4 complexes may also be added to blood in blood banks or to other cells.

Pharmaceutical compositions of this invention include any modulator identified according to the present invention, or a pharmaceutically acceptable salt thereof, and a pharmaceutically acceptable carrier, adjuvant, or vehicle. In an exemplary embodiment, pharmaceutical compositions of the invention will include a peptide or peptidomimetic of Brd4 that is capable of disrupting an interaction between Brd4 and an E2 protein or a functional equivalent of an E2 protein. In another embodiment, pharmaceutical compositions of the invention will include an anti-Brd4 and/or anti-E2 antibody that is capable of disrupting an interaction between a Brd4 protein and an E2 protein or a functional equivalent thereof. In yet another embodiment, the pharmaceutical compositions of the invention will include a nucleic acid encoding a Brd4 polypeptide wherein the polypeptide is capable of disrupting an interaction between a Brd4 protein and an E2 protein or a functional equivalent thereof. The term “pharmaceutically acceptable carrier” refers to a carrier(s) that is “acceptable” in the sense of being compatible with the other ingredients of a composition and not deleterious to the recipient thereof.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. The term parenteral as used herein includes subcutaneous, intracutaneous, intravenous, intramuscular, intra articular, intrasynovial, intrasternal, intrathecal, intralesional, and intracranial injection or infusion techniques.

Exemplification

The invention now being generally described, it will be more readily understood by reference to the following examples which are included merely for purposes of illustration of certain aspects and embodiments of the present invention, and are not intended to limit the invention in any way.

Some DNA viruses, like the papillomavirus and the lymphotropic herpesviruses, which establish persistent or latent infections, must maintain their genomes as stable episomes in dividing cells. Although elaborate mechanisms have been demonstrated for the effective segregation of low-copy-number plasmids in prokaryotes, the mechanisms by which eukaryotic episomal viruses ensure genome maintenance have not been yet fully elaborated. A major obstacle for maintaining plasmids in eukaryotes is presented by the breakdown and reassembly of the nuclear membrane during cell division. Noncovalent association with cellular chromosomes appears to be the principle strategy employed by episomal DNA viruses to ensure that their genomes are enclosed within the new nuclear envelopes and thus maintained in progeny cells.

The papillomavirus E2 is a multifunctional viral gene product that has been implicated in viral DNA replication, viral transcription, and regulation of cellular transformation. In addition, E2 protein has been shown to play a critical role in plasmid maintenance by linking the viral genomes to the cellular mitotic chromosomes to ensure their accurate segregation into daughter cells. However, the cellular factors that mediate E2 and host cell interactions remained largely unknown. To address this question, we employed a proteomic tandem affinity purification (TAP) approach to systematically analyze cellular proteins that associate with E2 in vivo. Mass spec analysis of the proteins co-purified with E2 has identified a cellular factor named Brd4. We cloned the full-length cDNA of the human form Brd4 and studied its role in the E2 functions.

Using co-immunoprecipitation, we showed that endogenous Brd4 interacts with both human and bovine papillomavirus E2 protein, suggesting a conserved role involving Brd4 in papillomavirus E2 function. Brd4 interacts specifically with the N-terminal transactivation domain of E2, and the E2 binding region on Brd4 has been mapped to its C-terminal region. Immunofluorescent analysis revealed the co-localization of E2 and Brd4 on mitotic chromosomes in human cells, suggesting that the Brd4 may represent the previously unidentified cellular factor that serves as the receptor of E2 on mitotic chromosomes. Expression of a truncated C-terminal domain of Brd4 inhibits the interaction of endogenous Brd4 with E2 and also prevents the co-localization of the viral protein and its cellular partner on mitotic chromosomes. Co-transfection of this dominant-negative truncation mutant of Brd4 with BPV-1 genome into C127 cells significantly inhibited the transformation efficiency of BPV-1.

We have further demonstrated that the colocalization of E2 with endogenous Brd4 on host mitotic chromosomes involves the N-terminal transactivation domain of E2. In addition, BPV-1 Fluorescence in Situ Hybridization (FISH) analysis showed that, using the E2 binding domain of Brd4, the C-terminal domain (CTD), as a dominant negative inhibitor, we could abolish the tethering of BPV1 DNA to host mitotic chromosomes in BPV1 transformed cells. Furthermore, quantitative analysis of viral episome levels in CTD-expressing cells that carry BPV-1 exclusively as episomes revealed a progressive loss of BPV-1 DNA with cell passage. Subcloning and morphology analysis of the H2 cells showed that 66% of the CTD-expressing cells reverted to the flat morphology typical of uninfected cells while a 13% rate of revertance was observed for vector control cells.

Taken together, our studies demonstrated that the cellular protein Brd4 plays an important role in tethering the papillomavirus genome to host mitotic chromosome through its interaction with E2 protein and may represent an important therapeutic target for papillomavirus infections.

The results and figures of Examples 1-12 are set forth in You et al. (2004) Cell 117:349, which is specifically incorporated by reference herein.

### EXAMPLE 1

### Tandem Affinity Purification with E2 Protein

To identify cellular factors that may play important roles in viral E2-host cell interactions, a proteomic tandem affinity purification (TAP) approach was employed to systematically analyze cellular proteins that associate with E2 in vivo. In this study, BPV E2TA protein (full length E2 from BPV1) was tagged with both FLAG and HA epitopes and stably expressed in the human cells. Since most of E2TA's biological functions have been assigned to its N-terminal transactivation domain, a truncation mutant of E2 missing the transactivation domain, E2TR (consisting of aas 162-410), was similarly tagged and expressed in cells as a negative control. SDS-PAGE electrophoresis of proteins co-purified with E2-TA or E2-TR identified a major protein that is uniquely present in the E2-TA pull down. Mass spec analysis of this E2-TA specific band has identified a cellular factor called Brd4 (bromodomain-containing protein 4).

### EXAMPLE 2

### Mouse Brd4 Western Blot of E2 Co-Immunoprecipitation

Brd4 contains two bromodomains (named after Drosophila protein brahma), a conserved sequence motif which may be involved in chromatin targeting. Brd4 was identified as the chromosome 19 target of translocation t(15;19)(q13;p13.1), which defines a lethal upper respiratory tract carcinoma in young people (CA French et al., Am. J. Pathology 159(6): 1987-1992 (2001)). The mouse homologue of Brd4, also called MCAP, has been shown to associate with chromosomes during mitosis and affect G(2)-to-M transition (A Dey et al., Mol. Cell. Biology 20(17): 6537-6549 (2000)). Ectopic expression of mouse Brd4 in NIH 3T3 and HeLa cells inhibits cell cycle progression from G(1) to S (T Maruyama et al., Mol. Cell. Biology 22(18): 6509-6520 (2002)). It has also been shown that, while Brd4 heterozygotes mice display pre- and postnatal growth defects associated with a reduced proliferation rate, mouse embryos nullizygous for Brd4 die shortly after implantation. In primary cell cultures, heterozygous cells also display reduced proliferation rates (D Houzelstein et al., Mol. Cell. Biology 22(11): 3794-3802 (2002)). These studies have suggested a fundamental role of Brd4 in cellular growth control and cell cycle progression.

To confirm the mass spec identification result, we used a rabbit Brd4 antibody (recognizes both human and mouse Brd4) to blot the protein samples co-immunoprecipitated with E2TA or E2TR after tandem FLAG and HA affinity purification. The mouse Brd4 western blot detected a major band of ˜200 Kd and two minor short fragments present in the E2TA pull down sample but not in the E2TR sample. This 200 Kd band is the expected full-length product of Brd4 and we believed that the two shorter fragments represent proteolytic-cleavage product of Brd4 since they were also detected in the Coomassie Blue stained gel for the mass spec analysis. The experiment confirmed that endogenous Brd4 specifically interacts with E2TA, but not E2TR, and suggested that the transactivation domain of E2 is critical for this interaction.

### EXAMPLE 3

### E2TA Pull Down of FLAG-MBrd4

Human C33A cells stably expressing either FLAG-HA-E2TA or FLAG-HA-E2TR were transiently transfected with FLAG tagged mouse Brd4 gene or an empty vector. Both cytoplasmic extract (CE) and nuclear extract (NE) were prepared from the transfected cells and immunoprecipitated (IP) with HA antibody to pull down E2 and associated proteins. The IP samples were analyzed by western blot using a Brd4 antibody. Among all the samples analyzed, only NE from cells expressing FLAG-HA-E2TA showed co-immunoprecipitation of Brd4 proteins. Three bands detected in one lane where cells were transfected with only an empty vector corresponded to endogenous Brd4 protein and its cleavage products that co-IP with E2TA. In cells transfected with FLAG tagged mouse Brd4 (FLAG-MBrd4) (lane 10), an additional set of bands migrating at slightly higher position were also detected, suggesting the co-IP of the FLAG tagged MBrd4 with E2TA. These experiments demonstrated that, in addition to the endogenous protein, transfected Brd4 protein also specifically interacts with E2TA through the transactivation domain.

### EXAMPLE 4

### HPV16E2 Interacts with HBrd4

After confirming the interaction of Brd4 protein with E2TA from BPV genome, we further addressed if Brd4 interacts similarly with human papillomavirus E2 such as HPV16 E2. C33A cells were transfected with FLAG-16E2 or an empty vector. CE and NE prepared from these cells were subjected to FLAG IP to pull down 16E2 and associated proteins. Brd4 antibody specifically detected a set of bands corresponding to the Brd4 and its proteolytic-cleavage products in the NE obtained from cells transfected with FLAG-16E2. No Brd4 bands were detected in the CE IP, nor was Brd4 immunoprecipitated in the NE of the cells transfected with only an empty vector. FLAG and 16E2 antibodies were used in western blot to show the IP of 16E2 protein in cells transfected with FLAG-16E2.

### EXAMPLE 5

### Cloning of Human Brd4

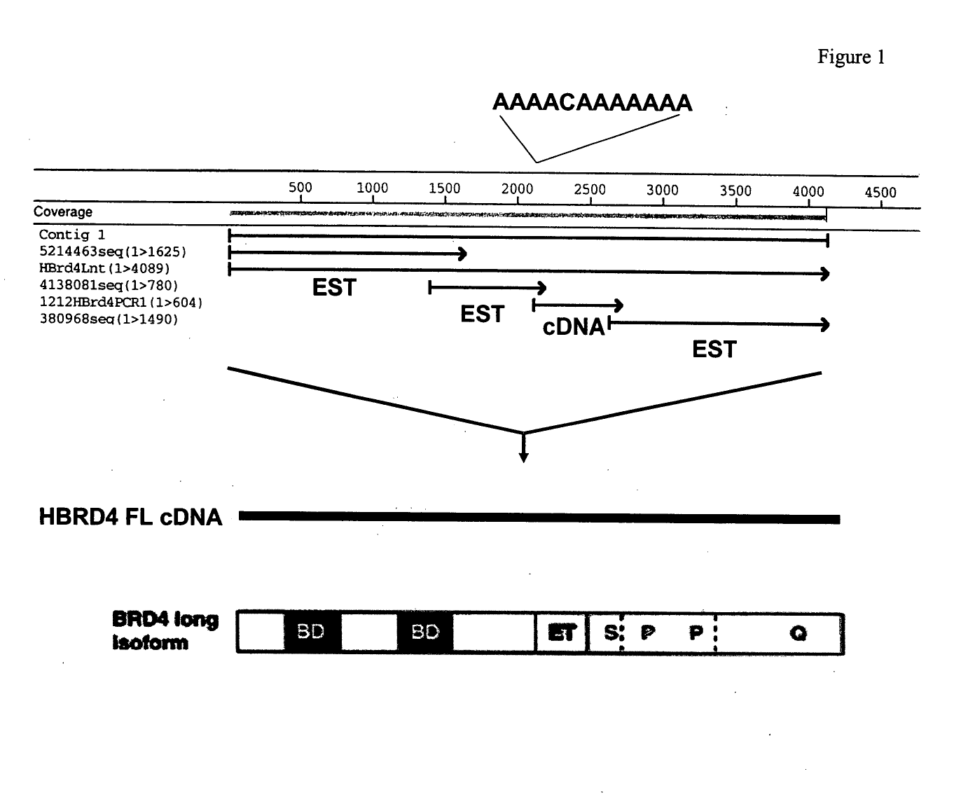

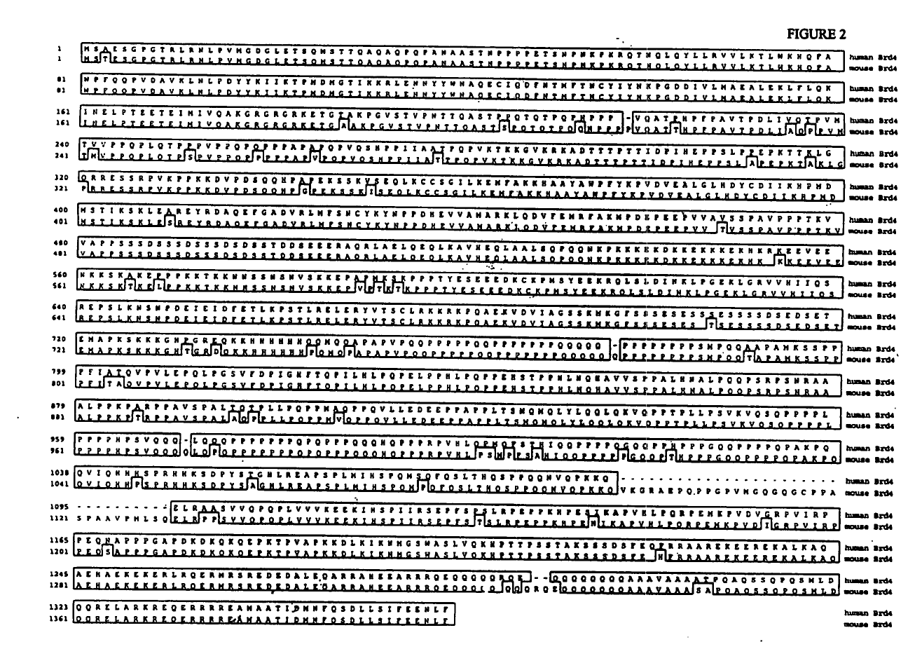

Human Brd4 cDNA was previously not available. The three EST clones we obtained from ATCC IMAGE bank only cover part of the predicted hBrd4 cDNA. The region of nt2000-2500 contains long repeats of polyA sequence and is therefore missing in all of the cDNA clones. This fragment was obtained from screening a human cDNA library. The full-length human Brd4 cDNA was subsequently constructed by ligating cDNA fragments together. A schematic of the cloning of human Brd4 is shown in FIG. 1. The nucleotide sequence for human Brd4 is set forth as SEQ ID NO: 1 and the nucleotide sequence for mouse Brd4 is set forth in SEQ ID NO: 3 (GenBank Accession number NM_{—}020508). An alignment between the amino acid sequences of human Brd4 (SEQ ID NO: 2) and mouse Brd4 (SEQ ID NO: 4) is shown in FIG. 2. The alignment was carried out using ClustalV and indicated a percent identity between the human and mouse Brd4 amino acid sequences of 94.6%.

### EXAMPLE 6

### Mapping of the E2 Binding Domain on Human Brd4

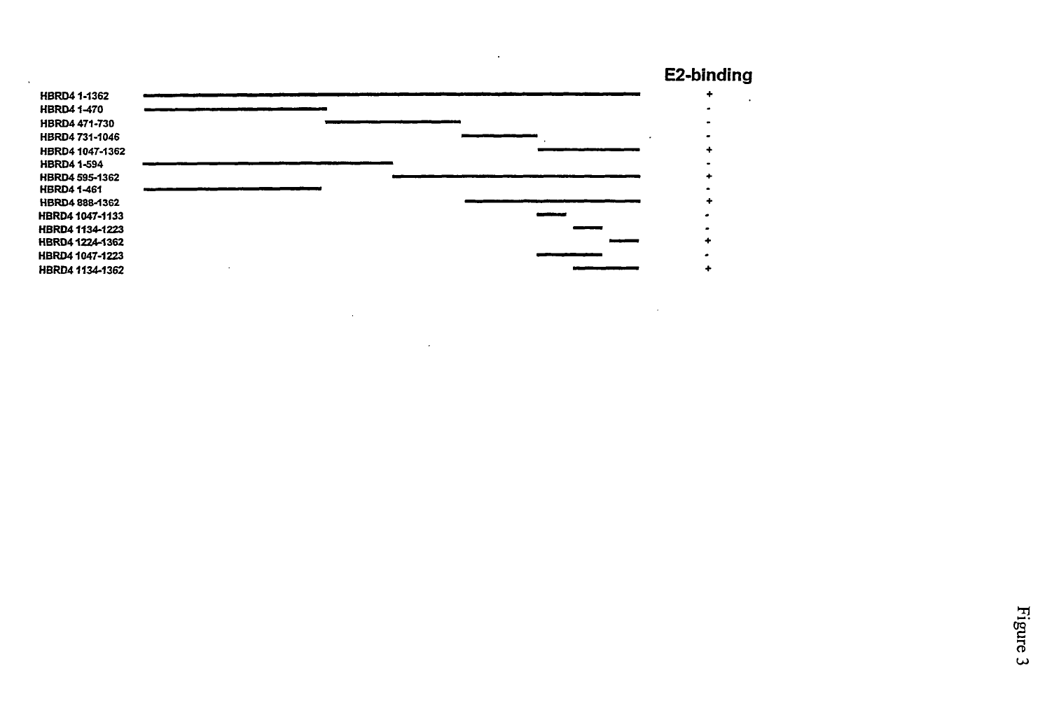

To map the E2 binding domain on human Brd4 protein, the hBrd4 cDNA fragments covering different functional domains of the protein were subcloned into an expression vector driven by T7 promoter. Each fragment was then translated and labeled by S35 using an in vitro transcription and translation (TNT) kit from Promega. Equal amount of each HBrd4 TNT product was then incubated separately with either GST-E2TA or GST-E2TR that has been immobilized on glutathione resin at 4° C. for 4 hours. After wash 4 times with binding buffer, the glutathione beads were eluted with SDS sample buffer. The results of this experiment are shown in FIG. 3. Equal amount of eluate from GST-E2TA and GST-E2TR were resolved on SDS-PAGE gel together with 30% of the input sample. The radioactive bands of the TNT products were detected by autoradiography. Since full-length Brd4 only binds to E2TA but not E2TR, the later GST fusion protein served as a negative control for this binding experiment. The fragments that showed significantly increased signal and had no higher than background signal were identified as E2-binding fragments. As shown in FIG. 3, all protein fragments containing the C-terminal residues 1047-1362 were able to specifically bind to E2TA protein, indicating that the E2 binding domain of hBrd4 resides in the 1047-1362 region.

We further subcloned the region encoding the C-terminal 300 amino acids of hBrd4 and used the TNT method to express them as approximately 100 a a fragments. By repeating the binding of GST-E2TA or E2-TR as described above, the E2 binding region was mapped to the last 138 amino acids of the hBrd4 protein.

### EXAMPLE 7

### Disruption of the hBrd4/E2 Interaction

We tested if expressing the C-terminal 1047-1362 region of hBrd4 in cells would disrupt the binding of Brd4 and E2. C33A cells stably expressing the FLAG-HA-E2TA protein were transiently transfected with either a pcDNA4c plasmid expressing His-Xpress-SV40NLS-HBrd41047-1362 product or an empty vector. 48 hours after transfection, cytoplasmic extract (CE) and nuclear extract (NE) were prepared from these cells and immunoprecipitated with anti-FLAG antibody. Brd4 antibody can detect the co-IP of the hBrd4 protein with E2 only in the NE of the cells transfected with an empty vector. Where the cells were transfected with the His-Xpress-SV40NLS-HBrd41047-1362 plasmid, the Brd4 antibody could no longer detect the bands corresponding to the full-length endogenous Brd4 protein and its proteolytic-cleavage products. Since the Brd4 antibody was raised against the last 14 aa of the Brd4 protein, it recognized the over-expressed His-Xpress-SV40NLS-HBrd41047-1362 product instead. This result demonstrated that the C-terminal 1047-1362 product of hBrd4, when expressed in human cells, could indeed disrupt the binding between E2 and Brd4 through competition effect, thus proving that this fragment can be used efficiently as an inhibitor for the E2-Brd4 binding in vivo. (The same bands of His-Xpress-SV40NLS-HBrd41047-1362 product were also detected by western blot using an anti-Xpress antibody in both the NE and CE sample, suggesting that the His-Xpress-SV40NLS-HBrd41047-1362 product may leak into CE during cell lysate fractionation).

### EXAMPLE 8

### Co-Localization of Brd4 and E2 on Mitotic Chromosomes

C33A-E2TA stable cells were double-stained with Brd4 antibody and E2TA antibody. The cells were also counter stained with DAPI to label nucleus and mitotic chromosome. In addition to the overall nucleus staining, the Brd4 antibody detected Brd4 protein present in highly condensed dots in the nucleus. E2 antibody also revealed both the nuclear staining of E2 and the high-density E2 staining dots, which have similar pattern as the Brd4 dots. Strikingly, both Brd4 and E2 staining dots were most distinctively observed on all the mitotic chromosomes. This result provided the first indication that E2TA and Brd4 protein co-localize in the dots on mitotic chromosomes.

### EXAMPLE 9

### HBrd4 C-terminal Fragment Blocks GST-E2TA Binding to Endogenous hBrd4

To test if hBrd4 c-terminus could prevent the recombinant GST-E2TA protein from binding to endogenous hBrd4 protein, C33A cells were treated with Streptolysin-O to allow entering of large molecules into the cells. The E. coli expressed GST-E2TA was pre-incubated for 15 min at room temperature with or without the recombinant His-HBrd41134-1362 before applying to the cells. After fixation and extraction, the cells were double stained for Brd4 and E2. The data showed that, in the absence of His-HBrd41134-1362, nucleus was stained with both E2 (green) and Brd4 (red) antibody. Pre-incubation of GST-E2TA with the inhibitor completely eliminated the E2 nuclear staining without affecting Brd4 staining. The result demonstrated that His-HBrd41134-1362 can bind to E2 and prevent the nuclear localization of E2TA.

### EXAMPLE 10

### HBrd4 C-Terminal Fragment Blocks E2TA Binding to Mitotic Chromosomes

C33A-E2TA stable cells were infected with retrovirus to generate cell line stably expressing His-Xpress-SV40NLS-HBrd41047-1362 or carrying an empty vector as a control. These cells were separately double-stained with Brd4 antibody (red) and E2TA antibody (green). Cells were also counter-stained with DAPI to label the nucleus and mitotic chromosome. In the E2 cells carrying an empty vector, the double-staining showed co-localization of E2 and Brd4 as high-density dots on mitotic chromosomes. Remarkably, in cells expressing His-Xpress-SV40NLS-HBrd41047-1362, the E2 staining was completely excluded from mitotic chromosomes, while the Brd4 staining on mitotic chromosomes was not affected. This result demonstrated that, by inhibiting the E2 and Brd4 interaction, HBrd41047-1362 prevented the tethering of E2 to host mitotic chromosomes.

### EXAMPLE 11

### Chromatin Immunoprecipitation (ChIP) Analysis of the Interaction Between hBrd4 and the BPV-1 Genome

C127 cells or C127 cells carrying BPV-1 extrachromosomal genomes (also called H2 cells) were used in this experiment. In addition, to block the Brd4 and E2 interaction, H2 cells were infected with retrovirus to generate cell line stably expressing His-Xpress-SV40NLS-HBrd41047-1362 (H2 I cells) or carrying an empty vector as a control (H2V cells). Cells were crosslinked in 1% para-formaldehyde for 10 min at room temperature. The fixed cells were washed in PBS, and chromatin was sonicated to an average DNA length of 600 bp. Chromatin DNA from 2e7 cells was incubated at 4° C. for 4 hr with 5 μg of Brd4 antibody or a control nonimmune normal rabbit IgG or without antibody. For “mock” chromatin IP, IP buffer were used instead of chromatin. Antibody complexes were recovered on Staphylococcus aureus protein A-positive cells, extensively washed with buffers and then eluted from the beads. Chromatin was de-crosslinked, and DNA was extracted with phenol/chloroform and ethanol precipitated. PCR using a pair of primers specifically amplifying a region of BPV-1 genome was performed to detect the BPV-1 DNA in the CHIP sample. The PCR products were separated by electrophoresis in 1.2% agarose gels, detected by ethidium bromide staining, and digitally photographed.

In contrast to the background signal shown in “no Ab” or “normal rabbit IgG” IP, the Brd4 antibody was able to specifically pull down BPV-1 genome in H2 cells as well as H2V cells (not in C127 cells because these cells don't have BPV-1 episomes). In cells stably expressing the inhibitor, HBrd41047-1362 (see H21 cells), the amount of IPed BPV-1 as detected by the PCR reduced to background level. This data shows that Brd4 can bind to BPV-1 genome through its interaction with E2. By blocking the E2-Brd4 interaction (with the expressed inhibitor), we can disrupt the tethering of BPV-1 genome by Brd4.

### EXAMPLE 12

### HBrd4 1047-1362 Inhibits the Transformation of C127 Cells by BPV-1

Mouse C127 cells were infected with retrovirus to generate cell line stably expressing His-Xpress-SV40NLS-HBrd41047-1362 (C127+I) or carrying an empty vector as a control (C127+V). C127 and the stable cells were transfected with BPV-1 genome. After 14 days, cell foci were fixed and stained with methylene blue. As shown in the figure, BPV-1 induced cellular transformation was demonstrated by the blue foci formed in the dish. In the presence of stable expression of HBrd41047-1362 (see C127+I cells), the cell foci number was dramatically decreased, suggesting that this molecule can inhibited the transformation potency of the BPV-1 virus genome. The results from three independent transfections into each cell line are summarized below in Table 1.

TABLE 1
Summary of Colony Formation Assay.

| Cell | Foci/1 μg BPV1 | Average | Standard Deviation |
|---|---|---|---|
| C127 | 150 | 150.3 | 4.5 |
| | 146 | | |
| | 155 | | |
| C127 + V | 141 | 136.0 | 7.8 |
| | 140 | | |
| | 127 | | |
| C127 + I | 8 | 9.3 | 1.2 |
| | 10 | | |
| | 10 | | |

### EXAMPLE 13

### Colocalization of E2 with Brd4 on Mitotic Chromosomes Involves the N-Terminal Transactivation Domain of E2

C33A, C33A/E2TA or C33A/E2TR stable cells were double-stained with an anti-Brd4 antibody and an anti-BPV-1 E2 antibody. The anti-Brd4 antibody is directed to the N-terminus of the protein. Cells were also counter-stained with DAPI to label the nucleus and mitotic chromosomes. In cells stably expressing E2TA, E2 and Brd4 colocalized in densely staining dots on mitotic chromosomes. Remarkably, E2TR (which lacks the N-terminal transactivation domain required for interaction with Brd4) was completely excluded from mitotic chromosomes in metaphase cells while Brd4 remained associated with the mitotic chromosomes. The Brd4 mitotic chromosome localization is similar in cells whether or not there is expression of E2 or E2TR. These results indicate that the colocalization of E2TA with Brd4 in punctate dots on mitotic chromosomes requires the E2 transactivation domain and confirms our biochemical findings that human Brd4 protein specifically interacts with E2TA and not with E2TR.

### EXAMPLE 14

### Stable Expression of the Brd4 C-terminal Domain Abrogates Association of the BPV-1 Genome with Host Mitotic Chromosomes

To address the affects of Brd4 C-terminal domain (CTD) on the association of BPV-1 genome with host mitotic chromosome, we used the C127C1H2 cells, which are BPV1 transformed mouse C127 cells that carry BPV1 DNA exclusively as episomes. These cells were transduced with retroviruses expressing the dominant negative inhibitor His-Xpress-SV40NLS-hBrd4-CTD or vector alone to generate stable cell lines H2-CTD or H2-V (FIG. 16). Stable expression of the Brd4-CTD was verified in the H2 cells by immunofluorescent staining using anti-Xpress antibody and we were able to demonstrate CTD expression in 90-95% of the H2-CTD cells.

H2 cells stably expressing Brd4-CTD (H2-CTD) or transduced with an empty retrovirus vector (H2-V) cultured in chamber slides were arrested at metaphase by a 2-hr incubation with Colcemid. Cells were lysed with hypotonic solution (0.56% KCl) and fixed to glass slide using Carnoy's fixative (75% methanol and 25% acetic acid) before hybridization with a BPV-1 probe in Fluorescence in Situ Hybridization (FISH) analysis. The BPV-1 probe was labeled in red. Cells were also counter-stained with D API to label the nucleus and mitotic chromosomes (in blue). The FISH result showed that the BPV-1 episomes that were readily detected associated with mitotic chromosomes in the H2-V cells, were undetectable in the H2-CTD cells. The data demonstrated that CTD expressed in the H2 cells completely abolished the association of BPV-1 episomes with mitotic chromosomes. For the H2-V cells, in 15 of 15 mitotic spreads, the metaphase chromosomes were positive for BPV-1 DNA by FISH. In contrast, for the H2-CTD cells, all of 15 sets of metaphase chromosomes analyzed were negative for BPV-1 DNA. This result demonstrated that, by blocking the E2/Brd4 interaction using CTD, we could specifically disrupt the association of BPV-1 genome to host mitotic chromosomes in cells stably maintaining viral episomes, confirming that the virus episome-chromosome interaction is mediated by E2 and Brd4.

### EXAMPLE 15

### Real-Time PCR Quantitative Analysis of BPV-1 Episomes in H2 Stable Cells

H2 cells were used to investigate whether stable expression of Brd4-CTD could lead to curing of infected cells, e.g., the elimination of viral episomes and morphologic reversion of transformed cells. H2 cells are mouse C127 cells transformed by BPV-1 that carry BPV-1 exclusively as episomes. Both vector control cell line H2-V and the Brd4-CTD expressing cell line H2-CTD were grown for the indicated number of passages (e.g., passage 1=P1) and split at a ratio of either 1:100 or 1:10 in different experiments. Total cellular DNA was extracted from the cultures at each passage and assayed for the quantity of BPV-1 DNA by real time PCR. We also analyzed the cells during the passage for their morphology and their ability to form colony.

To look at the dynamics of viral DNA loss in cell expressing the CTD, we carried out a time course experiment examining the levels of BPV-1 DNA by real-time PCR in CTD expressing H2 cells compared to vector control H2 cells. After retrovirus infection, H2 cells stably expressing Brd4-CTD (H2-CTD) or transduced with an empty retrovirus vector (H2-V) were cultured for the indicated number of passages and split at a ratio of 1:100. Total cellular DNA was extracted from the cultures at each passage and assayed for the quantity of BPV-1 DNA using a LightCycler (Roche). The concentration of viral DNA in each sample was calculated using the LightCycler Software version 3.5 based on a standard curve generated using known amounts of BPV-1 plasmid DNA. 250 pg of total cellular DNA from passage #1H2-V cells contains 0.21 pg of BPV-1 and the same amount of total cellular DNA from passage #1H2-CTD cells contains 0.24 pg of episome. The BPV-1 DNA content of each culture is presented as a percentage of the BPV-1 DNA from passage 1 of the same cell line. Our result indicate that during the first 2 passages, H2-CTD cells showed similar amounts of BPV-1 DNA as the vector control H2-V cells. Furthermore, by passage 3, the loss of viral DNA could be observed, and with continued passage, the H2-CTD cells, but not the H2-V cells, show progressive loss of BPV-1 DNA. By passage 5, there is a 78% loss of the BPV-1 DNA from the CTD expressing cells.

We looked at the morphology of H2-V cells and H2-CTD cells after 12 passages split at 1:10 dilution. The H2-V cells still maintained the transformed morphology (narrow and long shape cells with sharp edge). These transformed cells have lost the contact inhibition and, therefore, can grow to high saturation density. In contrast, the majority of the H2-CTD cells were reverted to the flat cellular morphology typical of uninfected C127 cells. These cells can only grow as monolayer. The high frequency of revertance in the H2-CTD cells after cell passage indicated a loss of resident viral genomes in the cells, confirming the real-time PCR result.

### EXAMPLE 16

### Morphology Analysis of CTD Expressing H2 Cells

To calculate the frequency of revertance in the stable cells, the H2-V and H2-CTD cells were cultured for 9 passages at 1:10 dilution and cloned into 96 well plates. Among the 30 single clones isolated for the H2-V cell line, 4 showed the revertant morphology. However, 12 out of the 18 single clones isolated from the H2-CTD culture showed the revertant morphology. The transforming clones have long and narrow shape cells that can grow to high cell density to form colonies (or foci). The revertants have round shape flat-looking cells that can only grow as monolayer due to the contact inhibition. Each type of clones was subcultured. The results demonstrate that the expression of Brd4 CTD leads to an increased frequency (from 13% for H2-V cell line to 66% for H2-CTD cell line) of revertance, suggesting a loss of BPV-1 viral genomes in the H2-CTD cells.

### EXAMPLE 17

### Inhibition of E2 Binding to Brd4 Enhances Viral Genome Loss and Phenotypic Reversion of Bovine Papillomavirus Transformed Cells

The bovine papillomavirus E2 protein tethers the viral genomes to mitotic chromosomes in dividing cells through binding to the C-terminal domain (CTD) of Brd4. Expression of the Brd4-CTD competes the binding of E2 to endogenous Brd4 in cells. Here we extend our previous study that identified Brd4 as the E2 mitotic chromosome receptor to show that Brd4-CTD expression released the viral DNA from mitotic chromosomes in BPV-1 transformed cells. Furthermore, stable expression of Brd4-CTD enhanced the frequency of morphological reversion of BPV-1 transformed C127 cells resulting in the complete elimination of the viral DNA in the resulting flat revertants. The text and figures of this Example are set forth n You et al. (2005) J. Virol. 79:14956, which is specifically incorporated by reference herein.

Introduction

The papillomaviruses are a group of small DNA viruses that cause benign lesions in higher vertebrates, including humans. The “high-risk” human papillomaviruses (HPVs) are associated with a number of human cancers including cervical cancer (21). The papillomaviruses have a specific tropism for squamous epithelial cells and infect cells within the basal epithelial layer to establish an infection. Late gene expression, lytic DNA amplification and virus production are restricted to the more terminally differentiated cells of the epithelium (9).

During the life cycle of the papillomaviruses, the viral DNA is maintained as an extrachromosomal plasmid at a low copy level in infected cells (9). Mouse cells transformed by BPV-1 maintain the viral DNA in a stable extrachromosomal plasmid state and have served as an excellent model for studying viral DNA replication and genome maintenance (8, 12, 14). The maintenance of the transformed phenotype requires the continued presence of viral genomes; Cells cured of the viral genomes revert to a flat, non-transformed phenotype (17).

To ensure that the viral genomes are not lost upon breakdown and reassembly of the nuclear membrane during cellular mitosis, papillomaviruses, like Epstein-Barr virus and Kaposi's sarcoma-associated herpesvirus, employ strategies to maintain their genomes in the nuclear space through the non-covalent association of their genomes to cellular mitotic chromosomes via a virally encoded DNA binding protein (2, 10, 11, 16).

Papillomaviruses genome maintenance has been best studied for BPV-1 (3, 11, 13, 15, 16). The persistence of the viral genomes is mediated through the multiple E2-binding sites of BPV-1 genome (15). E2 binds these specific sites through its DNA binding domain, and tethers BPV-1 DNA to mitotic chromatin in dividing cells through its transactivation domain (3, 13, 16). E2 mutations abrogating the mitotic chromosome attachment lead to the dramatic loss of viral genomes from BPV-1 transformed cells (13). Mutations in the transactivation domain have also been shown to disrupt the tethering of viral genomes to mitotic chromosomes (1, 4, 20).

Our previous work identified the bromodomain-containing protein 4 (Brd4) as the chromosome associated protein through which E2 and the viral DNA bind mitotic chromosomes (19). Brd4 is a member of the BET family proteins and associates with mitotic chromosomes during mitosis (6, 7). E2 binds Brd4 through the C-terminal domain (CTD) of Brd4. The Brd4-CTD can be stably expressed in cells where it inhibits the binding of E2 to endogenous Brd4 on mitotic chromosomes, prevents the tethering of BPV-1 DNA to Brd4 and blocks BPV-1 transformation of mouse C127 cells (19). Additional evidence has confirmed the role of Brd4 as the tether for E2 and viral genomes on mitotic chromosomes (4, 5).

In this study, we have further examined the functional significance of E2-Brd4 interaction in BPV-1 genome maintenance in BPV-1 transformed H2 cells and tested the potential of the Brd4-CTD to cure BPV-1 transformed cells of viral DNA.

E2 and Brd4 Bind Directly. We previously showed that Brd4 interacts with E2 in cells to form a molecular bridge linking the papillomavirus genomes to host mitotic chromosomes (19). However, we did not establish whether the E2 and Brd4 interaction was directly or mediated by an intermediate factor. We therefore tested whether their binding is direct.

GST fusion proteins were produced in E. coli. The fusion proteins, purified and immobilized on glutathione resin, were eluted by SDS sample buffer, resolved on SDS-PAGE and analyzed by commassie blue staining as input control. The Brd4-CTD fragment was produced in E. coli from the pET32a plasmid encoding the His tagged Brd4-CTD. After purification over a Ni-NTA column, the tag was cleaved with Enterokinase. 10 μg of Brd4-CTD was mixed with 5 μl of immobilized GST-E2 and the binding was performed as described previously (19). Eluates from GST-E2 beads were resolved by SDS-PAGE along with 60% of the Brd4-CTD input, and detected by Western blot using a Brd4 antibody C-MCAP (7).

Purified recombinant Brd4-CTD was incubated with purified GST fusion proteins containing HPV-16E2, BPV-1 E2TA (full length E2) or BPV-1 E2TR (truncated E2 lacking the transactivation domain) immobilized on glutathione beads. E2TR that does not bind endogenous Brd4 served as a negative control (19). The smaller fragments observed represent the proteolytic cleavage products of Brd4-CTD. They are still recognized by the antibody against the C-terminal 14 aa of Brd4, suggesting that the cleavages occurred near the N-terminus of the Brd4-CTD. In consistent with our previous result showing that the E2-binding domain can be further mapped to the last 138 amino acids (aa 1224-1362) of Brd4, these C-terminal fragments of Brd4-CTD all bound to E2. Brd4-CTD bound efficiently to both GST-16E2 and GST-E2TA, but not to GST-E2TR, demonstrating that the binding between E2 and Brd4 is direct.

Brd4-CTD Dissociates BPV-1 Viral Genomes from the Host Mitotic Chromosomes. Brd4-CTD can abolish the Brd4/mitotic chromosome association of E2 as well as the tethering of BPV-1 DNA to Brd4 (19). We therefore tested whether Brd4-CTD could dissociate the viral DNA from host mitotic chromosomes in H2 cells, a clonal line of C127 cells harboring exclusively extrachromosomal BPV-1 DNA. H2-CTD and H2-V cell lines were established previously by transduction of retroviruses expressing either the Xpress-tagged Brd4-CTD or empty vector (19).

Passage 3H2-CTD and H2-V cells were cultured in chamber slides and arrested at metaphase by a 2-hr incubation with 1 μg/mL Colcemid. Cells were lysed with hypotonic solution (0.56% KCl) and fixed to glass slides using Carnoy's fixative (75% methanol and 25% acetic acid) before hybridization with a BPV-1 probe labeled in red using Fluorescence labeling reagents from Vysis. Cells were also counter-stained with Vysis DAPI II anti-fade and examined using an Olympus AX70 microscope with red-green-DAPI filters and Genus software from Applied Imaging.

Immunofluorescent staining using anti-Xpress antibody verified that Brd4-CTD was expressed in ˜90% of H2-CTD cells. As expected, BPV-1 DNA was detected as punctuate dots associated with host mitotic chromosomes in H2-V cells (13, 16). However, the FISH signal was not detected in H2-CTD cells. In each of the 15H2-V mitotic spreads examined, metaphase chromosomes were positive for BPV-1 DNA. In contrast, for H2-CTD cells, all 15 metaphase chromosome spreads analyzed were negative for BPV-1 DNA. This result demonstrated that, in blocking the E2-Brd4 interaction, the Brd4-CTD efficiently disrupts the association of BPV-1 DNA with mitotic chromosomes, further confirming that the viral genome-host chromosome interaction is mediated by E2/Brd4 binding. The cells analyzed were at passage 3 after retrovirus transduction. As described below, the H2-CTD cells at passage 3 and at later passages still contain BPV-1 DNA. Therefore, the lack of any detectable DNA associated with the mitotic chromosomes in these cells reflects the fact that the viral genomes, while still present in the cell, are no longer tightly associated with mitotic chromosomes. The dissociated genomes are presumably washed away by the hypotonic washes and the Carnoy's fixative used in the FISH procedure.

Brd4-CTD Induces Morphologic Reversion in H2 Cells. The FISH data predicted that Brd4-CTD expression might lead to the curing of the extrachromosomal DNA from H2 cells since they were no longer tightly associated with host mitotic chromosomes. To address this question, both H2-CTD and H2-V cells were continuously split at 1:10 ratio. In early passage cells, there were no obvious morphologic differences between the control and CTD-treated cells. At passage 4, however, we observed some flat cells resembling non-transformed parental C127 cells in the H2-CTD culture but not in H2-V cells. This flat phenotype became more evident with continued passage. By passage 12, the majority of the Brd4-CTD-expressing cells showed a non-transformed morphology with only occasional transformed cells intermingled among the flat cells, whereas the H2-V cells retained the transformed morphology throughout the analysis. Therefore Brd4-CTD expression led to a progressive reversion from the transformed phenotype to a flat cell morphology resembling the parental C127 cells. Furthermore stable expression of the Brd4-CTD had no effect on the morphology or growth characteristics of non-transformed C127 cells, HeLa cells or C33A cells.

CTD Lowers the BPV-1 Plasmid Number in H₂Cells. To test whether the phenotypic reversion observed in H2-CTD cells after multiple cell passages results from the loss of viral DNA in these cells, we compared the levels of BPV-1 DNA in H2-CTD and H2-V cells at each passage. Real-time PCR analysis for BPV-1 genome demonstrated that the BPV-1 DNA copy numbers remained nearly unchanged for the first three passages in both cell lines. At passages 3-6, the H2-V cells maintained approximately 35±14 copies of viral DNA/cell, compared to the 40 copies/cell for H2 cells. Despite some experimental variability, there was no significant trend of DNA reduction detected in H2-V cells. In contrast, analysis of the same passages from H2-CTD cells showed a dramatic decrease of BPV-1 DNA level at passages 4 and higher. By passage 6, the relative abundance of viral DNA in H2-CTD cells was reduced to approximately 25% of the level in H2-V cells (or approximately 8 copies/cell). This decrease in BPV-1 DNA in H2-CTD cells after passage 3 was confirmed by the Southern blot analysis (data not shown). The timing of the significant genome loss in H2-CTD cells at passage 4 coincided well with the initial appearance of flat revertants in cell culture, suggesting that the flat H2-CTD cells were due to the loss of viral DNA in these cells.

This influence of Brd4-CTD on the transformed cell morphology and viral genome level was observed several passages (≧4) after the CTD was tranduced into the cells. This result is consistent with our previous ChIP result showing that Brd4-CTD expression in H2 cells did not cause a significant dilution/loss of the viral DNA in a single passage, but did dissociate the viral DNA from Brd4 (19). With continued passage, some of the Brd4-CTD-expressing cells may eventually lose the viral genomes and revert to a non-transformed phenotype. We have recently found that the E2 transcriptional activation function is also dependent upon Brd4 and that the Brd4-CTD inhibits this function (M.-R.S., J.Y. and P.M.H., manuscript submitted). Whereas it is possible that the inhibition of E2 transcriptional activation by Brd4-CTD could contribute to the morphologic reversion of H2 cells, previous studies have shown that BPV-1 genomes mutated for the E2 transactivation function are still transformation competent (18). Thus, the nearly complete dissociation of viral DNA from host mitotic chromosomes at the early stage of Brd4-CTD expression as shown by FISH analysis argues strongly that disrupted tethering is responsible for the loss of viral genomes and the resulting morphologic reversion in the cells. The ability of Brd4-CTD to completely dissociate viral plasmid from mitotic chromosomes makes it likely that Brd4 might be the sole receptor for BPV-1 E2 and viral DNA during mitosis.

Colony Morphology Analysis of Brd4-CTD induced Reversion. Although we detected a significant decrease of BPV-1 DNA levels in passage 4-6 of H2-CTD cells, the DNA content leveled off after passage 6. We speculated that the continued culture of H2-CTD cells would provide a select advantage for transformed cells harboring BPV-1 DNA. To establish a direct link between Brd4-CTD-mediated viral genome loss and morphologic reversion, we examined H2-CTD cells at a single-cell level by analyzing colony morphology. H2-CTD and H2-V cells from each passage were plated as single cells at 20-30 cells per plate and cultured for 20 days to evaluate the morphology of colonies derived. After staining with methylene blue as described (19), transformed colonies that were not contact inhibited and grew to high cell density stained dark blue. In contrast, flat revertants, which form a contact inhibited monolayer, stained light blue. Interestingly, a third population of colonies containing both light and dark blue staining, hence termed “mix clones”, was also observed. In contrast to the homogenous populations of cells in the transformed and flat colonies, the mixed colonies had both cell types. We examined ˜100 colonies for each cell line at each passage for 10 passages and quantitated each colony type. For H2-CTD cells, the majority of the colonies were either flat (32%) or mixed (62%) and only 6% of the colonies were fully transformed, whereas for H2-V cells, 50% of the colonies retained a fully transformed morphology.

Notably, a large percentage of the colonies derived from the H2-CTD cell line showed mixed colony morphology. In some cases, only the cells in the middle of the colony retained the transformed morphology, whereas in others transformed cells form a pie-shape patch emanating from the center of the colonies. The sectoring was not a function of cell plating density because H2-V cells plated at the same density gave rise to a much lower number of such colonies. Rather, these sectoring patterns were reminiscent of the plasmid sectoring phenotype of a BPV-1 mutant in which the plasmid segregation is compromised by an E2 mutation (13). This sectoring pattern suggested a plasmid maintenance defect in H2-CTD cells where the BPV-1 plasmids are no longer tightly associated with host chromosomes. We reasoned that the sectored colonies arose by an asymmetrical distribution of BPV-1 molecules to daughter cells.

At passage 11, single-cell-clones were also isolated by cloning into 96 well plates. Among 30 single clones isolated from H2-V cell line, 4 showed a completely flat morphology and the others were either mixed or fully transformed. In contrast, 12 out of 18 single clones isolated from H2-CTD culture showed the revertant flat morphology. Immunofluorescence staining of Brd4-CTD in H2-CTD cells showed that, while 80% of the cells still expressed the Brd4-CTD at passage 4, only 5% of the cells at passage 6 and less than 1% of the cells at passage 10 were positive for Brd4-CTD expression. Therefore, these data suggested that the frequency of revertants continued to increase even after the loss of Brd4-CTD expression, perhaps reflecting inefficient partitioning of the viral genomes once the copy number was reduced. The reduction of Brd4-CTD expression might be a factor that limited the capacity of Brd4-CTD to “cure” viral genomes in all of the cells treated. As the Brd4-CTD expression is lost with passage, it is expected that E2 would regain its ability to bind Brd4 and mitotic chromosomes. Nonetheless, this morphologic analysis of the individual colonies and clones demonstrated that the Brd4-CTD expression significantly enhanced the reversion of transformed H2 cells to a flat non-transformed phenotype.

Phenotypic Reversion Resulted From the Loss of Viral Genomes. We next tested whether the flat cell reversion seen in H2-CTD cells was due to the loss of viral genomes. Independent colonies from passage 11 were expanded to determine whether the cells still harbored BPV-1 DNA. The clones derived from the H2 expressing the Brd4-CTD “inhibitor” were labeled either as “IT” for transformed or as “IF” for flat revertant. Similarly, the clones derived from H2-V cells carrying the retrovirus “vector” were labeled as “VT” and “VF” depending upon their morphology. Immunofluorescent staining of Brd4-CTD in the isolated “IF” or “IT” clones showed that none of these cells retained Brd4-CTD expression in agreement with our analysis that Brd4-CTD expression was lost during cell passaging. The fact that the reverted morphology persisted even after the Brd4-CTD expression was lost indicated that the reversion was due to the permanent loss of the viral genome rather than an effect of the Brd4-CTD on either cellular or viral gene expression.

We next analyzed the viral DNA levels in the isolated clones. Total cellular DNA was extracted and assayed for the presence of BPV-1 genomes by Southern hybridization as in (12). 20 μg of DNA was cleaved with SalI and the resulting fragments were separated on a 0.8% agarose gel, denatured and transferred to a Hybond N+membrane. Cloned BPV-1 DNA was labeled using Prime-It Random Primer Labeling Kit (Stratagene) and hybridized to DNA immobilized on the membrane. After washing, the filters were exposed to Kodak BMS film. FO I and FO II, supercoiled and nicked circular extrachromosomal DNA, respectively. FO III, linear viral DNA.

Total cellular DNA was digested with SalI (recognizes no sites in BPV-1 DNA) before Southern hybridization using a BPV-1 probe as described in (12). Noo BPV-1 DNA was detected in C127 cells or any of the flat revertant clones. In the transformed cell lines and in H2 cells, viral DNA was detected in its circular extrachromosomal forms (12). Some of the viral DNA was also converted to full-length linear DNA due to mild shearing of the DNA. This result was confirmed by Southern blot analysis with the single-cut enzyme BamHI. Both data suggested that, like H2 cells, the transformed cells harbored the viral DNA in an extrachromosomal state.

We also quantitated the viral genome level in H2-CTD clones by real-time PCR. Real-time PCR quantitation of the viral genomes in both transformed and reverted clones isolated from H2-CTD cell line was performed as follows. Cellular DNA was extracted from the cells using Hirt's lysis buffer (0.6% SDS and 10 mM EDTA, pH8.0) at 4° C. for 15 min followed by extraction in 1M NaCl at 4° C. overnight. After centrifugation at 14 k×g for 30 min at 4° C., the supernatant was analyzed using a LightCycler machine (Roche) according to the manufacturer's instructions. The PCR primers used in the analysis were designed to amplify a 432 bp region spanning nt. 2601-3032 of BPV-1 DNA.

Neither parental C127 cells nor flat revertants contained detectable BPV-1 DNA under conditions sensitive enough to detect 0.1 viral genome per cell. The “IT” clones showed some reduction (up to 50%) in the BPV-1 level compared to the parental H2 cells. This lower level of DNA suggested that the initial Brd4-CTD expression in H2-CTD cells may have contributed to a reduction of the viral DNA levels. This analysis thus established a direct correlation between viral DNA loss and phenotypic reversion of BPV-1 transformed cells.

Previously we showed that by blocking E2/Brd4 interaction, Brd4-CTD can inhibit BPV-1 transformation of C127 cells (19). In this study, we show that the Brd4-CTD reduces the BPV-1 genome levels in transformed cells, underscoring the role of E2/Brd4 association in the papillomavirus plasmid maintenance. The ability of the Brd4-CTD to cure infected cells of the PV genomes suggests that targeting E2/Brd4 binding might represent a new strategy for the development of papillomavirus antivirals. BPV-1-transformation provides an excellent model for analyzing plasmid maintenance and for investigating antiviral compounds.

### REFERENCES

1. Abroi, et al. (2004) J Virol 78:2100-2113. 2. Ballestas, et al. (1999) Science 284:641-644. 3. Bastien, et al. (2000) Virology 270:124-134. 4. Baxter, et al. (2005) J Virol 79:4806-4818. 5. Brannon, et al. (2005) Proc Natl Acad Sci USA 102:2998-3003. 6. Dey, et al. (2003) Proc Natl Acad Sci USA 100:8758-8763. 7. Dey, et al. (2000) Mol Cell Biol 20:6537-6549. 8. Dvoretzky et al. (1980) Virology 103:369-375. 9. Howley et al. (2001) Fields Virology, 4 ed, vol. 2. Lippincott Williams & Wilkins, Philadelphia. 10. Hung, et al. (2001) Proc Natl Acad Sci USA 98:1865-1870. 11. Ilves, et al. (1999) J Virol 73:4404-4412. 12. Law, et al. (1981) Proc Natl Acad Sci USA 78:2727-2731. 13. Lehman, et al. (1998) Proc Natl Acad Sci USA 95:4338-4343. 14. Lowy, et al. (1980) Nature 287:72-74. 15. Piirsoo, et al. (1996) EMBO 15:1-11. 16. Skiadopoulos, et al. (1998) J Virol 72:2079-2088. 17. Turek, et al. (1982) Proc Natl Acad Sci USA 79:7914-7918. 18. Vande Pol, et al. (1995) J Virol 69:395-402. 19. You, et al. (2004) Cell 117:349-360. 20. Zheng, et al. (2005) J Virol 79:1500-1509. 21. zur Hausen, et al. (2002) Nat Rev Cancer 2:342-350.

### EXAMPLE 18

### Bromodomain Protein 4 Mediates the Papillomavirus E2 Transcriptional Activation Function

The papillomavirus E2 regulatory protein has essential roles in viral transcription, the initiation of viral DNA replication as well as for viral genome maintenance. Brd4 has recently been identified as a major E2-interacting protein and, in the case of the bovine papillomavirus (BPV1) serves to tether E2 and the viral genomes to mitotic chromosomes in dividing cells, thus ensuring viral genome maintenance. We have explored the possibility that Brd4 is involved in other E2 functions. By analyzing the binding of Brd4 to a series of alanine scanning substitution mutants of the HPV16 E2 N-terminal transactivation domain, we found that amino acids required for Brd4 binding were also required for transcriptional activation but not for viral DNA replication. Functional studies in cells expressing either the C-terminal domain (CTD) of Brd4 that can bind E2 and compete its binding to Brd4 or siRNA to knockdown Brd4 protein levels, revealed a role for Brd4 in the transcriptional activation function of E2 but not for its viral DNA replication function. Therefore, these studies establish a broader role for Brd4 in the papillomavirus life cycle than as the chromosome tether for E2 during mitosis.

Introduction

The papillomaviruses (PV) are small DNA viruses that are etiologic agents for papillomas and warts in a variety of higher vertebrates, including humans. Specific human papillomaviruses (HPVs) have been associated with some human cancers, most notably cervical cancer (52). The papillomaviruses establish long term, persistent infections of squamous epithelial cells and the viral life cycle is tightly linked with the differentiation program of the host cell (20). In the infected dividing basal cells of the epithelium, the viral DNA is maintained as a stable plasmid. Vegetative viral DNA replication occurs only in the more differentiated squamous epithelial cells. The bovine papillomavirus (BPV) DNA remains extrachromosomal in transformed rodent cells, a system that has served as a useful model for studying viral genome maintenance (27).

The papillomavirus E2 protein has important roles in regulating viral transcription, in enhancing E1 dependent viral DNA replication and in genome maintenance (20). E2 is a DNA binding protein that was first identified as a transcriptional activator (43). Subsequent studies established that E2 can also repress some genes, depending upon the location of its cognate binding sites within the promoter region (44). Indeed, E2 functions to repress the promoter directing the E6 and E7 viral oncogenes in the cancer associated HPV16 and HPV18 genome (37). For viral genome replication, E2 binds the viral helicase E1 and guides it to the origin of replication in the process of initiating origin dependent viral DNA replication (6, 33). For genome maintenance, E2 has been shown to associate with mitotic chromosomes and in doing so to anchor the viral genomes to the host chromosomes during mitosis (4, 21, 30, 34, 42). The structure of E2 resembles that of a prototypic transcription factor, with an amino terminal transcriptional activation (TA) domain and a carboxy terminal DNA binding and dimerization domain. The TA domain is necessary for viral DNA replication, interaction with the viral E1 protein and mediating transcriptional activation. In addition, the TA domain is required for the association of E2 with mitotic chromosomes to ensure the maintenance of the viral DNA in dividing cells (4, 21, 30, 34, 42). Specific mutations in the TA domain have been shown to disrupt the tethering of viral genomes to mitotic chromosomes (1, 5, 51).

We have recently shown that Brd4 (bromodomain containing protein 4) mediates the association of BPV1 E2 to mitotic chromosomes and that the binding of E2 to Brd4 is conserved among the papillomaviruses (48). Through an interaction of the carboxy-terminal region of Brd4 with the amino-terminal TA domain of E2, this protein complex serves to bridge the viral DNA with cellular mitotic chromosomes (5, 7, 32, 48). Brd4 is a member of the BET family, a group of structurally related proteins characterized by the presence of two bromodomains and one extra-terminal (ET) domain of unknown function. Bromodomains in general have been shown to interact with acetylated lysines in histones and are involved in chromatin targeting and remodeling (12, 23, 50). Unlike other bromodomain proteins that are released from chromatin during mitosis, BET family members remain bound to chromatin during mitosis (13, 25). Mouse embryos nullizygous for Brd4 die shortly after implantation, suggesting a role for Brd4 in fundamental cellular processes (19). Recently Brd4 has been shown to influence the general RNA polymerase II dependent transcription machinery by interacting with the core factors of the positive transcription elongation factor b (P-TEFb) and the Mediator complex (22, 46). In addition, Brd4 binds to acetylated chromatin with preferential binding for acetylated histones H3 and H4 (12). The mechanism regulating the recruitment of Brd4 to promoters however is not yet well understood.

In order to gain insight into the functions of the Brd4/E2 complex, we analyzed a series of E2 single amino acid alanine scanning mutants for their abilities to bind Brd4. These experiments permitted us to map the face of the E2 TA domain involved in binding Brd4 which turned out to be distinct from the region of the E2 TA required for E1 binding and for enhancing E1-dependent viral DNA replication (2, 9, 10, 14, 15, 40). Instead, the amino acids involved in Brd4 binding corresponded well with those necessary for the E2 transcriptional activation function. In combination with functional studies utilizing the C-terminal domain of Brd4 as dominant negative inhibitor of the E2/Brd4 interaction and with siRNA knock down experiments of Brd4 we have demonstrated that Brd4 is required to mediate its transcriptional activation function.

Analysis of Brd4 Binding to a Series of HPV16 E2 Mutants

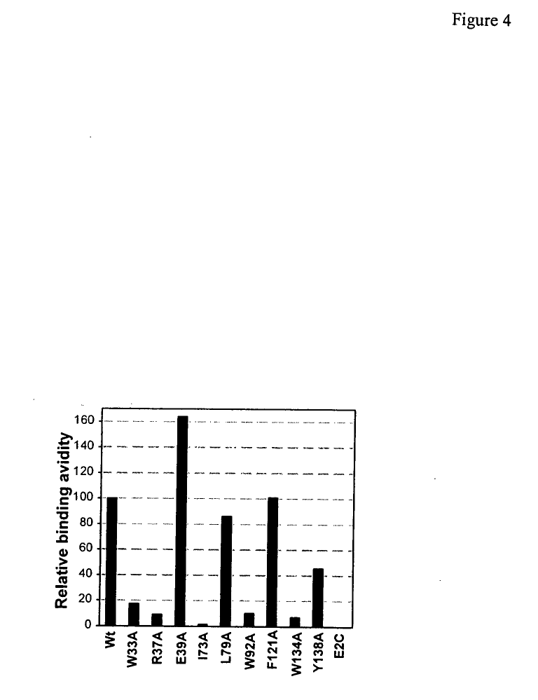

The N-terminal transactivation domain of E2 mediates the binding to Brd4 (48). To further characterize this interaction we assayed single amino acid alanine substitution mutants within the HPV16 E2 transactivation domain for their abilities to bind Brd4. Specific amino acid mutants within the domain were selected for analysis based on the structure of this region (3, 17) and a previous analysis of amino acids conserved within this domain among different papillomaviruses (40). Our goal was to map the amino acids on the surface of the E2 transactivation domain required for Brd4 binding and to determine whether Brd4 binding correlated with any other E2 functions. In order to assess Brd4 binding, GST pull down and coimmunoprecipitation assays were performed with individual E2 mutants (FIG. 4). For the GST pull down assays GST-tagged wild type and mutant E2 proteins were expressed and purified from E. coli. Each protein was incubated with an aliquot of in vitro translated, ³⁵S-labeled Brd4-CTD and bound material was separated using glutathione-sepharose. The wildtype E2 served as a positive control and the C-terminal 171 aa of E2 (E2C) and GST alone (GST) served as negative controls. Bound proteins were separated on SDS-page gels and radioactive proteins were visualized and quantified by a phosphoimager (FIG. 4). In parallel, equal amounts of the GST-E2 proteins were run on separate gels as input controls and visualized by Coomassie blue staining. In these experiments the carboxy terminus of E2 (E2C), spanning amino acid 153 to 365, served as a negative control since it lacks the TA domain.

In the experiment, E2(wt) bound 28% of the input Brd4-CTD. We found that the E39A, L79A and F121A mutants bound Brd4 comparably to wt E2. In contrast, 173A did not bind Brd4 and the W33A, R37A, W92A and W134A mutants bound less than 20% of Brd4 compared to wild type E2. In these experiments, intermediate binding of Brd4 was observed for the Y138A mutant.

We next examined the capacity of the E2 mutants to bind Brd4 in vivo by examining co-immunoprecipitations of the E2 mutants and Xpress-tagged Brd4-CTD that were co-expressed in C33A cells. Protein complexes were isolated using an antibody directed to the C-terminus of E2, and binding was quantitated by an Odyssey Infrared Imaging system using an anti-Xpress antibody (Leicor). In agreement with the GST pull down experiments, 173A did not bind Brd4 and the W33A, R37A and W92A mutants were significantly impaired (<10% of wild type E2) in their ability to bind the Brd4-CTD. The E39A, K68A, L79A, E90A, T93A, F121A, D122A, Y138A and Y178A mutants efficiently bound the Brd4-CTD at levels greater than 40% of wt E2. A summary of the Brd4 binding results from both experiments is shown in Table 2. These binding studies revealed a nearly complete correlation between amino acids important for transcriptional activation and Brd4 binding. In contrast, no correlation was seen between Brd4 binding and E1 binding or DNA replication. For example, the E2 mutant 173A does not bind Brd4 and is inactive in transcriptional activation, but has wild type activities for E1 binding and viral DNA replication. On the other hand, the E2 mutant E39A has a high affinity for Brd4 and can activate transcription, but is defective for E1 binding and viral DNA replication.

TABLE 2

| | | Brd4 | DNA | E1 |
|---|---|---|---|---|
| | transactivation | binding | replication | binding |
| wt | +++ | +++ | +++ | +++ |
| W33A | + | + | + | + |
| R37A | − | − | + | +++ |
| E39A | +++ | ++/+++ | − | − |
| K68A | ++ | ++ | ++ | +++ |
| I73A | − | − | +++ | +++ |
| L79A | +++ | +++ | +++ | ++ |
| E90A | +++ | +++ | +++ | +++ |
| W92A | − | − | + | + |
| T93A | +++ | ++ | ++ | +++ |
| F121A | +++ | +++ | + | + |
| D122A | +++ | +++ | ++ | + |
| W134A | − | − | + | + |
| Y138A | ++ | +++ | + | ++ |
| Y178A | +++ | ++/+++ | + | + |

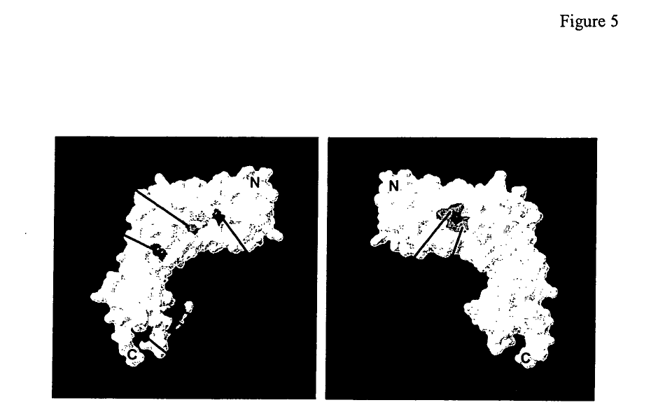

FIG. 5 shows a structural model of the HPV16 E2 transactivation domain (3) in which the amino acids R37 and 173 important for Brd4 binding and transactivation have been colored in red. Residues E39, F121, D122 and Y178 which are required for E1 binding and viral DNA replication are indicated in blue. W33A and W134A (shown in purple) are significantly impaired in each E2 function tested and therefore it is possible that mutation of these residues might affect the overall structure of E2. The structural model shows clearly that amino acids required for Brd4 binding and for transcriptional activation cluster on one side of the E2 surface, whereas amino acids necessary for E1 binding and viral DNA replication map to a different side of the E2 protein. These data suggested that Brd4 could be involved in the transcriptional activation function of E2, and that conversely it was unlikely to be important for its viral DNA replication function.

Brd4-CTD does not Influence the Growth Properties of C33A Cells

In order to test the potential role of Brd4 binding on the E2 transcriptional activation and viral DNA replication functions, we performed E2 dependent transcriptional reporter assays and transient DNA replication assays in presence or absence of the Brd4-CTD, a dominant acting negative inhibitor of Brd4/E2 binding. We first asked whether expression of the Brd4-CTD influenced cellular proliferation or cellular DNA replication of C33A cells by examining its effect on cellular growth rates, by an Alamar Blue assay, and by BrdU incorporation.

C33A cells were co-transfected with a plasmid expressing Brd4-CTD or an empty vector and a puromycin resistance plasmid. Cells were split and placed under puromycin selection. Cells from triplicate plates were counted each day with a hemocytometer.

No difference in the growth rates of C33A cells transfected with empty vector or transfected with a Brd4-CTD expression vector was observed. To determine whether expression of the Brd4-CTD affected cellular metabolic activity, C33A cells transfected with Brd4-CTD was compared to C33A cells transfected with an empty vector using an alamarBlue reduction assay. Equal numbers of cells were incubated with 10% alamarBlue reagent. Fluorescence emission (FE) was measured after 0, 2, 4, 6, 8, 20, 22, 26 and 32 h and plotted against the incubation time.

Like MTT [3-(4,5-dimethyldiazol-2-yl)-2,5 diphenyl Tetrazolium Bromid], AlamarBlue is reduced by metabolic intermediates such as NADPH, FADH and NADH, and changes from an oxidized non-fluorescing to a reduced fluorescing state. We found no evidence that Brd4-CTD expression affected the metabolic state of C33A cells. Since a change in cellular DNA replication could potentially mask changes measured in a viral DNA replication assay, we performed a BrdU incorporation assay to examine the affect of Brd4-CTD on cellular DNA replication.

BrdU incorporation was assayed in 500, 2×10³, 6×10³ and 14×10³ C33A cells stably expressing the Brd4-CTD or vector alone. Cells were labeled for 2 h with BrdU, and incorporation was detected with a peroxidase conjugated anti-BrdU antibody. Chemiluminescence was measured with a luminometer. The relative light units/second (rlu/s) were plotted in correlation to the number of cells plated.

We found no difference in C33A cells expressing the Brd4-CTD compared to the vector control cell line. Furthermore, it should be noted that the Brd4-CTD has no effect on E2 levels of expression or localization (48).

The E2 Viral DNA Replication Function is Independent of Brd4

Some of the E2 mutants, like E39A, F121A, Y138A and Y178A bound Brd4 well, but are incapable of supporting viral DNA replication, suggesting that viral DNA replication is not dependent upon the ability of E2 to bind Brd4. In order to test the role of Brd4 binding on the E2 viral DNA replication function directly, we analyzed the effect of Brd4-CTD in viral DNA replication assays. The viral DNA replication activity was measured after cotransfection of a papillomavirus origin containing plasmid (p16ori), E1 and E2 expression plasmids and either a Brd4-CTD expression plasmid or an empty vector.

Transient in vivo replication assay of a HPV16 origin containing plasmid (p16ori) was conducted as follows. C33A cells were transfected with p16ori, along with plasmids expressing E1 and/or E2 and a plasmid expressing the Brd4-CTD or vector control. Each assay was done separately in triplicate. Low molecular weight DNA was harvested by the Hirt method 48 h following transfection. DNA was digested with DpnI and DNA was analyzed by Southern blot hybridization using a HPV16 ori probe. As negative controls, replication assays without E1 or E2 or without the origin containing plasmid were performed.

We observed no significant inhibition of the E2 enhanced p16ori dependent plasmid replication by the Brd4-CTD. In addition, transfection of a full length Brd4 expression plasmid had no effect on E2 enhanced p16ori dependent plasmid replication (data not shown). Furthermore, we found that the Brd4-CTD did not compete E1 binding to E2 in vitro (data not shown). We therefore conclude that E2 binding to Brd4 is not required for its DNA replication function.

Brd4-CTD Inhibits the E2 Transcriptional Activation Function

The mutational analysis of the N-terminal domain of E2 described earlier indicated that Brd4 binding correlated with transcriptional activation functions of the E2 protein. To examine whether Brd4 indeed plays a fundamental role in E2 dependent transcriptional activation, we next tested whether the Brd4-CTD could affect E2 dependent transcriptional activation. C33A cells were transfected with an E2 responsive reporter plasmid (p2×2xE2BS-Luc), which contains four E2 binding sites, the E2 expression plasmid and increasing amounts of a Brd4-CTD expression plasmid.

C33A cells were transfected with p2×2xE2BS-Luc, an E2 dependent luciferase reporter plasmid. Coexpression of the E2 wt activates the reporter plasmid. Luciferase activity was also measured in presence of increasing amounts of Brd4-CTD (0.0014 μg to 1.4 μg in 10 fold increments). The luciferase activities were normalized for transfection efficiency determined by the β-galactosidase activity expressed in the cotransfected cells.

By itself, E2 enhanced the luciferase expression from the reporter plasmid 60-fold. Expression of Brd4-CTD inhibited the transcriptional activation function of E2 in a dose-dependent manner. Brd4-CTD alone had no significant effect on the expression of luciferase from the reporter plasmid in the absence of E2.

To address the specificity of the inhibition of E2 transactivation by the Brd4-CTD, two additional reporter plasmids that are not responsive to E2 were tested: an interferon β (IFNβ)-promoter luciferase reporter plasmid that responds to the interferon regulatory factor 3 (IRF3) and a PIN1-promoter luciferase reporter plasmid that responds to the transcription factor E2F.

C33A cells were transfected with E2 (p2×2xE2BS), IRF-3 (IFNβ) or E2F (PIN1) dependent luciferase reporter plasmids. Luciferase activity was stimulated by cotransfection of the corresponding activator plasmid E2, IRF-3 or E2F, respectively. In addition each assay was performed by cotransfection of the Brd4-CTD (1.4 μg) expressing plasmid. The luciferase activities were normalized for transfection efficiency by the β-galactosidase activity expressed in the cotransfected cells.

In contrast to the strong inhibition of E2 transcriptional activation by Brd4-CTD, no inhibition by the Brd4-CTD was observed for either IRF3 activation of the IFNβ promoter or PIN1 promoter.

Brd4 is Required for E2 Transcriptional Activation

To determine whether the inhibition of E2 transcriptional activation by the Brd4-CTD was due to competition of the full length Brd4 protein or to some other cellular factor that bound to the same region of the E2 TA domain, we tested whether the full length Brd4 protein (Brd4-FL) could rescue this inhibition.

C33A cells were transfected with an E2-dependent luciferase reporter and the E2 expression plasmid. Cells were cotransfected with 0.7 μg Brd4-CTD and 0.7 or 2.3 μg of full-length Brd4, respectively. The luciferase activities were normalized for transfection efficiency as determined by the β-galactosidase activity expressed in the cotransfected cells.

The full-length protein was able to rescue the inhibition by Brd4-CTD in a dose dependent manner. Therefore we conclude that Brd4 has an essential role in the E2 transcriptional activation function.

To further examine the requirement for Brd4 in E2 transcriptional activation, we employed short interfering RNAs (siRNAs) to knock down endogenous Brd4 expression in C33A cells. Constructs that generated siRNAs directed against the N-terminus [siRNA-Brd4(NT)] or the C-terminus [siRNA-Brd4(CT)] of Brd4 were constructed using pSUPER vectors. These plasmids were then tested for their abilities to knock down Brd4 protein levels by immunofluorescence. C33A cells were transfected with either the empty vector or one of the siRNA expressing plasmids along with an enhanced green fluorescent protein (EGFP) expression plasmid at a ratio of 15:1. The cells were stained with an anti-Brd4 antibody and counterstained with DAPI. The siRNA-Brd4(NT) and siRNA-Brd4(CT) expression plasmids each significantly decreased the Brd4-specific signal in the transfected cells. In contrast, cells transfected with the empty vector did not show any difference in Brd4 levels compared to nontransfected cells. We next tested whether knockdown of Brd4 using the siRNA-Brd4(NT) and siRNA-Brd4(CT) expression plasmids could inhibit E2 transcriptional activation. The Brd4 siRNA expression plasmids, as well as a GFP siRNA expression plasmid as the negative control, were co-transfected with the E2 dependent luciferase reporter plasmid (p2×2xE2BS). Each of the Brd4 siRNAs constructs, either alone or in combination, strongly inhibited E2 dependent transcriptional activation around 85% compared to the GFP siRNA control. Since two independent siRNA constructs for Brd4 strongly inhibited E2 transcriptional activation, we conclude that the result is unlikely a consequence of off-target effects of the siRNA constructs. Furthermore to test specificity, we examined the effects of these two siRNAs on the IRF3 activation of the IFNβ1-luciferase reporter and found minimal effects. Taken together, these results show that Brd4 is required for papillomavirus E2 transcriptional activation and specifically mediates its transcriptional activation function.

Discussion

The papillomavirus (PV) E2 proteins have well characterized regulatory functions affecting viral transcription, viral DNA replication and long-term plasmid maintenance. Our laboratory has identified Brd4 as the cellular mitotic chromosome associated factor that mediates the chromosome binding of E2 (48). In addition, recent studies have shown that stable PV based plasmid maintenance by E2 in yeast requires Brd4 and that Brd4 binding to E2 is necessary for the mitotic chromosome localization of E2 (5, 7). Furthermore, we have recently shown that blocking the interaction of E2 with Brd4 enhances viral genome loss and enhances the phenotypic reversion of bovine papillomavirus transformed cells (49).

In this study we have found that Brd4 is required for the transcriptional activation function of E2. This was first suggested by our protein interaction studies using a functionally well-characterized series of alanine scanning point mutants of the N-terminal TA domain of HPV16 E2 (Sakai et al., 1996). Mutants defective for their ability to transactivate an E2-responsive promoter were impaired in binding to Brd4 (Table 2). Visualization of amino acids necessary for Brd4 binding on the structural model of the HPV16 E2 transactivation domain revealed that these amino acids are localized on a different face of E2 than that involved in binding E1 (FIG. 5). As expected, E1/E2 binding was not competed by the Brd4-CTD and the Brd4-CTD did not significantly affect viral DNA replication.

Two recent studies had used a mutational analysis of the E2 TA domain to identify E2 mutants defective for localization to mitotic chromosomes. Baxter and colleagues used mutant BPV-1 E2 proteins to study the mitotic chromosome binding activity and binding to Brd4 (5). In their study they used a combination of multiple site point mutations as well as a series of single amino acid substitution mutants (R37A, E39A, R68A and 173A). Consistent with our results they found that E39A and R68A bound mitotic chromosomes at wild type levels whereas 173A was nearly completely excluded from mitotic chromosome (5). Abroi et al also examined a series of BPV-1 E2 TA domain mutants for a variety of functions (1). Their study predated our publication of the E2/Brd4 interaction, so it did not include an analysis of Brd4 binding. The results published by Abroi et al differed from those of Baxter et al. The discrepancy between the findings of these 2 groups could perhaps be explained by the different experimental conditions used in each study. As reported by Zheng et al, lowering the temperature or using agents that promote protein folding may increase the ability of some mutant E2 proteins to associate with mitotic chromosomes (51). In our studies we were able to avoid these difficulties from previous reports by using a dominant negative inhibitor of the Brd4/E2 interaction, the Brd4-CTD, and by siRNA knock down experiments.

Based on the correlation we observed between Brd4 binding and the transcriptional activation capacity of the E2 TA mutants, we tested whether the Brd4-CTD could inhibit E2 transactivation of an E2 responsive promoter. We found a dose dependent inhibition of the E2 transactivation activity by the Brd4-CTD with nearly complete inhibition at the highest concentration. This inhibition was specific for E2 transactivation and was rescued by co-expression of the full-length Brd4 protein. The importance of Brd4 in the E2 transcriptional activation function was further validated by the Brd4 knockdown experiments employing siRNAs to Brd4.

E2 is an essential regulatory factor for the papillomaviruses. Of the various E2 functions, its transcriptional activities are perhaps the least well understood at a mechanistic level. E2 can either activate or repress a promoter containing E2 binding sites depending upon the number and position of the binding sites within the promoter region (20). The E2 protein has been shown to bind a number of general cellular transcription factors such as TFIIB and TBP, transcriptional coactivators AMF-1 (activation domain modulating factor 1), p/CAF and p300/CBP and the nucleosome assembly protein NAP-1 (8, 28, 29, 35, 36, 47). It has also been shown that a direct interaction between the transcription factor Spl and E2 brings distantly bound E2 to the promoter region through formation of stable DNA loops (31). Interestingly, even though E2 interacts with a large number of cellular proteins over its E2 TA domain, until now no complete correlation between the transcriptional activation domain and the binding of another protein on the E2 TA domain has been demonstrated as there is for Brd4. The association sites of E2 TA with cellular transcription cofactor AMF-1 (amino acids 134-216) and TFIIB (amino acids 74-134) for example are distinct from the transcriptional activation domain, indicating Brd4 as mediator of the E2 dependent transcriptional activation (8, 35, 47). Brd4 on the other side has recently been shown to be a component of the positive transcription elongation factor b (P-TEFb) complex and to interact with subunits of the Mediator complex (19, 22, 24, 46). It seems therefore reasonable to surmise that Brd4 may link the transcription factor E2 with the P-TEFb and Mediator complexes, thus connecting E2 to the general transcription machinery. It is possible that Brd4 regulates the recruitment of the transcription machinery to specific genes through interactions with certain transcription factors such as E2 but not with others.

In summary, this study has identified Brd4 as the mediator of E2 dependent transcriptional activation. Functional disruption of the Brd4/E2 interaction by a dominant negative inhibitor specifically abolished E2 dependent transcriptional activation whereas other E2 dependent functions, like viral DNA replications remain unaffected. Furthermore depletion of Brd4 by knock down experiments validated the role of Brd4 for E2's transactivation function. Given the importance of the transcriptional activation function of E2 to the papillomavirus life cycle, this study further highlights the binding of E2 to Brd4 as a potential target for the development of specific papillomavirus inhibitors.

Materials and Methods

Recombinant Plasmids.

The eukaryotic pCMV4-16E2 expression vectors for wild type (p3662) and mutant E2 proteins (p3665 to p3688) and and pCMV-16E1 (p3692) proteins have been described earlier (40). The E. coli pGEX-2T-16E2 expression plasmids (p3798 to p3809) were derived from the wild type pGEX-2T-16E2 (p3796) plasmid (40). Plasmids containing the full length human Brd4 (pcDNA4C-Brd4-FL) or the C-terminal domain between aa 1047 and 1362 (pcDNA4C-SV40NLS-hBrd4-CTD) and p2×2xE2BS-Luc and p16ori have been described previously (26, 40, 48). The IFNβ promoter luciferase, the PIN1 promoter luciferase reporter, the IRF3 expression plasmid and the E2F expression plasmid have been described earlier by J. Hiscott and L. V. Ronco, respectively (38, 39, 41). pSVβ-GAL and pEGFP were purchased from BD Bioscience.

Antibodies

Rabbit polyclonal Brd4 antibody was raised against an N-terminal Brd4 fragment (amino acid 1 to 470) followed by affinity purification. The HPV16 E2C antibody has been described previously (40). The anti-XPress-tag antibody was purchased from Invitrogen.

Cell Lines and Transfections

The human cervical cancer cell lines HeLa (HPV18 positive) and C33A (HPV negative) were maintained in Dulbecco's modified Eagle's medium (Invitrogen) with 10% fetal calf serum (Hyclone). Cells were tested to be mycoplasma negative (Mycoplasma PCR Elisas, Roche). Plasmid DNAs were introduced into cells by Fugene 6 transfection reagent (Roche). Using standard retrovirus production and transfection procedures, pLPCX-HXNCTD or the empty vector pLPCX were used to generate C33A cells stably expressing Brd4-CTD or the empty vector, respectively.

Cellular Growth Proliferation Assay

Approximately 2×10⁶ C33A cells were co-transfected with combinations of 0.6 μg pBABE-puro, 0.4 μg pEGFP, 2 μg pCMV-E2 and 1.5 μg pcDNA4C-Brd4-CTD. 24 hrs after transfection, cells were split to 3×10⁴ cells per 35 mm dish and put under puromycin-selection (0.3 μg/μl). Cells were trypsinized and counted daily in triplicate.

AlamarBlue Assay

Approximately 6×10³ C33A cells stably transfected with the Brd4-CTD were incubated with DMEM containing 10% almarBlue (Biosource) solution for 0, 2, 4, 6, 8, 20, 22, 26 and 32 h. Fluorescence measurements were performed with a Cytofluor Multiwell Plate Reader (PerSeptive Biosystems) by exciting at 530 nm and measuring emission at 590 nm.

The fluorescence emission intensity units were plotted as a function of incubation time.

BrdU Incorporation Assay

The BrdU incorporation assay was performed as recommended by the manufacturer (BrdU Cell proliferation ELISA, Roche). In brief, stably transfected C33A cells were split to 500, 2×10³, 6×10³ and 14×10³ cells per 96-well. Cells were incubated for 2 hours with BrdU, followed by fixation for 30 min and an incubation for 1 h with anti-BrdU peroxidase conjugated antibody. After addition of luminol (substrate) chemiluminescence was measured with a luminometer and expressed as a function of cell number.

RNA Interference

For knock down of Brd4 expression, the pSUPER vector system was used (OligoEngine). Following the manufacturer's protocol, siRNA expression plasmids were constructed by annealing oligos containing the siRNA-expressing sequence, for siRNA-Brd4(NT) 5′-GACACTATGGAAACACCAG-3′, for siRNA-Brd4(CT) 5′-GCGGGAGCAGGAGCGAAGA-3′, and cloning them in the BglII/HindIII sites of the vector. The control siRNA-GFP construct was a gift from B. Lilley and targeted the sequence 5′-GCAAGCTGACCCTGAAGTTC-3′(45). SiRNA-induced silencing was determined by indirect immunofluorescence of Brd4. Cells were cotransfected with 2 μg siRNA and 0.13 μg EGFP expression plasmids. After 36 hours cells were plated on cover slips and 24 h later fixed with 3% paraformaldehyde. Staining of Brd4 with anti-Brd4 antibody was performed as described earlier (48). As secondary antibody a Alexa Fluor 594 goat anti-rabbit antibody (Molecular Probes) was used. Cells were counterstained with DAPI and examined with a Leica DMLB epifluorescence microscope.

GST Pulldown

Glutathione S-transferase (GST) E2 fusion proteins were expressed in E. coli BL21. Proteins were affinity purified with glutathione sepharose 4B beads (Amersham) per manufacturer recommendations. Proteins were eluted from the columns with 10 mM glutathione and dialyzed over night in 150 mM NaCl, 50 mM Tris-HCl pH8.0, 1 mM DTT. The purity of the GST fusion proteins was confirmed by SDS gelelectrophoresis and Coomassie staining.

³⁵S-labeled Brd4-CTD was generated by using the T7-TNT coupled rabbit reticulocyte lysate (RRL) system (Promega). Briefly, GST pulldowns were performed as follows: 0.5 μg of each GST-E2 fusion protein and 15 μl of the in vitro translated Brd4-CTD were incubated in 500 μl binding buffer (20 mM Tris-HCl pH7.5, 50 mM NaCl, 4 mM MgCl₂, 2 mM DTT, 0.5% NP-40, 2% nonfat dry milk) for 60 min at 4° C. 20 μl of 50% GST sepharose slurry equilibrated in binding buffer was added and incubation was continued for 30 min. Beads were sedimented and washed three times with 1 ml of binding buffer without dry milk. The samples were analyzed by SDS-polyacrylamide gel electrophoresis and the labeled proteins were visualized by autoradiography.

Coimmunoprecipitation and Western Blot Analysis

For coimmunoprecipitation, 1×10⁷ transiently transfected C33A cells were harvested 48 to 72 hrs after transfection, and soluble proteins were extracted as described previously (Schreiber 1989). The extract (1.5 ml) was mixed with 30 μl 50% protein A agarose slurry (Invitrogen) in IP-binding buffer (10 mM HEPES pH7.9, 10 mM KCl, 50 mM NaCl, 0.1 mM EDTA, 0.1 mM EGTA, 1 mM DTT) and precleared overnight at 4° C. Subsequently the extracts were incubated with 4 μl of affinity-purified anti-E2C-antibody (40) for 4 hrs at 4° C., followed by the precipitaiton of the antigen-antibody compexes with 30 μl 50% protein A agarose slurry (Invitrogen). The beads were washed three times with IP-binding buffer and bound proteins were eluted with 30 μl sample buffer. Aliquots were resolved on a 8% SDS-polyacrylamide gel. Proteins were transferred to a PVDF membrane and blotted with anti-Xpress mouse monoclonal antibody (Invitrogen) and anti-HPV16E2 rabbit polyclonal antibody (Sakai 1996). As secondary antibodies, fluorescent (680 nm and 750 nm) labeled anti-mouse and anti-rabbit antibodies were used (Molecular Probes). Western blots were visualized and quantitated with an Odyssey Infrared Imaging system (Leicor).

Transient Papillomavirus DNA Replication Assay

A transient papillomavirus DNA replication assay was performed following a protocol described by Del Vecchio et al. (11). C33A cells were co-transfected with the papillomavirus origin containing plasmid (p16ori) and plasmids expressing E1 and E2 (pCMV-16E1, pCMV-16E2). Around 48 hours after the transfection, low molecular weight DNA was prepared using the Hirt method followed by phenol-chloroform extraction and ethanol precipitation (18). Since DNA replicated in eukaryotic cells is not methylated on adenine residues and is resistant to DpnI digestion, the replicated DNA is distinguished from input DNA by a DpnI digestion. The digested samples were then analyzed by Southern blotting using a probe encompassing the PV origin of replication labeled with ³²P using a random prime labeling kit (Stratagene). The blot was washed twice with a low stringency buffer (2×SSC, 0.1% SDS) for 1 h at room temperature and once with a high stringency buffer (0.1×SSC, 0.1% SDS) for 1 h at 63°. Blots were dried and visualized by autoradiography. Quantification was performed using a Storm PhosphoImager (Molecular Dynamics).

Reporter Assays

Approximately 2×10⁶ C33A cells were transfected with 0.7 μg p2×2×E2BS-Luc and 1.4 μg pCMV-E2. To determine or normalize transfection efficiencies, 0.5 μg pSVβ-GAL and 0.4 μg pEGFP were co-transfected. To test the influence of Brd4-CTD on E2 transcriptional activation, cells were co-transfected with 0.0014 μg, 0.014 μg, 0.14 μg and 1.4 μg of pcDNA4C-Brd4-CTD. For rescue experiments with the full-length Brd4 protein 0.7 μg pcDNA4C-Brd4-CTD and 0.7 μg or 2.3 μg pcDNA4C-Brd4-FL were used. 48 hrs after transfection, cells were lysed and luciferase activities were measured according to the manufacturer's protocol (Luciferase assay system, Promega). Luciferase activities were normalized to the β-galactosidase activity of pSVβ-GAL cotransfected with the reporter plasmid (Luminescent β-Gal Detection Kit II, BD Bioscience).

### REFERENCES

1. Abroi, et al. (2004) J Virol 78:2100-2113. 2. Abroi. et al. (1996) J Virol 70:6169-6179. 3. Antson, et al. (2000) Nature 403:805-809. 4. Bastien, et al. (2000) Virology 270:124-134. 5. Baxter, et al. (2005) J Virol 79:4806-4818. 6. Berg, M. et al (1997) J Virol 71:3853-3863. 7. Brannon, et al. (2005) Proc Natl Acad Sci USA 102:2998-3003. 8. Breiding, et al. (1997) Mol Cell Biol 17:7208-7219. 9. Brokaw, et al. (1996) J Virol 70:23-29. 10. Cooper, et al. (1998) Virology 241:312-322. 11. Del Vecchio, et al. (1992) J Virol 66:5949-5958. 12. Dey, et al. (2003) Proc Natl Acad Sci USA 100:8758-8763. 13. Dey, A. et al. (2000) Mol Cell Biol 20:6537-6549. 14. Ferguson, et al. (1996) J Virol 70:4193-4199. 15. Grossel, et al. (1996) J Virol 70:7264-7269. 16. Guex, et al. (1997) Electrophoresis 18:2714-2723. 17. Harris, et al. (1999) Science 284:1673-1677. 18. Hirt, et al. (1967) J Mol Biol 26:365-369. 19. Houzelstein, et al. (2002) Mol Cell Biol 2 2:3794-802. 2 0. Howley, et al. (2001) Fields Virology, 4th ed. Lippincott Williams & Wilkins, Philadelphia. 21. Ilves, et al. (1999) J. Virol 73:4404-4412. 22. Jang, et al. (2005) Mol Cell 1 9:523-534. 2 3. Jeanmougin, et al. (1997) Trends Biochem Sci 22:151-153. 24. Jiang, et al. (1998) Proc Natl Acad Sci USA 95:8538-8543. 25. Kanno, et al. (2004) Mol Cell 13:33-43. 26. Kovelman, et al. (1996) J Virol 70:7549-7560. 27. Law, et al. (1981) Proc Natl Acad Sci USA 78:2727-2731. 28. Lee, et al. (2002) J. Biol Chem 277:6483-6489. 29. Lee, et al. (2000) J Biol Chem 275:7045-7051. 30. Lehman, et al. (1998) Proc Natl Acad Sci USA 95:4338-4343. 31. Li, et al, (1991) Cell 65:493-505. 32. McPhillips, et al. (2005) J Virol 79:8920-8932. 33. Mohr, et al. (1990) Science 250:1694-1699. 34. Piirsoo, et al. (1996) EMBO J. 15:1-11. 35. Rank, et al. (1995) J Virol 69:6323-6334. 36. Rehtanz, et al. (2004) Mol Cell Biol 24:2153-2168. 37. Romanczuk, et al. (1990) J Virol 64:2849-2859. 38. Ronco, et al. (1998) Genes Dev 12:2061-2072. 39. Ryo, et al. (2002) Mol Cell Biol 22:5281-5295. 40. Sakai, et al. (1996) J Virol 70:1602-1611. 41. Servant, et al. (2001) J Biol Chem 276:355-363. 42. Skiadopoulos, et al. (1998) J Virol 72:2079-2088. 43. Spalholz, et al. (1985) Cell 42:183-191. 44. Thierry, et al. (1987) Embo J 6:3391-3397. 45. Tiscornia, et al. (2003) Proc Natl Acad Sci USA 100:1844-1848. 46. Yang, et al. (2005) Mol Cell 19:535-545. 47. Yao, et al. (1998) J Virol 72:1013-1019. 48. You, et al. (2004) Cell 117:349-360. 49. You, et al. (2005) J. Virol. 79: in press. 50. Zeng, et al. (2002) Lett 513:124-128. 51. Zheng, et al. (2005) J Virol 79:1500-1509. 52. zur Hausen, et al. (2001) Oncogene 20:7820-7823.
   Equivalents

The present disclosure provides among other things methods and compositions for the prevention and/or treatment of viral infections. While specific embodiments have been discussed, the above specification is illustrative and not restrictive. Many variations of the methods, compositions, and process disclosed herein will become apparent to those skilled in the art upon review of this specification. The appended claims are not intended to claim all such embodiments and variations, and the full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: S C Verma & E S Robertson, FEMS Microbiol Lett 222: 155-63 (2003); N Bastien & A A McBride, Virology 270: 124-134 (2000); M H Skiadopoulos & A A McBride, J. Virology 72: 2079-2088 (1998); C French et al., Am. J. Pathology 159: 1987-1992 (2001); D Houzelstein et al., Mol. Cell. Biol. 22: 3794-3802 (2002); T Maruyama et al., Mol. Cell. Biol. 22: 6509-6520 (2002); A Dey et al., Mol. Cell. Biol. 20: 6537-6549 (2000); Sakai et al., J. Virology 70: 1602-1611 (1996); J. Choe, et al., J. Virology 63: 1743-1755 (1989); D. Francis, et al., J. Virology, 74: 2679-2686 (2000); N. Frank et al., J. Virology 69: 6323-6334 (1995); S. Vande Pol et al., J. Virology 66: 2346-2358 (1992); T. Zemlo et al., J. Virology 68: 6787-6793 (1994); Accession No. gi60965 (X02346); Accession No. gi3041739 (P03122); U.S. Pat. Nos. 6,573,364, 6,420,118, and 6,399,075; and U.S. Patent Application Publication Nos. 2003/0027768 A1, 2003/0103997 A1, and 2002/0099022 A1.

1. A polypeptide consisting essentially of an amino acid sequence that is at least about 90% identical to amino acids 1047-1362, 1134-1362 or 1224-1362 of SEQ ID NO: 2, wherein the polypeptide binds to E2 or to a latency-associated nuclear antigen (LANA) protein from a Kaposi sarcoma-associated herpesvirus (KSHV).

2. The polypeptide of claim 1, linked to a heterologous peptide.

3. The polypeptide of claim 2, wherein the heterologous peptide is selected from the group consisting of His, myc, HA, GST, protein A, protein G, calmodulin-binding peptide, thioredoxin, maltose-binding protein, poly arginine, poly His-Asp, FLAG, or a portion of an immunoglobulin protein, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine, or red fluorescent protein from discosoma (dsRED).

4. An isolated nucleic acid that encodes the polypeptide of claim 1, but which does not encode the full length protein having SEQ ID NO: 2.

5. The nucleic acid of claim 4, operably linked to a transcriptional regulatory sequence.

6. A vector comprising the nucleic acid of claim 5.

7. A host cell comprising the vector of claim 6.

8. An antibody that binds to the polypeptide of claim 1.

9. A complex or composition comprising a polypeptide comprising an amino acid sequence that is at least about 90% identical to SEQ ID NO: 2 or 4 or a portion thereof sufficient for binding to an E2 protein or to a LANA protein; and an E2 protein or LANA protein or a portion thereof sufficient for binding to a polypeptide comprising SEQ ID NO: 2 or 4.

10. The complex or composition of claim 9, wherein the E2 protein is from a papilloma or herpes virus.

11. A method for identifying an agent that disrupt the binding of a Brd4 protein to an E2 protein or LANA protein, comprising (i) contacting a protein comprising an amino acid sequence that is at least about 90% identical to SEQ ID NO: 2 or 4 or a portion thereof sufficient for binding to an E2 protein and an E2 or LANA protein or a portion thereof that is sufficient for binding to a Brd4 protein in the presence of a test agent; and (ii) determining the level of interaction between the two proteins or portion thereof, wherein a lower level of interaction in the presence of the test agent relative to its absence indicates that the test agent is an agent that disrupts the interaction between a Brd4 protein and an E2 protein.

12. The method of claim 11, wherein the protein comprising an amino acid sequence that is at least about 90% identical to SEQ ID NO: 2 or 4 or a portion thereof sufficient for binding to an E2 protein is a protein consisting essentially of an amino acid sequence that is at least about 90% identical to amino acids 1047-1362, 1134-1362 or 1224-1362 of SEQ ID NO: 2.

13. A method for treating a subject suffering from a viral related disease or disorder, comprising administering to subject in need thereof a therapeutically effective amount of a polypeptide of claim 1 or a nucleic acid encoding such or an antibody that inhibits the interaction between a Brd4 protein and an E2 protein.

14. The method of claim 13, wherein the subject has a disease or disorder related to an infection of a papillomavirus or a herpes virus.
